(19) United States
(12) Patent Application Publication
(10) Pub. No.: US 2020/0069236 A1
(43) Pub. Date: Mar. 5, 2020

(54) METHODS AND SYSTEMS FOR DIAGNOSIS OF POST-TRAUMATIC STRESS DISORDER

(71) Applicant: THE UNITED STATES OF AMERICA AS REPRESENTED BY THE DEPARTMENT OF VETERANS AFFAIRS, Washington, DC (US)

(72) Inventor: Mo Modarres, Tampa, FL (US)

(21) Appl. No.: 16/467,666

(22) PCT Filed: Dec. 11, 2017
(86) PCT No.: PCT/US2017/065654

§ 371 Date: Jun. 7, 2019

Related U.S. Application Data
(60) Provisional application No. 62/432,526, filed on Dec. 9, 2016.

(51) Int. Cl. A61B 5/00; A61B 5/04; A61B 5/0478; A61B 5/048; A61B 5/16

(52) U.S. Cl. CPC A61B 5/4064; A61B 5/04004; A61B 5/0478; A61B 2505/09; A61B 5/165; A61B 5/4812; A61B 5/4848; A61B 5/048

(57) ABSTRACT

The present invention relates to a method of detecting PTSD in a subject comprising measurement and analysis of brain wave patterns from a subject and determination of a value for one or more neuromarkers from the brain wave pattern. The present invention additionally relates to a system that can be used to diagnose the presence or severity of PTSD in a subject, and to a computer program product for detecting PTSD in a subject by determining if the value of the one or more neuromarkers is above a designated threshold, or is increased or decreased relative to a control value. The invention can also be used to track recovery during and following PTSD therapy, and also as a means for predicting response to therapy and the potential for relapse.

### STATEMENT REGARDING FEDERALLY SPONSORED

This invention was made with government support under grant number B9256-C granted by the VA RR&D Brain Rehabilitation Research Center. The government has certain rights in the invention.

All publications, patents, and patent applications mentioned herein are incorporated by reference to the same extent as if each individual publication, patent or patent application was specifically and individually indicated to be incorporated by reference in its entirety.

### FIELD OF THE INVENTION

The subject matter disclosed herein is generally directed to diagnosis of post-traumatic stress disorder (PTSD). In particular, the invention relates to methods and systems for determining the presence or severity of PTSD.

### BACKGROUND

Post-traumatic stress disorder (PTSD) is a chronic and disabling anxiety disorder that results from exposure to a traumatic event. PTSD is associated with marked deficits in behavioral, social, and occupational functioning. Diagnosis of PTSD is currently established subjectively on the basis of a patient's clinical history, mental status examination, duration of symptoms, and clinician-administered symptom checklists or patient self-reports. However, a recent comprehensive assessment of the current military and veteran PTSD diagnosis and treatment methods by the Institute of Medicine, National Academy of Science recommends that objective physiology-based markers of PTSD would greatly enhance prevention, diagnosis, and treatment success.

### SUMMARY OF THE INVENTION

In one aspect, the invention provides a method for detecting post-traumatic stress disorder in a subject comprising the steps of: obtaining a brain wave pattern from a subject; determining a value for one or more the neuromarkers set forth in Table 3 from the brain wave pattern; detecting post-traumatic stress disorder in the subject by determining if the value of the one or more neuromarkers is above a designated threshold, or is increased or decreased relative to a control value. In one embodiment, the brain wave pattern is obtained from analysis of the subject's brain function during sleep. In another embodiment, the brain function is assessed with the use of polysomnography (PSG). In other embodiments, wherein data from the polysomnography is analyzed in 30, 29, 28, 27, 26, 25, 24, 23, 22, 21, 20, 19, 18, 17, 16, 15, 14, 13, 12, 11, 10, 9, 7, 6, 5, 4, 3, 2, 1, 0.9, 0.8, 0.7, 0.6, 0.5, 0.4, 0.3, 0.2, of 0.1 second intervals. In one example embodiment, data obtained during all or a portion of the polysomnography is analyzed in 5 second intervals. In further embodiments, the polysomnography comprises an electroencephalogram, or comprises performing coherence computation or phase delays. In still further embodiments, the coherence computation is measured between specific electroencephalogram pairs. In some specific embodiments of the invention, polysomnography analyzes a stage of sleep selected from the group consisting of rapid eye movement sleep, stage 1, stage 2, and stage 3. Some embodiments provide polysomnography wherein one or more measurements are selected from the group consisting of presence or absence of a particular sleep stage, frequency of occurrence of a particular sleep stage, sequence of occurrence of a particular sleep stage, degree of sleep fragmentation, and fluctuation patterns across sleep stages. In other embodiments, the subject does not exhibit symptoms of post-traumatic stress disorder, or the method further comprises performing at least one diagnostic analysis selected from the group consisting of an electro-oculogram and a chin electromyogram.

In another aspect, the invention provides a method for determining the severity of post-traumatic stress disorder in a subject, comprising the steps of: performing a sleep analysis on the subject to obtain data; analyzing the data obtained from the sleep analysis to determine the levels of one or more markers selected from the group consisting of the markers set forth in Table 17. In one embodiment, the method further comprises at least one diagnostic criteria selected from the group consisting of clinical history, mental status examination, duration of symptoms, clinician-administered symptom checklist, and patient self-report. In another method, the data obtained is correlated to additional factors selected from the group consisting of persistent nightmares, severe nightmares, sleep disturbances, insomnia, poor daytime functioning, fatigue, mood disorders, and depression.

In another aspect, the invention provides a method of identifying a marker for post-traumatic stress disorder comprising the steps of: analyzing the brain wave pattern of a subject during sleep; and identifying a marker comprising altered levels in a subject having post-traumatic stress disorder compared to a subject lacking post-traumatic stress disorder. In one embodiment, analyzing the brain wave pattern comprises the steps of: analysis of the macro-structure of sleep of the subject by polysomnography; and analysis of the micro-structure of sleep of the subject by electroencephalogram. In another embodiment, the macro-structure is analyzed by obtaining measurements for at least one variable selected from the group consisting of total sleep time, sleep latency, wake after sleep onset, sleep efficiency, the fraction of sleep spent in stage I, the fraction of sleep spent in stage 2, the fraction of sleep spent in stage 3, and the fraction of sleep spent in rapid eye movement sleep. In other embodiments, the micro-structure is analyzed by obtaining measurements of inter-hemispheric and intra-hemispheric coherences and phase delays, or the inter-hemispheric and intra-hemispheric coherences and phase delays comprise measurement of transition between sleep stages. In a further embodiment, the transitions comprise one or more transition type selected from the group consisting of SI to S2, S1 to REM, S2 to S1, and REM to S1.

In another aspect, the invention provides a system for detecting PTSD, comprising: an electroencephalogram (EEG) device for measuring brain wave function of a subject; a storage device; and a processor communicatively coupled to the storage device, wherein the processor executes application code instructions that are stored in the storage device and that cause the system to: obtain a brain wave pattern from the EEG device; determine a value for one or more of the neuromarkers of Table 3; and detect post-traumatic stress disorder in the subject by determining if the value of the one or more neuromarkers is above a designated threshold, or is increased or decreased relative to a control value. In one embodiment, the brain wave pattern is obtained from analysis of the subject's brain function during sleep. In another embodiment, the brain function is assessed with the use of polysomnography. In additional embodiments, data obtained during polysomnography is obtained every 5 seconds.

In still further embodiments, the polysomnography comprises an EEG or comprises performing coherence computation or phase delays. In some embodiments, the coherence computation is measured between specific EEG pairs (Jasper, H. H. (1958). The ten twenty electrode system of the international federation. Electroenceph. and Clinical Neurophysiology, 10, 371-375). In other embodiments, the polysomnography analyzes a stage of sleep selected from the group consisting of REM sleep, stage I, stage 2, and stage 3. In further embodiments, the polysomnography provides one or more measurement selected from the group consisting of presence or absence of a particular sleep stage, frequency of occurrence of a sleep stage, sequence of occurrence of a particular sleep stage, degree of sleep fragmentation, and fluctuation patterns across sleep stages. In one embodiment, the subject does not exhibit symptoms of PTSD.

In other embodiments, the application code instructions that are stored in the storage device further cause the system to correlate the value of the one or more neuromarkers to data obtained from at least one diagnostic analysis selected from the group consisting of an electro-oculogram and a chin electromyogram. In another embodiment, the application code instructions that are stored in the storage device further cause the system to correlate the value of the one or more neuromarkers to data obtained from at least one diagnostic criteria selected from the group consisting of clinical history, mental status examination, duration of symptoms, clinician-administered symptom checklist, and patient self-report. In a still further embodiment, the application code instructions that are stored in the storage device further cause the system to correlate the value of the one or more neuromarkers to additional factors selected from the group consisting of persistent nightmares, severe nightmares, sleep disturbances, insomnia, poor daytime functioning, fatigue, mood disorders, and depression.

In another aspect, the invention provides a computer program product, comprising: a non-transitory computer-executable storage device having computer-readable instructions embodied thereon that when executed by a computer to detect post-traumatic stress disorder in subjects, the computer-executable program instructions comprising: computer-executable program instructions to receive a brain wave pattern; computer-executable program instructions to determine a value for one or more of the neuromarkers set forth in Table 17 from the brain wave pattern; and computer-executable programs instructions to detect post-traumatic stress disorder in the subject by determining if the value of the one or more neuromarkers is above a designated threshold, or is increased or decreased relative to a control value.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

FIG. 1 shows examples of sleep architecture in normal subjects and PTSD patients evaluated for preliminary studies.

FIG. 2A-F shows a strong association between PTSD neuromarkers and the standard PTSD checklist (PCL) from seven PTSD patients. Elements of PTSD neuromarkers associated with coherences between the frontal and central sites of the right hemisphere, in sigma band and during stages I and 2 sleep, exhibit very strong correlations coefficients (Pearson) with PCL.

FIG. 3 shows a block flow diagram depicting a method to detect PTSD, in accordance with certain example embodiments.

FIG. 4 shows a block diagram depicting a computing machine and a module, in accordance with certain example embodiments.

FIG. 5 shows the standard clinical sleep EEG locations according the International 10-20 EEG placement sites (Jasper, H. H. (1958). The ten twenty electrode system of the international federation. Electroenceph. and Clinical Neurophysiology, 10, 371-375). The EEG Montage for the sleep study consisted of the following leads: Frontal: F3 and F4; Central: C3 and C4, Occipital O1 and O2.

FIG. 6A-F show graphically the 6 markers that are included in the PTSD_Diag_Wake Neuromarker. For each marker, the figure shows the two pair of EEG electrodes whose coherence were utilized to calculate the maker. For example, in FIG. 6A, Marker_1 is computed from the coherence between O1-O2 at 0.6 Hz divided by the coherence between O1-F4 computed at 2.4 Hz.

FIG. 7 shows a box plot comparison of PTSD_Diag Neuromarker from awake state between the control and PTSD groups. On each box, the central mark is the median, the edges of the box are the 25th and 75th percentiles, the whiskers extend to the most extreme data-points the algorithm considers to be not outliers, and the outliers are plotted individually. ANOVA showed that the mean of the neuromarker of PTSD is significantly larger than the control group (sig. at F=276, p<10\hat{ }⁻⁸).

FIG. 8 shows the scatter plot and regression line of PTSD_Symptom_Wake vs PCL-5. PTSD_Symptom_Wake was computed from awake state before sleep initiation, the ratio of coherence between C3 and F4 (@ 20 Hz.) divided by the coherence between O2 and C3 (@ 6.8 Hz) The relationship between the Neuromarker and PCL-5 is significant with an R² of 0.6 (F=42, p<10⁻⁶).

FIG. 9A-G show graphically the seven markers that are included in the PTSD_Symptom_Wake Neuromarker. For each marker, the figure shows the two pair of EEG electrodes whose coherence were utilized to calculate the maker. For example, in FIG. 9A, Marker_1 is computed from the coherence between O1-C3 pair at 8.4 Hz divided by the coherence between O2-F3 computed at 2.8 Hz.

FIG. 10 shows the scatter plot and regression line of PTSD_Symptom_Wake vs PCL-5. The PTSD_Symptom_Wake was computed based on the combinations of the seven markers shown in FIG. 9. The relationship between the Neuromarker and PCL-5 is strong and significant with an R² of 0.85 (F=208, p<10⁻⁶).

FIG. 11A-B shows graphically the 2 markers that are included in the PTSD_Diag_Wake Neuromarker using a 1 Hz frequency band-width for computing the coherence values.

FIG. 12 is a box plot comparison of PTSD_Diag_Awake Neuromarker from Awake state between the control and PTSD groups based on coherence calculation using a 1-Hz frequency band-width. On each box, the central mark is the median, the edges of the box are the 25th and 75th percentiles, the whiskers extend to the most extreme data-points the algorithm considers to be not outliers, and the outliers are plotted individually. ANOVA showed that the mean of the neuromarker of PTSD is significantly larger than the control group (sig. at F=367, p<10\hat{ }⁻⁸).

FIG. 13A-G show graphically the seven markers that are included in the PTSD_Symptom_Wake Neuromarker based on coherence calculation using a 1-Hz frequency band-width. For each marker, the figure shows the two pair of EEG electrodes whose coherence were utilized to calculate the maker. For example, in FIG. 13-A, Marker_1 is computed from the coherence between O1-C3 pair in the 8.4-9.4 Hz frequency band, divided by the coherence between O2-F4 computed in the 12.6-13.6 Hz band.

FIG. 14 shows the scatter plot and regression line of PTSD_Symptom_Wake (based on the seven markers using 1-Hz frequency band-width) vs PCL-5. The relationship between the Neuromarker and PCL-5 was strong and significant with an R² of 0.79 (F=140, p<10⁻⁶).

FIG. 15A-L. FIG. 15A-J shows graphically the 10 individual markers that are combined to produce the diagnostic PTSD_Diag_S2 computed during Stage 2 sleep. For each marker, the figures show the two pair of EEG electrodes whose coherence were utilized to calculate the maker. For example, in FIG. 15A, Marker_1 is computed from the coherence between O1-O2 pair at 6.2 Hz divided by the coherence between C3-C4 computed at 7.0 Hz.

FIG. 15K is a box plot comparison of PTSD_Diag_S2 Neuromarker, computed from the ten markers of FIG. 15A-J (Stage 2 sleep) between the control and PTSD groups. On each box, the central mark is the median, the edges of the box are the 25th and 75th percentiles, the whiskers extend to the most extreme data-points the algorithm considers to be not outliers, and the outliers are plotted individually. ANOVA showed that the mean of the neuromarker of PTSD is significantly larger than the control group (sig. at F=1803, p<10\hat{ }⁻⁸). FIG. 15L shows the scatter plot and regression line of PTSD_Symptom_S2 vs PCL-5. PTSD_Symptom_S2 Neuromarker is computed from the ratio of coherence between C3 and F4 (@ 19.6 Hz.) divided by the coherence between O2 and F3 (@ 10.0 Hz). The relationship between the Neuromarker and PCL-5 is significant with an R² of 0.47 (F=31 p<10⁻⁵).

FIG. 16A-E show graphically the 5 markers that are included in the PTSD_Symptom_S2 Neuromarker computed during Stage 2 sleep. For each marker, the figure shows the two pair of EEG electrodes whose coherence were utilized to calculate the maker. For example, in FIG. 16-A, Marker_1 is computed from the coherence between O1-C3 pair at 0.6 Hz divided by the coherence between O2-C4 computed at 1.0 Hz.

FIG. 17 shows the plot and regression line of PTSD_Symptom_S2 vs PCL-5. This PTSD_Symptom_S2 is computed based on the combinations of the five markers shown in FIG. 16. The relationship between the Neuromarker and PCL-5 is strong and significant with an R² of 0.73 (F=96 p<10⁻⁶).

FIG. 18A-F show graphically the 6 markers that are included in the diagnostic Neuromarker PTSD_Diag_S2, computed in Stage 2 sleep and based on coherence calculation using a 1-Hz frequency band-width. For each marker, the figure shows the two pair of EEG electrodes whose coherence were utilized to calculate the maker. For example, in FIG. 18-A, Marker_1 is computed from the coherence between O1-O2 pair in the 6.2-7.2 Hz frequency band, divided by the coherence between C3-C4 in the 7-8 Hz band.

FIG. 19 is a box plot comparison of PTSD_Diag_S2 Neuromarker from Stage 2 sleep, based on the combination of the markers of FIG. 18 and using 1-Hz frequency band-width, between the control and PTSD groups. On each box, the central mark is the median, the edges of the box are the 25th and 75th percentiles, the whiskers extend to the most extreme data-points the algorithm considers to be not outliers, and the outliers are plotted individually. ANOVA showed that the mean of the neuromarker of PTSD is significantly larger than the control group (sig. at F=925, p<10\hat{ }⁻⁸).

FIG. 20A-E. Figure A-D shows graphically the 4 markers that are combined to produce symptom severity PTSD_Symptom_S2 computed during Stage 2 sleep and using 1 Hz bandwidth in the coherence analysis. FIG. 20E shows the scatter plot and regression line of PTSD_Symptom_S2, computed from the markers of FIG. 20A-D vs PCL-5. The relationship between the Neuromarker and PCL-5 is significant with R² of 0.53 (F=41, p<10⁻⁶).

FIG. 21 is a box plot comparison of a combined awake and sleep PTSD diagnostic Neuromarker, computed from the product of PTSD_Diag_S2 and PTSD_Diag_Awake in each individual and based on single frequency coherence analysis, between the control and PTSD groups. On each box, the central mark is the median, the edges of the box are the 25th and 75th percentiles, the whiskers extend to the most extreme data-points the algorithm considers to be not outliers, and the outliers are plotted individually. ANOVA showed that the mean of the neuromarker of PTSD is significantly larger than the control group (sig. at F=962, p<10\hat{ }⁻⁸).

FIG. 22 shows the scatter plot and regression line of the combined awake and sleep neuromarker, PTSD_Symptom_S2×PTSD_Symptom_Awake (single frequency analysis) vs. PCL-5. The relationship between the Neuromarker and PCL-5 was strong and significant with R² of 0.85 (F=199 p<10⁻⁶).

FIG. 23 is a box plot comparison of a combined awake and sleep PTSD diagnostic Neuromarker, computed from the product of PTSD_Diag_S2 and PTSD_Diag_Awake in each individual and based on 1-Hz frequency band-width coherence analysis, between the control and PTSD groups. On each box, the central mark is the median, the edges of the box are the 25th and 75th percentiles, the whiskers extend to the most extreme data-points the algorithm considers to be not outliers, and the outliers are plotted individually. ANOVA showed that the mean of the neuromarker of PTSD was significantly larger than the control group (sig. at F=933, p<10\hat{ }⁻⁸).

FIG. 24 shows the scatter plot and regression line of the combined awake and sleep neuromarker, PTSD_Symptom_S2×PTSD_Symptom_Awake (1 Hz band analysis) vs. PCL-5. The relationship between the Neuromarker and PCL-5 is strong and significant with R² of 0.72 (F=94 p<10⁻⁷).

FIG. 25. The top two tables in the figure show the means, standard deviations, and the medians for the individual markers in the Controls and PTSD group during awake period and at a single frequency. These markers are combined to produce the overall PTSD diagnostic marker, PTSD_Diag_Awake. The bottom table shows similar information about the individual markers that are combined to produce the overall PTSD symptom marker, PTSD_Symp_Awake in the PTSD group.

FIG. 26. The top two tables in the figure show the means, standard deviations, and the medians for the individual markers in the Controls and PTSD group during Stage 2 sleep and at a single frequency. These markers are combined to produce the overall PTSD diagnostic marker, PTSD_Diag_S2. The bottom table shows similar information about the individual markers that are combined to produce the overall PTSD symptom marker, PTSD_Symp_S2 in the PTSD group

FIG. 27. The top two tables in the figure show the means, standard deviations, and the medians for the individual markers in the Controls and PTSD group during awake period and using 1-Hz frequency bands. These markers are combined to produce the overall PTSD diagnostic marker, PTSD_Diag_Awake. The bottom table shows similar information about the individual markers that are combined to produce the overall PTSD symptom marker, PTSD_Symp_Awake in the PTSD group

FIG. 28. The top two tables in the figure show the means, standard deviations, and the medians for the individual markers in the Controls and PTSD group during Stage 2 sleep and using 1-Hz frequency bands. These markers are combined to produce the overall PTSD diagnostic marker, PTSD_Diag_S2. The bottom table shows similar information about the individual markers that are combined to produce the overall PTSD symptom marker, PTSD_Symp_S2 in the PTSD group.

FIG. 29A-B. FIG. 29A shows the EEG electrode pairs for each of the individual markers during awake period, computed at a single frequency, that are combined to produce the overall PTSD_Diag_Awake marker. FIG. 29-B shows the mean±std for each of the six markers for the two groups (controls and PTSD) along with the p-value of the student-t comparison of the means (significant p-values shown in bold).

FIG. 30A-B. FIG. 30-A shows the EEG electrode pairs for each of the individual markers during Stage 2 sleep, computed at a single frequency, that are combined to produce the overall PTSD_Diag_S2 marker. FIG. 30-B shows the mean±std for each of the ten markers for the two groups (controls and PTSD) along with the p-value of the student-t comparison of the means (significant p-values shown in bold).

FIG. 31A-B. FIG. 31-A shows the EEG electrode pairs for each of the individual markers during Awake period, computed using 1-Hz frequency bands, which are combined to produce the overall PTSD_Diag_Awake marker. FIG. 31-B shows the mean±std for each of the two markers for the two groups (controls and PTSD) along with the p-value of the student-t comparison of the means (significant p-values shown in bold).

FIG. 32A-B. FIG. 32-A shows the EEG electrode pairs for each of the individual markers during Stage 2 sleep, computed using 1-Hz frequency bands, which are combined to produce the overall PTSD_Diag_S2 marker. FIG. 32-B shows the mean±std for each of the six markers for the two groups (controls and PTSD) along with the p-value of the student-t comparison of the means (significant p-values shown in bold).

FIG. 33A-E. FIG. 33A-E shows graphically the 5 markers that are included in the PTSD_Diag_REM neuromarker.

FIG. 34. FIG. 34 is a box plot showing PTSD_Diag_REM neuromarker.

FIG. 35A-B. FIG. 35A-B shows graphically 2 markers.

FIG. 36. FIG. 36 is a scatter plot and regression lines of neuromarkers associated with symptom severity of PTSD using REM sleep.

### DETAILED DESCRIPTION OF THE INVENTION

The present invention provides methods for detecting and/or determining the severity of post-traumatic stress disorder (PTSD) in a patient or subject comprising the steps of: obtaining a brain wave pattern and/or electrical activity from a subject; determining a value for one or more the neuromarkers set forth herein from the brain wave pattern; detecting PTSD in the subject by determining if the value of the one or more neuromarkers is above a designated threshold, or is increased or decreased relative to a control value. In accordance with the practice of the invention, PTSD symptoms may be defined or includes following four clusters: Presence of one (or more) of intrusion symptoms associated with the traumatic event; Persistent avoidance of stimuli associated with the traumatic event; Negative alterations in cognition and mood associated with the traumatic event; and Marked alterations in arousal and reactivity associated with the traumatic event (DSM-5 (2013): Diagnostic and Statistical Manual of Mental Disorders (Fifth ed.). American psychiatric Association. Arlington, Va.: American Psychiatric Publishing).

The invention is based on discovery of physiology-based markers of PTSD that are derived from the analysis of neural connectivity between brain hemispheres and lobes during awake and specific stages of sleep. These markers can be utilized for more accurate diagnosis of PTSD, and for objective tracking of treatment outcome and recovery. The invention also provides a system for detecting PTSD, comprising: an electroencephalogram (EEG) device for measuring brain wave activity and function of a subject; a storage device; and a processor communicatively coupled to the storage device, wherein the processor executes application code instructions that are stored in the storage device and that cause the system to: obtain a brain wave pattern from the EEG device; determine a value for one or more of the neuromarkers described herein; and detect post-traumatic stress disorder in the subject by determining if the value of the one or more neuromarkers is above a designated threshold, or is increased or decreased relative to a control value.

In some embodiments, additional diagnostic criteria may be combined with the methods and systems of the invention. For example, clinical history, mental status examination, duration of symptoms, clinician-administered symptom checklist, and patient self-report. Other types of data may also be used in accordance with the invention, including, but not limited to, the presence or absence of persistent and/or severe nightmares, sleep disturbances, insomnia, poor daytime functioning, fatigue, mood disorders, and/or depression. PTSD is a type of anxiety disorder resulting from exposure to a traumatic event, and can include events that result in milder types of traumatic brain injury (mTBI with relatively short periods of concussion and amnesia). PTSD is characterized by subjective-related symptoms including avoidance behaviors, hyper arousal, and re-experiencing symptoms following exposure to the traumatic event [1]. Epidemiologic studies have shown that during their life-time, nearly 56% of people will experience a psychologically traumatic event, of which between 8-12% will develop criteria for PTSD [2, 3]. The risk of developing PTSD is higher for U.S. military veterans than for the general population. The lifetime prevalence of PTSD among Vietnam veterans is estimated to be 19% [4], and similar patterns are observed among soldiers and veterans of Iraq and Afghanistan wars Operation Enduring Freedom (OEF) and Operation Iraqi Freedom (OIF). Approximately 17% of active duty soldiers and 25% of reserve soldiers from OEF met criteria for PTSD three to six months post-deployment [5].

A number of brain imaging studies have reported altered brain activity and connectivity in PTSD in the ventromedial prefrontal cortex (vmPFC), insula, amygdala, and hippocampus. PTSD is also associated with a decrease in functional connectivity (FC) among several brain regions. Diminished levels of connectivity have been found between the posterior cingulate cortex and the right frontal cortex, as well as the left thalamus among PTSD patients. Other studies have reported decreases in the rostral anterior cingulate cortex/vmPFC and an increase in the salience network including the amygdala, during resting state MRI study. Significantly different activity and synchronous neural interactions in PTSD patients compared with normal healthy subjects were reported in a study using magneto-encephalography (MEG). In another study with MEG in a task-free rest state, significant alteration in synchronous correlations were reported between the parietal, temporal, and central regions in PTSD compared to a normal control group. A more recent study using electroencephalography (EEG) in rest state reported that functional connectivity of PTSD patients showed decreased resting-state FC compared to control group, and these FC measures were significantly correlated with PTSD symptom severity.

Diagnosis for PTSD is currently established on the basis of a patient's clinical history, mental status examination, duration of symptoms, and clinician-administered symptom checklists or patient self-reports. The most common treatment for PTSD is prolonged exposure (PE) therapy [6], which is an exposure-based form of cognitive behavioral therapy focused on reducing PTSD and related psychopathology [7]. PE includes components of psycho-education, in vivo exposure to feared, but safe, trauma-related stimuli, imagined exposure, and processing of trauma memories. Fear extinction mechanisms are thought to be the basis of PE success, allowing the patient to emotionally engage and process the traumatic memories in the absence of feared outcomes [6]. Numerous studies have reported successful use of PE for PTSD patients, and the treatment is recommended world-wide in official PTSD treatment guidelines such as the International Society for Traumatic Stress Studies [10], the National Institute for Health and Clinical Excellence Guidelines on PTSD [11]. However, some individuals do not respond to PE, and some respond with only partial recovery to the standard treatment. A comprehensive analysis of the current military and veteran PTSD diagnosis and treatment methods by the Institute of Medicine, National Academy of Science (IMNAS) identified an urgent need for measures that can more accurately diagnose and measure treatment response.

The present invention therefore provides for the first time methods and markers for more precise and objective diagnosis of PTSD and its severity level, for tracking recovery during and following PTSD therapy, as a means for predicting response to therapy and the potential for relapse, for accurate selection of specific evidence-based treatments, objective and faster evaluation of treatment efficacy. The methods and markers are based on the analysis of electroencephalogram (EEG), activity, connectivity, and coupling between various locations on the scalp and forehead of a patient or subject during awake, drowsy, and sleep states (based on standard clinical sleep staging, or a continuum of depth-of-sleep measurement with periods lasting milliseconds to a few seconds) (Jasper, H. H. (1958). The ten twenty electrode system of the international federation. Electroenceph. and Clinical Neurophysiology, 10, 371-375). The invention allows for rapid adjustment of treatment to improve efficacy, or adjustment of dosage selection based on initial response. The neuromarkers described herein also enable prediction of individuals at risk for relapse. In order of a physiology-biology marker to be valuable for diagnosis of PTSD, it would require excellent sensitivity and specificity in distinguishing those with PTSD from others [12]. Similarly, in order to accurately match PTSD patients with specific treatments, a biomarker would require sensitivity to treatment response, such as PE treatment [12].

The preferred mode of analysis in accordance with the present invention s computation of coherence function between various EEG sites producing measurements or values related to the strength of coupling and association between EEG pairs (coherence), as well as their phase relationship (time leads or lags). These quantities are computed during states of the brain including awake, drowsy, and sleep states, determined based on standard clinical sleep staging, or a continuum of depth-of-sleep measurement, to produce multi-dimensional arrays of neuromarkers reflecting synchronicity and phase delays between various EEG sites. EEG is obtained from awake and sleeps periods from a standard overnight sleep study, or from a daytime nap study, performed at home or a hotel/motel room, in an office or examination room, and within hospitals and clinics including sleep laboratories.

The methods of the invention may also be applied to add the analysis of electro-occulogram (EOG) for capturing eye movement, as well as lower prefrontal cortex brain activity. Furthermore, analysis of EEG and EOG coherence functions during awake and sleep states, may be applied to other psychiatric and neurological and neurodegenerative disorders, including, but not limited to depression, anxiety, insomnia, attention deficit/hyperactivity disorder, dementia, Alzheimer's, traumatic brain injury (TBI), depth-of-anesthesia, REM behavioral disorder, Parkinson's, and ALS.

In one embodiment of the invention for detecting PTSD in a subject, the method comprises the following steps. The method comprises obtaining two or more brain wave patterns from at least two locations selected on a head of a subject. As used herein, head includes the face or portions of the face, such as the forehead, temple, around the cheeks or cheekbones; scalp; the area behind the ears; the area under the chin; neck area such as back of the neck. The method further comprises segmenting the brain wave patterns by sleep stage and segmenting the brain wave patterns for one sleep stage so segmented from b into defined time intervals so as to permit auto and cross spectral analysis and/or coherence analysis.

The method further comprises calculating coherence value(s) and/or phase delay value(s) from two brain wave segments for a single frequency or a frequency band. For example, coherence is a generalization of correlation analysis and is computed as the magnitude of normalized cross-power spectrum of a pair of simultaneously recorded electroencephalography (EEG) from two separate head locations (e.g. scalp). In accordance with the practice of the invention, coherence reflects the degree of coupling and functional association between two brain areas. EEG involves electro-physiological monitoring methods that records electrical activity of the brain with the electrodes placed along the head, e.g., the subject's scalp. In a preferred embodiment, in an oscillatory signal, frequency is the number of cycles completed in one second (unit is Hz or cycles/second). In another preferred embodiment, frequency band is a range of frequencies (e.g. 8-12 Hz) and width of a frequency band is the difference of the highest and lowest frequencies within a range of frequencies (e.g., 8-12 Hz frequency band has a 4 Hz width). In a preferred embodiment, the width of a frequency band is 1 Hz. In a preferred embodiment, the frequency band comprises or is in the proximity (e.g., within 5 Hz) of a single frequency marker useful in defining a neuromarker.

The method further comprises determining whether the coherence value(s), phase delay value(s) and/or a combination thereof is above or below a designated threshold so as to determine presence of PTSD in the subject thereby, detecting post-traumatic stress disorder in the subject.

In a further embodiment of the invention, the combination thereof of coherence value(s) and/or phase delay values is multiple linear regression of coherence value(s) and/or phase delay values.

In another embodiment of the method for detecting post-traumatic stress disorder (PTSD) in a subject, the method comprises a) obtaining two or more brain wave patterns from at least two locations selected on a head of a subject; b) segmenting the brain wave patterns by sleep stage; c) segmenting the brain wave patterns for one sleep stage so segmented from step (b) into defined time intervals so as to permit auto and cross spectral analysis and/or coherence analysis; d) calculating coherence value(s) and/or phase delay value(s) from two brain wave segments of step (c) for a single frequency or a frequency band at a particular sleep stage; and (e) repeating step (d) to obtain more coherence values and/or phase delay values for the same sleep stage, at (i) other single frequency or frequency band obtained from the same two locations, and/or (ii) the same or other single frequency or frequency band obtained from two different locations or two locations in which one location is shared in common in step (d). Optionally, the method further comprises f) performing steps (d) and (e) for a different sleep stage or multiple sleep stages.

Additionally, the method further comprises (g) combining coherence value(s) or phase delay value(s) so as to be a marker or a combination of markers. In a preferred embodiment, the markers so chosen are those that have or possess a coherence ratio at a certain single frequency, or a 1-Hz frequency band, that maximally separated control from PTSD group based on ANOVA analysis.

Also, the method further comprises (h) selecting a neuromarker from the markers or combination of markers of step (g). In one embodiment, the neuromarker may be defined as: (i) a single coherence value ratio or phase delay value ratio; (ii) combination of two or more markers of step (g) for a particular sleep stage or awake period; and/or (iii) combination of two or more markers of step (g) from two or more sleep stages and/or awake period. In a preferred embodiment, neuromarkers were selected based on using a single marker, or using a step-wise linear regression with all of the markers. For example, in a step-wise regression, only the particular markers whose p-values were less than 0.001 were chosen to be included in the regression model. In another embodiment, only particular markers who displayed significant P values such as less than P=0.05; p-values of P=0.01 or less; or p-values of P=0.001 or less were chosen.

The method further comprises (i) determining whether the value of the neuromarker for diagnosing PTSD is above or below a designated threshold, so as to determine presence of PTSD in the subject.

In accordance with the practice of the invention, the mathematical combination of two or more markers in steps (f) and (g) for a particular sleep stage is or comprises a combination of markers using multiple linear regression.

In a further embodiment of the invention, the combination of markers is multiple linear regression of multiple linear regression of markers, respectively.

In one embodiment of the invention, the brain wave patterns may be obtained from analysis of the subject's brain function during sleep, awake-to-sleep and/or awake to sleep initiation. In accordance with the practice of the invention, the brain function may be assessed with the use of polysomnography. Merely by way of example, the polysomnography may comprise electroencephalography (EEG).

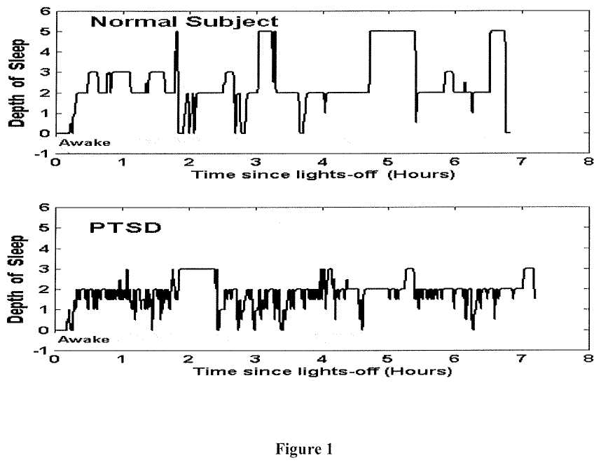

In one embodiment of the method, the EEG comprises placement of at least two EEG electrodes on at least two head or scalp locations of the subject's head. For example, the head or scalp locations may be selected from head or scalp electrode placement locations according to International 10-20 system (Jasper, H. H. (1958). The ten twenty electrode system of the international federation. Electroenceph. and Clinical Neurophysiology, 10, 371-375) or as provided in FIG. 1. In one embodiment, the scalp electrode placement, according to International 10-20 system, includes head or scalp locations, Fp1, Fp2, F3, F4, F7, F8, Fz, A1, A2, C3, C4, Cz, T3, T4, T5, T6, P3, P4, Pz, O1 and O2. In another embodiment, the head or scalp locations include F3, F4, C3, C4, O1 and O2.

Merely by way of example, brain wave patterns may be obtained from six scalp locations using EEG electrodes placed at scalp locations F3, F4, C3, C4, O1 O2, and two reference electrodes placed at A1 and A2, or at the middle of forehead. In one embodiment of the invention, the brain wave patterns are recorded simultaneously.

In accordance with the practice of the invention, brain wave patterns are segmented into sleep stages, and examples of the sleep stages include, but are not limited to, awake period with lights off and before falling asleep (W), stage I sleep, stage II sleep (S2), delta-wave or stable III sleep or rapid-eye-movement (REM) sleep (Berry R B, Brooks R, Gamaldo C E, Harding S M, Marcus C L and Vaughn B V for the American Academy of Sleep Medicine. The AASM Manual for the Scoring of Sleep and Associated Events: Rules, Terminology and Technical Specifications, Version 2.2. aasm.org).

In one embodiment of the method, the particular sleep stage includes, but is not limited to, any of awake period including the period with lights off and before falling asleep (W), stage I sleep, stage II sleep (S2), delta-wave or stable III sleep, and rapid-eye-movement (REM) sleep. In another embodiment, the particular sleep stage is either awake period with lights off and before falling asleep (W) or stage II sleep (S2). In a further embodiment, the particular sleep stage is awake period with lights off and before falling asleep (W). In yet a further embodiment, the particular sleep stage is stage II sleep (S2).

In an embodiment of the invention, the defined time period so measure at a particular sleep stage may be greater than 2 seconds and less than 30 seconds. For example, the defined time period so measure at a particular sleep stage may be about 5 seconds.

In accordance with the practice of the invention, the single frequency includes, but is not limited to, any frequency between 0 Hz and 52 Hz. For example, the single frequency includes, but is not limited to, about 0.2, 0.4, 0.6, 0.8, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 12.2, 12.4, 12.6, 12.8, 13.0, 13.2, 13.4, 13.6, 13.8, 14.0, 14.2, 14.4, 14.6, 14.8, 15.0, 15.2, 15.4, 15.6, 15.8, 16.0, 16.2, 16.4, 16.6, 16.8, 17.0, 17.2, 17.4, 17.6, 17.8, 18.0, 18.2, 18.4, 18.6, 18.8, 19.0, 19.2, 19.4, 19.6, 19.8, 20.0, 20.2, 20.4, 20.6, 20.8, 21.0, 21.2, 21.4, 21.6, 21.8, 22.0, 22.2, 22.4, 22.6, 22.8, 23.0, 23.2, 23.4, 23.6, 23.8, 24.0, 24.2, 24.4, 24.6, 24.8, 25.0, 25.2, 25.4, 25.6, 25.8, 26.0, 26.2, 26.4, 26.6, 26.8, 27.0, 27.2, 27.4, 27.6, 27.8, 28.0, 28.2, 28.4, 28.6, 28.8, 29.0, 29.2, 29.4, 29.6, 29.8, 30.0, 30.2, 30.4, 30.6, 30.8, 33.0, 33.2, 33.4, 33.6, 33.8, 32.0, 32.2, 32.4, 32.6, 32.8, 33.0, 33.2, 33.4, 33.6, 33.8, 34.0, 34.2, 34.4, 34.6, 34.8, 35.0, 35.2, 35.4, 35.6, 35.8, 36.0, 36.2, 36.4, 36.6, 36.8, 37.0, 37.2, 37.4, 37.6, 37.8, 38.0, 38.2, 38.4, 38.6, 38.8, 39.0, 39.2, 39.4, 39.6, 39.8, 40.0, 40.2, 40.4, 40.6, 40.8, 44.0, 44.2, 44.4, 44.6, 44.8, 42.0, 42.2, 42.4, 42.6, 42.8, 43.0, 43.2, 43.4, 43.6, 43.8, 44.0, 44.2, 44.4, 44.6, 44.8, 45.0, 45.2, 45.4, 45.6, 45.8, 46.0, 46.2, 46.4, 46.6, 46.8, 47.0, 47.2, 47.4, 47.6, 47.8, 48.0, 48.2, 48.4, 48.6, 48.8, 49.0, 49.2, 49.4, 49.6, 49.8, 50.0, 50.2, 50.4, 50.6, 50.8, 51.0, 51.2, 51.4, 51.6, 51.8 and/or 52.0 Hz. In accordance with the practice of the invention, the single frequency may vary by ±0.1 Hz.

In another embodiment, the single frequency includes, but is not limited to, about 0.6, 2.4, 2.6, 3.2, 4.2, 6.2, 6.6, 6.8, 7.0, 7.4, 7.6, 8.6, 11.0, 11.2, 11.8, 13.0, 13.4, 15.8, 16.4, 16.6, 16.8, 23.8, 37.0 and/or 41.4 Hz. In a further embodiment, the single frequency includes, but is not limited to, about 0.6, 2.4, 4.2, 6.8, 7.4, 7.6, 8.6, 13.0, 16.8, 23.8 and 37.0 Hz for the sleep stage corresponding to awake period with lights off and before falling asleep (W). In yet a further embodiment, the single frequency includes, but is not limited to, about 0.6, 2.6, 3.2, 6.2, 6.6, 6.8, 7.0, 11.0, 11.2, 11.8, 13.4, 15.8, 16.4, 16.6 and 41.4 Hz for the sleep stage corresponding to stage II sleep (S2).

In accordance with the practice of the invention, the single frequency may vary by any value within an about 2 Hz window. For example, the single frequency may vary by any value within an about 0.2 Hz window. In another example, the single frequency may vary by any value within an about 0.1 Hz window.

In an embodiment of the invention, the frequency band may be about and comprises the frequency including any of 0.6, 2.4, 2.6, 3.2, 4.2, 6.2, 6.6, 6.8, 7.0, 7.4, 7.6, 8.6, 11.0, 11.2, 11.8, 13.0, 13.4, 15.8, 16.4, 16.6, 16.8, 23.8, 37.0 and 41.4 Hz. In another embodiment of the invention, the frequency band may be about and comprises the frequency selected from the group consisting of 0.6, 2.4, 4.2, 6.8, 7.4, 7.6, 8.6, 13.0, 16.8, 23.8 and 37.0 Hz for the sleep stage corresponding to awake period with lights off and before falling asleep (W). In a further embodiment of the invention, the frequency band may be about and comprises the frequency selected from the group consisting of 0.6, 2.6, 3.2, 6.2, 6.6, 6.8, 7.0, 11.0, 11.2, 11.8, 13.4, 15.8, 16.4, 16.6 and 41.4 Hz for the sleep stage corresponding to stage II sleep (S2).

Merely by way of example, the width of the frequency band may be at least a 0.1 Hz but less than 20 Hz. In an embodiment of the invention, the width of the frequency band is least a 0.2 Hz but less than 10 Hz. In another embodiment of the invention, the width of the frequency band includes, but is not limited to, any of 0.1 Hz, 0.2 Hz, 0.5 Hz, 1 Hz, 1.5 Hz, 2 Hz, 2.5 Hz, 3 Hz, 3.5 Hz, 4 Hz, 4.5 Hz, 5 Hz, 5.5 Hz, 6 Hz, 6.5 Hz, 7 Hz, 7.5 Hz, 8 Hz, 8.5 Hz, 9 Hz, 9.5 Hz and 10 Hz. In a specific embodiment, the width of the frequency band is about 1 Hz. In another particular embodiment, the width of the frequency band is 1 Hz selected over the frequency between 0-52 Hz.

Merely by way of example, the frequency band includes, but is not limited to, any of 0.2-1.2, 0.4-1.4, 0.6-1.6, 0.8-1.8, 1.0-2.0, 1.2-2.2, 2.4-3.4, 2.6-3.6, 2.8-3.8, 3.0-4.0, 3.2-4.2, 3.4-4.4, 3.6-4.6, 3.8-4.8, 4.0-5.0, 4.2-5.2, 4.4-5.4, 4.6-5.6, 4.8-5.8, 5.0-6.0, 5.2-6.2, 5.4-6.4, 5.6-6.6, 5.8-6.8, 6.0-7.0, 6.2-7.2, 6.4-7.4, 6.6-7.6, 6.8-7.8, 7.0-8.0, 7.2-8.2, 7.4-8.4, 7.6-8.6, 7.8-8.8, 8.0-9.0, 8.2-9.2, 8.4-9.4, 8.6-9.6, 8.8-9.8, 9.0-10.0, 9.2-10.2, 9.4-10.4, 9.6-10.6, 9.8-10.8, 10.0-11.0, 10.2-11.2, 10.4-11.4, 10.6-11.6, 10.8-11.8, 11.0-12.0, 11.2-12.2, 11.4-12.4, 11.6-12.6, 11.8-12.8, 12.0-13.0, 12.2-13.2, 12.4-13.4, 12.6-13.6, 12.8-13.8, 13.0-14.0, 13.2-14.2, 13.4-14.4, 13.6-14.6, 13.8-14.8, 14.0-15.0, 15.2-16.2, 15.4-16.4, 15.6-16.6, 15.8-16.8, 16.0-17.0, 16.2-17.2, 16.4-17.4, 16.6-17.6, 16.8-17.8, 17.0-18.0, 17.2-18.2, 17.4-18.4, 17.6-18.6, 17.8-18.8, 18.0-19.0, 18.2-19.2, 18.4-19.4, 18.6-19.6, 18.8-19.8, 19.0-20.0, 19.2-20.2, 19.4-20.4, 19.6-20.6, 19.8-20.8, 20.0-21.0, 20.2-21.2, 20.4-21.4, 20.6-21.6, 20.8-21.8, 21.0-22.0, 21.2-22.2, 21.4-22.4, 21.6-22.6, 21.8-22.8, 22.0-23.0, 22.2-23.2, 22.4-23.4, 22.6-23.6, 22.8-23.8, 23.0-24.0, 23.2-24.2, 23.4-24.4, 23.6-24.6, 23.8-24.8, 24.0-25.0, 24.2-25.2, 24.4-25.4, 24.6-25.6, 24.8-25.8, 25.0-26.0, 25.2-26.2, 25.4-26.4, 25.6-26.6, 25.8-26.8, 26.0-27.0, 26.2-27.2, 26.4-27.4, 26.6-27.6, 26.8-27.8, 27.0-28.0, 27.2-28.2, 27.4-28.4, 27.6-28.6, 27.8-28.8, 28.0-29.0, 28.2-29.2, 28.4-29.4, 28.6-29.6, 28.8-29.8, 29.0-30.0, 29.2-30.2, 29.4-30.4, 29.6-30.6, 29.8-30.8, 30.0-31.0, 30.2-31.2, 30.4-31.4, 30.6-31.6, 30.8-31.8, 31.0-32.0, 31.2-32.2, 31.4-32.4, 31.6-32.6, 31.8-32.8, 32.0-33.0, 32.2-33.2, 32.4-33.4, 32.6-33.6, 32.8-33.8, 33.0-34.0, 33.2-34.2, 33.4-34.4, 33.6-34.6, 33.8-34.8, 34.0-35.0, 34.2-35.2, 34.4-35.4, 34.6-35.6, 34.8-35.8, 35.0-36.0, 35.2-36.2, 35.4-36.4, 35.6-36.6, 35.8-36.8, 36.0-37.0, 36.2-37.2, 36.4-37.4, 36.6-37.6, 36.8-37.8, 37.0-38.0, 37.2-38.2, 37.4-38.4, 37.6-38.6, 37.8-38.8, 38.0-39.0, 38.2-39.2, 38.4-39.4, 38.6-39.6, 38.8-39.8, 39.0-40.0, 39.2-40.2, 39.4-40.4, 39.6-40.6, 39.8-40.8, 40.0-41.0, 40.2-41.2, 40.4-41.4, 40.6-41.6, 40.8-41.8, 41.0-42.0, 41.2-42.2, 41.4-42.4, 41.6-42.6, 41.8-42.8, 42.0-43.0, 42.2-43.2, 42.4-43.4, 42.6-43.6, 42.8-43.8, 43.0-44.0, 43.2-44.2, 43.4-44.4, 43.6-44.6, 43.8-44.8, 44.0-45.0, 44.2-45.2, 44.4-45.4, 44.6-45.6, 44.8-45.8, 45.0-46.0, 46.2-47.2, 46.4-47.4, 46.6-47.6, 46.8-47.8, 47.0-48.0, 47.2-48.2, 47.4-48.4, 47.6-48.6, 47.8-48.8, 48.0-49.0, 48.2-49.2, 48.4-49.4, 48.6-49.6, 48.8-49.8, 49.0-50.0, 49.2-50.2, 49.4-50.4, 49.6-50.6, 49.8-50.8, 50.0-51.0, 50.2-51.2, 50.4-51.4, 50.6-51.6, 50.8-51.8 and 51.0-52.0 Hz. Merely by way of example, the frequency band may start anywhere within the frequency bands described above but end outside of said frequency band range so long as a 1 Hz band width is maintained.

Merely by way of example, the frequency band includes any of 0.6-1.6, 2.6-3.6, 6.2-7.2, 6.8-7.8, 7.0-8.0, 7.4-8.4, 7.6-8.6, 8.6-9.6, 11.0-12.0, 11.2-12.2, 16.4-17.4, 16.6-17.6, 19.4-20.4 and 41.4-42.4 Hz. In one embodiment, the frequency band includes any of 6.8-7.8, 7.4-8.4, 7.6-8.6 and 8.6-9.6 Hz for the sleep stage corresponding to awake period with lights off and before falling asleep (W). In another embodiment, the frequency band includes any of 0.6-1.6, 2.6-3.6, 6.2-7.2, 7.0-8.0, 8.6-9.6, 11.0-12.0, 11.2-12.2, 16.4-17.4, 16.6-17.6, 19.4-20.4 and 41.4-42.4 Hz for the sleep stage corresponding to stage II sleep (S2). In accordance with the practice of the invention, the frequency band may start anywhere within said frequency band but end outside of said frequency band range to maintain a 1 Hz frequency band.

In an embodiment of the invention, the two locations include any of: O1-O2; C3-C4; F3-F4; O1-C3; O2-C4; C3-F3; C4-F4; O1-C4; O2-C3; C3-F4; C4-F3; O1-F3; O1-F4; O2-F3; and O2-F4. In another embodiment, the two locations include any of: O1-O2; C3-C4; F3-F4; O1-C3; O2-C4; C3-F3; C4-F4; O1-C4; O2-C3; C3-F4; O1-F3; O1-F4; and O2-F3. In yet a further embodiment, the two locations are selected from the group consisting of: O1-O2; C3-C4; F3-F4; C3-F3; O2-C3; C3-F4; O1-F4; and O2-F3 for the particular sleep stage corresponding to awake period with lights off and before falling asleep (W). In an additional embodiment, the two locations include any of: O1-O2; C3-C4; O1-C3; O2-C4; C3-F3; C4-F4; O1-C4; O2-C3; C3-F4; O1-F3; and O2-F3 for the for the particular sleep stage corresponding to stage II sleep (S2).

In an embodiment of the invention, the coherence value(s), phase delay value(s) and/or combination thereof, are determine based on brain wave patterns from recordings at two locations as provided herein and at single frequency as provided herein or at frequency band as provided herein claim. In a specific embodiment, the coherence value(s), and/or combination thereof, for the sleep stage corresponding to awake period with lights off and before falling asleep (W) and for single frequency of brain wave patterns recorded at two locations (e.g., scalp locations) includes any of: Coh. O1-O2 (@ 0.6 Hz); Coh. O1-F4 (@ 2.4 Hz); Coh. C3-C4 (@ 23.8 Hz); Coh. O2-C3 (@ 37.0 Hz); Coh. F3-F4 (@8.6 Hz); Coh. C3-F3 (@ 6.8 Hz); Coh. F3-F4 (@ 7.4 Hz); Coh. C3-F4 (@ 7.6 Hz); Coh. F3-F4 (@ 16.8 Hz); Coh. O1-F4 (@ 7.4 Hz); Coh. O1-F4 (@ 4.2 Hz); and Coh. O2-F3 (@ 13.0 Hz); or a combination thereof.

In another embodiment, coherence value(s) and/or combination thereof, for the sleep stage corresponding to awake period with lights off and before falling asleep (W) and for frequency band from brain wave patterns recorded at two locations, e.g., scalp locations, include any of: Coh. F3-F4 (8.6-9.6 Hz); Coh. C3-F3 (@ 6.8-7.8 Hz); Coh. F3-F4 (@ 7.6-8.6 Hz); and Coh. O2-F3 (@ 7.4-8.4 Hz); or a combination thereof. In a further embodiment, the coherence value ratio useful as a marker for the sleep stage corresponding to awake period with lights off and before falling asleep (W) includes any of: Coh. O1-O2 (@ 0.6 Hz)/Coh. O1-F4 (@ 2.4 Hz); Coh. C3-C4 (@ 23.8 Hz)/Coh. O2-C3 (@ 37.0 Hz); Coh. F3-F4 (@ 8.6 Hz)/Coh. C3-F3 (@ 6.8 Hz); Coh. F3-F4 (@ 7.4 Hz)/Coh. C3-F4 (@ 7.6 Hz); Coh. F3-F4 (@ 16.8 Hz)/Coh. O1-F4 (@ 7.4 Hz); and Coh. O1-F4 (@ 4.2 Hz)/Coh. O2-F3 (@ 13.0 Hz); or a combination thereof.

In yet a further embodiment, the coherence value ratio useful as a marker for the sleep stage corresponding to awake period with lights off and before falling asleep (W) includes any of: Coh. F3-F4 (8.6-9.6 Hz)/Coh. C3-F3 (@ 6.8-7.8 Hz); and Coh. F3-F4 (@ 7.6-8.6 Hz)/Coh. O2-F3 (@ 7.4-8.4 Hz); or a combination thereof.

In another embodiment of the method, combining the coherence value(s) or phase delay value(s) is a mathematical operation performed on two or more coherence value(s) or phase delay value(s), wherein the mathematical operation is selected from the group consisting of addition, subtraction, multiplication, division, factorial, sigma, n-th root, exponential, logarithm, mean, median, mode, standard deviation, coefficient of variation, geometric sequence, arithmetic sequence, normalization, binary, averaging, ratiometric, trigonometric function, linear function, exponential function, logarithmic function and function with input of coherence value or phase delay value as a dependent variable, regression function, linear regression, multiple linear regression, logistic regression, polynomial regression, nonlinear regression, nonparametric function and semiparametric function, and a combination thereof.

In another embodiment of the method, combining the coherence value(s) or phase delay value(s) is or comprises dividing one coherence value by a second coherence value or a combination of coherence values so as to obtain a coherence value ratio useful as a marker, dividing one phase delay value by a second phase delay value or a combination of phase delay values so as to obtain a phase delay value ratio useful as a marker, wherein a combination of coherence values or a combination of phase delay values used in the division as a denominator is obtained by performing a mathematical operation selected from the group consisting of addition, subtraction, multiplication, division, factorial, sigma, n-th root, exponential, logarithm, mean, median, mode, standard deviation, coefficient of variation, geometric sequence, arithmetic sequence, normalization, binary, averaging, ratiometric, trigonometric function, linear function, exponential function, logarithmic function, function with input of coherence value or phase delay value as a dependent variable, regression function, linear regression, multiple linear regression, logistic regression, polynomial regression, nonlinear regression, nonparametric function and semiparametric function, and a combination thereof, for a set of coherence values or phase delay values, respectively.

In yet another embodiment of the method, combining the coherence value(s) or phase delay value(s) is or comprises dividing one coherence value by a second coherence value or a combination of coherence values so as to obtain a coherence value ratio useful as a marker, dividing one phase delay value by a second phase delay value or a combination of phase delay values so as to obtain a phase delay value ratio useful as a marker, wherein a combination of coherence values or a combination of phase delay values used in the division as a denominator comprises a sum of a set of coherence values or phase delay values, respectively.

In a further embodiment of the method, the sum of a set of coherence values or phase delay values is normalized by dividing with number of values within the set.

In one embodiment of the method, the combining the coherence value(s) or phase delay value(s) is or comprises dividing one coherence value by a second coherence value so as to obtain a coherence value ratio useful as a marker, or dividing one phase delay value by a second phase delay value so as to obtain a phase delay value ratio useful as a marker.

In another embodiment of the method, combining the coherence value(s) is or comprises dividing one coherence value by a second coherence value so as to obtain a coherence value ratio useful as a marker.

In one embodiment of the invention, a neuromarker for diagnosing PTSD based on single marker or single coherence value ratio for the sleep stage corresponding to awake period with lights off and before falling asleep (W) from single frequency or frequency band includes any of: Coh. F3-F4 (@ 8.6 Hz)/Coh. C3-F3 (@ 6.8 Hz), and Coh. F3-F4 (8.6-9.6 Hz)/Coh. C3-F3 (@ 6.8-7.8 Hz).

In a further embodiment of the method, the designated threshold of 0.812 and above signifies PTSD or likelihood of PTSD in the subject for the Coh. F3-F4 (@ 8.6 Hz)/Coh. C3-F3 (@ 6.8 Hz) neuromarker.

In another further embodiment of the method, the designated threshold of 0.8565 and above signifies PTSD or likelihood of PTSD in the subject for the Coh. F3-F4 (8.6-9.6 Hz)/Coh. C3-F3 (@ 6.8-7.8 Hz) neuromarker of (b).

In yet a further embodiment, a neuromarker for diagnosing PTSD based on single marker or single coherence value ratio for the particular sleep stage corresponding to stage II sleep (S2) from single frequency or frequency band includes any of Coh. C3-C4 (@11.2 Hz)/Coh. O2-C4 (@ 16.6 Hz); Coh. C3-C4 (@ 0.6 Hz)/Coh. C3-F4 (@ 2.6 Hz); Coh. C3-F3 (@ 11.8 Hz)/Coh. O2-F3 (@ 6.8 Hz); Coh. C4-F4 (@ 41.4 Hz)/Coh. C3-F4 (@ 2.6 Hz); and Coh. C3-F3 (19.4-20.4 Hz)/Coh. O2-C3 (0.6-1.6 Hz)

In a further embodiment of the method, the designated threshold of 1.4539 and above signifies PTSD or likelihood of PTSD in the subject for the Coh. C3-C4 (@ 11.2 Hz)/Coh. O2-C4 (@ 16.6 Hz) neuromarker. In another further embodiment of the method, the designated threshold of 0.834 and below signifies PTSD or likelihood of PTSD in the subject for the Coh. C3-C4 (@ 0.6 Hz)/Coh. C3-F4 (@ 2.6 Hz) neuromarker of (b). In another further embodiment of the method, the designated threshold of 1.1263 and below signifies PTSD or likelihood of PTSD in the subject for the Coh. C3-F3 (@ 11.8 Hz)/Coh. O2-F3 (@ 6.8 Hz) neuromarker of (c). In another further embodiment of the method, the designated threshold of 1.0818 and below signifies PTSD or likelihood of PTSD in the subject for Coh. C4-F4 (@ 41.4 Hz)/Coh. C3-F4 (@2.6 Hz) neuromarker of (d). In another further embodiment of the method, the designated threshold of 1.814 and below signifies PTSD or likelihood of PTSD in the subject for Coh. C3-F3 (19.4-20.4 Hz)/Coh. O2-C3 (0.6-1.6 Hz) neuromarker of (e).

In one embodiment of the method, the designated threshold is determined from comparison of set of values from control subjects and a second set of values from PTSD subjects and its value is set such as to permit discrimination between control and PTSD subjects.

In a further embodiment of the method, the comparison comprises statistics and statistical analysis.

In an additional embodiment of the method, the comparison comprises neuromarker values of PTSD subjects to have a mean, medium and/or mode value different from control subjects with a p-value less than 0.01. In another embodiment of the method, the comparison comprises neuromarker values of PTSD subjects to have a mean, medium and/or mode value different from control subjects with a p-value less than 0.001. In one embodiment of the method, F statistics from an ANOVA test or regression analysis has a p-value of less than 0.01. In another embodiment of the method, F statistics from an ANOVA test or regression analysis has a p-value of less than 0.001. In another embodiment of the method, F statistics from an ANOVA test or regression analysis has a p-value of less than 0.0001. In another embodiment of the method, F statistics from an ANOVA test or regression analysis has a p-value of less than 0.00001. In yet another embodiment of the method, F statistics from an ANOVA test or regression analysis has a p-value of less than 0.000001.

In one embodiment of the method, the subject with coherence value(s), phase delay value(s) and/or a combination thereof above a designated threshold is considered to have PTSD for mean, median and/or mode of coherence values, phase delay values and/or a combination thereof of control subjects below PTSD subjects, or alternatively below a designated threshold is considered to have PTSD for mean, median and/or mode of coherence values, phase delay values of control subjects above PTSD subjects.

In another embodiment of the method, the neuromarker value above a designated threshold is considered to have PTSD for mean, median and/or mode of neuromarker values of control subjects below PTSD subjects, or alternatively below a designated threshold is considered to have PTSD for mean, median and/or mode of neuromarker values of control subjects above PTSD subjects.

In yet a further embodiment, the coherence values for the sleep stage corresponding to stage II sleep (S2) and for single frequency of brain wave patterns recorded at two scalp locations includes any of Coh. O1-O2 (@ 6.2 Hz); Coh. C3-C4 (@ 7.0 Hz); Coh. C3-C4 (@ 11.2 Hz); Coh. O2-C4 (@ 16.6 Hz); Coh. C3-C4 (@ 11.0 Hz); Coh. O2-C3 (@16.4 Hz); Coh. C3-C4 (@0.6 Hz); Coh. C3-F4 (@ 2.6 Hz); Coh. O1-C3 (@6.6 Hz); Coh. O2-C3 (@ 0.6 Hz); Coh. O1-C3 (@0.6 Hz); Coh. O1-F3 (@ 6.8 Hz); Coh. C3-F3 (@ 11.8 Hz); Coh. O2-F3 (@ 6.8 Hz); Coh. C4-F4 (@ 41.4 Hz); Coh. C3-F4 (@2.6 Hz); Coh. C4-F4 (@ 13.4 Hz); Coh. O1-F3 (@ 15.8 Hz); Coh. O1-C4 (@7.0 Hz); and Coh. C3-F4 (@ 3.2 Hz); or a combination thereof.

In yet an additional embodiment, the coherence values for the sleep stage corresponding to stage II sleep (S2) and for frequency band from brain wave patterns recorded at two scalp locations including any of Coh. O1-O2 (6.2-7.2 Hz); Coh. C3-C4 (7.0-8.0 Hz); Coh. C3-C4 (7.0-8.0 Hz); Coh. F3-F4 (8.6-9.6 Hz); Coh. C3-C4 (11.2-12.20 Hz); Coh. O2-C4 (16.6-17.6 Hz); Coh. C3-C4 (11.0-12.0 Hz); Coh. O2-C3 (16.4-17.4 Hz); Coh. C3-F3 (19.4-20.4 Hz); Coh. O2-C3 (0.6-1.6 Hz); Coh. C4-F4 (41.4-42.4 Hz); and Coh. C3-F4 (2.6-3.6 Hz); or a combination thereof.

Also, in yet a further embodiment, the coherence value ratio useful as a marker for the sleep stage corresponding to stage II sleep (S2) for coherence values obtained from single frequency includes any of Coh. O1-O2 (@ 6.2 Hz)/Coh. C3-C4 (@ 7.0 Hz); Coh. C3-C4 (@ 11.2 Hz)/Coh. O2-C4 (@ 16.6 Hz); Coh. C3-C4 (@ 11.0 Hz)/Coh. O2-C3 (@ 16.4 Hz); Coh. C3-C4 (@ 0.6 Hz)/Coh. C3-F4 (@ 2.6 Hz); Coh. O1-C3 (@6.6 Hz)/Coh. O2-C3 (@ 0.6 Hz); Coh. O1-C3 (@0.6 Hz)/Coh. O1-F3 (@ 6.8 Hz); Coh. C3-F3 (@ 11.8 Hz)/Coh. O2-F3 (@ 6.8 Hz); Coh. C4-F4 (@41.4 Hz)/Coh. C3-F4 (@2.6 Hz); Coh. C4-F4 (@13.4 Hz)/Coh. O1-F3 (@ 15.8 Hz); and Coh. O1-C4 (@ 7.0 Hz)/Coh. C3-F4 (@ 3.2 Hz); or a combination thereof.

Further, in yet another embodiment, the coherence value ratio useful as a marker for the sleep stage corresponding to stage II sleep (S2) for coherence values obtained from frequency band includes any of Coh. O1-O2 (6.2-7.2 Hz)/Coh. C3-C4 (7.0-8.0 Hz); Coh. C3-C4 (7.0-8.0 Hz)/Coh. F3-F4 (8.6-9.6 Hz); Coh. C3-C4 (11.2-12.20 Hz)/Coh. O2-C4 (16.6-17.6 Hz); Coh. C3-C4 (11.0-12.0 Hz)/Coh. O2-C3 (16.4-17.4 Hz); Coh. C3-F3 (19.4-20.4 Hz)/Coh. O2-C3 (0.6-1.6 Hz); and Coh. C4-F4 (41.4-42.4 Hz)/Coh. C3-F4 (2.6-3.6 Hz); or a combination thereof. Merely by way of example, the width of the frequency band may be 1 Hz.

In an embodiment of the invention, the combination for the sleep stage corresponding to awake period with lights off and before falling asleep (W) at single frequency is or comprises multiple linear regression of two or more markers includes any of Coh. O1-O2 (@ 0.6 Hz)/Coh. O1-F4 (@ 2.4 Hz); Coh. C3-C4 (@ 23.8 Hz)/Coh. O2-C3 (@ 37.0 Hz); Coh. F3-F4 (@ 8.6 Hz)/Coh. C3-F3 (@ 6.8 Hz); Coh. F3-F4 (@ 7.4 Hz)/Coh. C3-F4 (@ 7.6 Hz); Coh. F3-F4 (@ 16.8 Hz)/Coh. O1-F4 (@ 7.4 Hz); and Coh. O1-F4 (@4.2 Hz)/Coh. O2-F3 (@ 13.0 Hz).

In yet another embodiment, the neuromarker comprises all six markers as provided hereinabove.

In one embodiment, the value of the neuromarker for diagnosing PTSD, designated PTSD_Diag_Wake_1 neuromarker, is the sum of −0.11×[Coh. O1-O2 (@ 0.6 Hz)/Coh. O1-F4 (@ 2.4 Hz)]; −0.27×[Coh. C3-C4 (@ 23.8 Hz)/Coh. O2-C3 (@ 37.0 Hz)]; 0.72×[Coh. F3-F4 (@ 8.6 Hz)/Coh. C3-F3 (@ 6.8 Hz)]; −0.07×[Coh. F3-F4 (@ 7.4 Hz)/Coh. C3-F4 (@ 7.6 Hz)]; −0.11×[Coh. F3-F4 (@ 16.8 Hz)/Coh. O1-F4 (@ 7.4 Hz)]; −0.31×[Coh. O1-F4 (@ 4.2 Hz)/Coh. O2-F3 (@ 13.0 Hz)]; and 1.92.

In yet another embodiment, the values of PTSD_Diag_Wake_1 neuromarker obtained for a control population and a PTSD population have mean values with standard deviations of 1.11±0.18 and 1.90±0.23, respectively.

In yet a further embodiment, the values of PTSD_Diag_Wake_1 neuromarker obtained for a control population and a PTSD population have median values of 1.12 and 1.88, respectively. For example, the threshold value of 1.5 for PTSD_Diag_Wake_1 neuromarker and above may indicate the presence of PTSD in a subject.

Additionally, in one embodiment, the combination for the sleep stage corresponding to awake period with lights off and before falling asleep (W) for coherence value ratios obtained from frequency band is or comprises multiple linear regression of two markers such as Coh. F3-F4 (8.6-9.6 Hz)/Coh. C3-F3 (@ 6.8-7.8 Hz); and Coh. F3-F4 (@7.6-8.6 Hz)/Coh. O2-F3 (@ 7.4-8.4 Hz).

In yet an additional embodiment, the value of the neuromarker for diagnosing PTSD, designated PTSD_Diag_Wake_2 neuromarker, may be the sum of 0.92×[Coh. F3-F4 (8.6-9.6 Hz)/Coh. C3-F3 (@ 6.8-7.8 Hz)]; −0.27×[Coh. F3-F4 (@ 7.6-8.6 Hz)/Coh. O2-F3 (@ 7.4-8.4 Hz)]; and 1.16.

In yet another embodiment, the values of PTSD_Diag_Wake_2 neuromarker obtained for a control population and a PTSD population have mean values with standard deviations of 1.08±0.12 and 1.92±0.24, respectively.

In an additional embodiment, the values of PTSD_Diag_Wake_2 neuromarker obtained for a control population and a PTSD population have median values of 1.07 and 1.83, respectively. For example, the threshold value of 1.5 for PTSD_Diag_Wake_2 neuromarker value of 1.5 and above may indicate presence of PTSD in a subject.

In one embodiment, the combination for the sleep stage corresponding to stage II sleep (S2) at single frequency may be or may comprise a multiple linear regression of two or more markers include any of Coh. O1-O2 (@ 6.2 Hz)/Coh. C3-C4 (@ 7.0 Hz); Coh. C3-C4 (@ 11.2 Hz)/Coh. O2-C4 (@ 16.6 Hz); Coh. C3-C4 (@ 11.0 Hz)/Coh. O2-C3 (@ 16.4 Hz); Coh. C3-C4 (@ 0.6 Hz)/Coh. C3-F4 (@ 2.6 Hz); Coh. O1-C3 (@6.6 Hz)/Coh. O2-C3 (@ 0.6 Hz); Coh. O1-C3 (@0.6 Hz)/Coh. O1-F3 (@6.8 Hz); Coh. C3-F3 (@11.8 Hz)/Coh. O2-F3 (@ 6.8 Hz); Coh. C4-F4 (@41.4 Hz)/Coh. C3-F4 (@ 2.6 Hz); Coh. C4-F4 (@ 13.4 Hz)/Coh. O1-F3 (@ 15.8 Hz); and Coh. O1-C4 (@ 7.0 Hz)/Coh. C3-F4 (@ 3.2 Hz). In yet another embodiment, the neuromarker comprises all ten markers as provided herein above.

In yet a further embodiment, the value of neuromarker for diagnosing PTSD, designated PTSD_Diag_Stage2_1 neuromarker, may be the sum of −0.16×[Coh. O1-O2 (@ 6.2 Hz)/Coh. C3-C4 (@ 7.0 Hz)]; 0.17×[Coh. C3-C4 (@ 11.2 Hz)/Coh. O2-C4 (@ 16.6 Hz)]; −0.17×[Coh. C3-C4 (@ 11.0 Hz)/Coh. O2-C3 (@ 16.4 Hz)]; −0.07×[Coh. C3-C4 (@ 0.6 Hz)/Coh. C3-F4 (@ 2.6 Hz)]; −0.09×[Coh. O1-C3 (@ 6.6 Hz)/Coh. O2-C3 (@ 0.6 Hz)]; −0.08×[Coh. O1-C3 (@ 0.6 Hz)/Coh. O1-F3 (@ 6.8 Hz)]; −0.05×[Coh. C3-F3 (@ 11.8 Hz)/Coh. O2-F3 (@ 6.8 Hz)]; −0.09×[Coh. C4-F4 (@ 41.4 Hz)/Coh. C3-F4 (@ 2.6 Hz)]; −0.10×[Coh. C4-F4 (@13.4 Hz)/Coh. O1-F3 (@ 15.8 Hz)]; −0.07×[Coh. O1-C4 (@ 7.0 Hz)/Coh. C3-F4 (@ 3.2 Hz]; and 2.71.

In an embodiment of the invention, the values of PTSD_Diag_Stage2_1l neuromarker may be obtained for a control population and a PTSD population may have mean values with standard deviations of 1.02+0.13 and 1.98+0.05, respectively.

In an additional embodiment, the values of PTSD_Diag_Stage2_1 neuromarker may be obtained for a control population and a PTSD population have median values of 1.03 and 1.98, respectively.

In yet another embodiment, the threshold value of 1.75 for PTSD_Diag_Stage2_1l neuromarker and above indicates presence of PTSD in a subject.

In a further embodiment, the combination for the sleep stage corresponding to stage II sleep (S2) for frequency band is or comprises multiple linear regression of two or more markers includes any of Coh. O1-O2 (6.2-7.2 Hz)/Coh. C3-C4 (7.0-8.0 Hz); Coh. C3-C4 (7.0-8.0 Hz)/Coh. F3-F4 (8.6-9.6 Hz); Coh. C3-C4 (11.2-12.20 Hz)/Coh. O2-C4 (16.6-17.6 Hz); Coh. C3-C4 (11.0-12.0 Hz)/Coh. O2-C3 (16.4-17.4 Hz); Coh. C3-F3 (19.4-20.4 Hz)/Coh. O2-C3 (0.6-1.6 Hz); and Coh. C4-F4 (41.4-42.4 Hz)/Coh. C3-F4 (2.6-3.6 Hz). Additionally, in one embodiment, the neuromarker comprises all six markers as provided hereinabove.

Also, in another embodiment, the value of the neuromarker for diagnosing PTSD, designated PTSD_Diag_Stage2_2 neuromarker, is the sum of −0.17×[Coh. O1-O2 (6.2-7.2 Hz)/Coh. C3-C4 (7.0-8.0 Hz)]; −0.02×[Coh. C3-C4 (7.0-8.0 Hz)/Coh. F3-F4 (8.6-9.6 Hz)]; 0.2305×[Coh. C3-C4 (11.2-12.20 Hz)/Coh. O2-C4 (16.6-17.6 Hz)]; −0.25×[Coh. C3-C4 (11.0-12.0 Hz)/Coh. O2-C3 (16.4-17.4 Hz)]; −0.09×[Coh. C3-F3 (19.4-20.4 Hz)/Coh. O2-C3 (0.6-1.6 Hz)]; −0.12×[Coh. C4-F4 (41.4-42.4 Hz)/Coh. C3-F4 (2.6-3.6 Hz)]; and 2.40.

Further, in another embodiment, the values of PTSD_DiagStage2_2 neuromarker obtained for a control population and a PTSD population have mean values with standard deviations of 1.04±0.19 and 1.96±0.03, respectively. For example, the invention encompasses, in one embodiment the values of PTSD_Diag_Stage2_2 neuromarker obtained for a control population and a PTSD population have median values of 1.02 and 1.96, respectively.

In one embodiment of the invention, the designated threshold value of 1.8 for PTSD_Diag_Stage2_2 neuromarker and above indicates presence of PTSD in a subject.

Merely by way of example, the combination of markers using multiple linear regression comprises multiple linear regression of markers from one sleep stage. Further, for example, the combination may comprise or further comprise an arithmetic operation, wherein the arithmetic operation is selected from the group consisting of addition, subtraction, division and multiplication. In one embodiment, the mathematical combination comprises multiplication of value of multiple linear regression of markers from one sleep stage with value of multiple linear regression of markers from a different sleep stage. In another embodiment, the mathematical combination is multiplication of value of multiple linear regression of markers from one sleep stage with value of multiple linear regression of markers from a different sleep stage.

In one embodiment of the invention, the neuromarker is a combination of markers from a sleep stage corresponding to awake period with lights off and before falling asleep (W) and a marker or combination of markers from a sleep stage corresponding to stage II sleep (S2). In another embodiment, the neuromarker from combination of two or more markers from two or more sleep stages is a combination of neuromarker from sleep stage corresponding to awake period with lights off and before falling asleep (W) and a neuromarker from a sleep stage corresponding to stage II sleep (S2). In yet a further embodiment, the neuromarker for diagnosing PTSD from sleep stage corresponding to awake period with lights off and before falling asleep (W) includes any of a PTSD_Diag_Wake_1 neuromarker as described herein and a PTSD_Diag_Wake_2 neuromarker as described herein. Further, in another embodiment, the neuromarker for diagnosing PTSD for sleep stage corresponding to stage II sleep (S2) is selected from the group consisting of PTSD_Diag_Stage2_1 neuromarker as described herein and PTSD_Diag_Stage2_2 neuromarker as described herein. Further still, in yet another embodiment, the neuromarker for diagnosing PTSD from the combination of two or more markers from two or more sleep stages is a combination of a PTSD_Diag_Wake_1 neuromarker as described herein and a PTSD_Diag Stage2_1_neuromarker as described herein.

In yet a further embodiment, the neuromarker for diagnosing PTSD, designated PTSD_Diag_S2×W_1_neuromarker, is a product of PTSD_Diag_Wake_1_neuromarker and PTSD_Diag_Stage2_1_neuromarker, wherein PTSD_Diag_Wake_1_neuromarker includes any of [−0.11×[Coh. O1-O2 (@ 0.6 Hz)/Coh. O1-F4 (@ 2.4 Hz)]−0.27×[Coh. C3-C4 (@ 23.8 Hz)/Coh. O2-C3 (@ 37.0 Hz)]+0.72×[Coh. F3-F4 (@ 8.6 Hz)/Coh. C3-F3 (@ 6.8 Hz)]−0.07×[Coh. F3-F4 (@ 7.4 Hz)/Coh. C3-F4 (@ 7.6 Hz)]−0.11×[Coh. F3-F4 (@ 16.8 Hz)/Coh. O1-F4 (@ 7.4 Hz)]−0.31×[Coh. O1-F4 (@ 4.2 Hz)/Coh. O2-F3 (@ 13.0 Hz)]+1.92], and wherein PTSD_Diag_Stage2_1 neuromarker is: [−0.16×[Coh. O1-O2 (@ 6.2 Hz)/Coh. C3-C4 (@ 7.0 Hz)]+0.17×[Coh. C3-C4 (@ 11.2 Hz)/Coh. O2-C4 (@ 16.6 Hz)]−0.17×[Coh. C3-C4 (@11.0 Hz)/Coh. O2-C3 (@ 16.4 Hz)]−0.07×[Coh. C3-C4 (@ 0.6 Hz)/Coh. C3-F4 (@ 2.6 Hz)]−0.09×[Coh. O1-C3 (@ 6.6 Hz)/Coh. O2-C3 (@ 0.6 Hz)]−0.08×[Coh. O1-C3 (@ 0.6 Hz)/Coh. O1-F3 (@ 6.8 Hz)]−0.05×[Coh. C3-F3 (@11.8 Hz)/Coh. O2-F3 (@ 6.8 Hz)]−0.09×[Coh. C4-F4 (@ 41.4 Hz)/Coh. C3-F4 (@ 2.6 Hz)]−0.10×[Coh. C4-F4 (@ 13.4 Hz)/Coh. O1-F3 (@ 15.8 Hz)]−0.07×[Coh. O1-C4 (@ 7.0 Hz)/Coh. C3-F4 (@ 3.2 Hz]+2.71]. Merely by way of example, the values of designated PTSD_Diag_S2×W_1 neuromarker for a control population and a PTSD population may have mean values with standard deviations of 1.13±0.26 and 3.75±0.45, respectively. For example, the values of designated PTSD_Diag_S2×W_1 neuromarker for a control population and a PTSD population may have median values of 1.16 and 3.69, respectively. In one embodiment of the invention, the designated threshold value of 2.3 for PTSD_Diag_S2×W_1 and above may indicate presence of PTSD in a subject.

Also, in a further embodiment, the neuromarker for diagnosing PTSD from combination of two or more markers from two or more sleep stages is a combination of a PTSD_Diag_Wake_2 neuromarker as described herein and a PTSD_Diag_Stage2_2 neuromarkers described herein. For example, in one embodiment, the neuromarker for diagnosing PTSD, designated PTSD_DiagS2×W_2 neuromarker, is a product of PTSD_Diag_Wake_2 neuromarker and PTSD_Diag_Stage2_2 neuromarker, wherein PTSD_Diag_Wake_2 neuromarker is [0.92×[Coh. F3-F4 (8.6-9.6 Hz)/Coh. C3-F3 (@ 6.8-7.8 Hz)]—0.27×[Coh. F3-F4 (@ 7.6-8.6 Hz)/Coh. O2-F3 (@ 7.4-8.4 Hz)]+1.16], and wherein PTSD_Diag_Stage2_2 neuromarker is: [−0.17×[Coh. O1-O2 (6.2-7.2 Hz)/Coh. C3-C4 (7.0-8.0 Hz)]—0.02×[Coh. C3-C4 (7.0-8.0 Hz)/Coh. F3-F4 (8.6-9.6 Hz)]+0.2305×[Coh. C3-C4 (11.2-12.20 Hz)/Coh. O2-C4 (16.6-17.6 Hz)]—0.25×[Coh. C3-C4 (11.0-12.0 Hz)/Coh. O2-C3 (16.4-17.4 Hz)]—0.09×[Coh. C3-F3 (19.4-20.4 Hz)/Coh. O2-C3 (0.6-1.6 Hz)]—0.12×[Coh. C4-F4 (41.4-42.4 Hz)/Coh. C3-F4 (2.6-3.6 Hz)]+2.40].

For example, the values of designated PTSD_Diag_S2×W_2 neuromarker for a control population and a PTSD population may have mean values with standard deviations of 1.12+0.22 and 3.76+0.48, respectively. In one embodiment, the values of designated PTSD_Diag_S2×W_2 neuromarker for a control population and a PTSD population may have median values of 1.12 and 3.56, respectively. In another embodiment, the designated threshold of 2.5 for PTSD_Diag_S2×W_2 neuromarker and above may indicate presence of PTSD in a subject.

The present invention in one aspect provides a method for determining severity of PTSD symptom in a subject. In one embodiment, the method comprises a) obtaining two or more brain wave patterns from at least two locations selected on a head of a subject; b) segmentingthe brain wave patterns by sleep stage; c) segmenting the brain wave patterns for one sleep stage so segmented from step b into defined time intervals so as to permit auto and cross spectral analysis and/or coherence analysis; d) calculating coherence value(s) and/or phase delay value(s) from two brain wave segments of step c for a single frequency or a frequency band at a sleep stage; and d) repeating step (d) to obtain more coherence values and/or phase delay values for the same sleep stage, at (i) other single frequency or frequency band obtained from the same two locations, and/or (ii) the same or other single frequency or frequency band obtained from two different locations or two locations in which one location is shared in common in step (d). Optionally, the method further comprises e) performing steps (d) and (e) for a different sleep stage or multiple sleep stages. The method further comprises f) combining coherence value(s) or phase delay value(s) so as to be a marker or a combination of markers which may serve as a neuromarker for PTSD symptom severity or may be combined with other markers to produce a neuromarker for PTSD symptom severity; g) combining coherence value(s) or phase delay value(s) so as to be a marker or a combination of markers; h) selecting a neuromarker from the markers or combination of markers of step (g), wherein the neuromarker is defined as: (i) a single coherence value ratio or phase delay value ratio; (ii) combination of two or more markers of step (g) for a sleep stage; and/or (iii) combination of two or more markers of step (g) from two or more sleep stages; wherein the neuromarker correlates with severity of PTSD symptom; i) determining value of the neuromarker so as to determine the severity of PTSD, wherein the higher value or its absolute value signifies greater severity of the PTSD symptom; thereby, determining severity of PTSD symptom in a subject.

In a preferred embodiment of the invention, PTSD symptom severity may be assessed by giving a score from 0 to 4 for each of the 20 items associated with the 4 PTSD symptom clusters described herein (Weathers, Frank W., et. al (2014). PTSD Checklist for DSM-5 (PCL-5)).

In one embodiment of the method, brain wave patterns are obtained in a sleep study comprising EEG. A sleep study is also referred to herein as Polysomnogram. It is a procedure where multiple biological functions during sleep are recorded and analyzed for determination of sleep structure (e.g., % time spent in various stages of sleep) and abnormalities (such as occurrence of apneic events). The signals that are recorded during a sleep study include, but are not limited to, brain wave activity, eye movement, muscle tone, heart rhythm and breathing via electrodes and monitors placed on the head, chest and legs. For further discussion of sleep stages and sleep analysis, see Berry R B, Brooks R, Gamaldo C E, Harding S M, Marcus C L and Vaughn B V for the American Academy of Sleep Medicine. The AASM Manual for the Scoring of Sleep and Associated Events: Rules, Terminology and Technical Specifications, Version 2.2. aasm.org.

In another embodiment of the method, the locations selected on a head of the subject include any of Fp1, Fp2, F3, F4, F7, F8, Fz, A1, A2, C3, C4, Cz, T3, T4, T5, T6, P3, P4, Pz, O1 and O2 of International 10-20 system. In an additional embodiment of the method, the locations include any of F3, F4, C3, C4, O1 and O2. In another embodiment of the method, the brain wave patterns are recorded simultaneously.

In another embodiment of the method, brain wave patterns are segmented into sleep stages, and examples of the sleep stages include, but are not limited to, awake period with lights off and before falling asleep (W), stage I sleep, stage II sleep (S2), delta-wave or stable III sleep and rapid-eye-movement (REM) sleep.

In another embodiment of the method, the sleep stage is either awake period with lights off and before falling asleep (W) or stage II sleep (S2). In another embodiment of the method, the sleep stage is awake period with lights off and before falling asleep (W). In another embodiment of the method, the sleep stage is stage II sleep (S2).

In another embodiment of the method, the defined time interval is greater than 2 seconds and less than 30 seconds. In another embodiment of the method, the defined time interval is about 5 seconds.

In another embodiment of the method, the single frequency is selected from any frequency between 0 Hz and 52 Hz. In another embodiment of the method, the single frequency includes any of about 0.2, 0.4, 0.6, 0.8, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 12.2, 12.4, 12.6, 12.8, 13.0, 13.2, 13.4, 13.6, 13.8, 14.0, 14.2, 14.4, 14.6, 14.8, 15.0, 15.2, 15.4, 15.6, 15.8, 16.0, 16.2, 16.4, 16.6, 16.8, 17.0, 17.2, 17.4, 17.6, 17.8, 18.0, 18.2, 18.4, 18.6, 18.8, 19.0, 19.2, 19.4, 19.6, 19.8, 20.0, 20.2, 20.4, 20.6, 20.8, 21.0, 21.2, 21.4, 21.6, 21.8, 22.0, 22.2, 22.4, 22.6, 22.8, 23.0, 23.2, 23.4, 23.6, 23.8, 24.0, 24.2, 24.4, 24.6, 24.8, 25.0, 25.2, 25.4, 25.6, 25.8, 26.0, 26.2, 26.4, 26.6, 26.8, 27.0, 27.2, 27.4, 27.6, 27.8, 28.0, 28.2, 28.4, 28.6, 28.8, 29.0, 29.2, 29.4, 29.6, 29.8, 30.0, 30.2, 30.4, 30.6, 30.8, 33.0, 33.2, 33.4, 33.6, 33.8, 32.0, 32.2, 32.4, 32.6, 32.8, 33.0, 33.2, 33.4, 33.6, 33.8, 34.0, 34.2, 34.4, 34.6, 34.8, 35.0, 35.2, 35.4, 35.6, 35.8, 36.0, 36.2, 36.4, 36.6, 36.8, 37.0, 37.2, 37.4, 37.6, 37.8, 38.0, 38.2, 38.4, 38.6, 38.8, 39.0, 39.2, 39.4, 39.6, 39.8, 40.0, 40.2, 40.4, 40.6, 40.8, 44.0, 44.2, 44.4, 44.6, 44.8, 42.0, 42.2, 42.4, 42.6, 42.8, 43.0, 43.2, 43.4, 43.6, 43.8, 44.0, 44.2, 44.4, 44.6, 44.8, 45.0, 45.2, 45.4, 45.6, 45.8, 46.0, 46.2, 46.4, 46.6, 46.8, 47.0, 47.2, 47.4, 47.6, 47.8, 48.0, 48.2, 48.4, 48.6, 48.8, 49.0, 49.2, 49.4, 49.6, 49.8, 50.0, 50.2, 50.4, 50.6, 50.8, 51.0, 51.2, 51.4, 51.6, 51.8 and 52.0 Hz. In accordance with the practice of the invention, the single frequency may vary by about ±0.1 Hz.

In an additional embodiment of the method, the single frequency includes any of 0.2, 0.6, 1.0, 2.8, 5.0, 6.4, 6.8, 7.6, 8.4, 10.0, 11.8, 12.8, 19.6, 19.8, 20.0, 20.4, 20.6, 37.8, 38.8, 39.6 and 45.6 Hz. In another embodiment of the method, the single frequency includes any of 0.2, 2.8, 6.8, 7.6, 8.4, 12.8, 19.6, 20.0, 20.4, 20.6, 38.8 and 45.6 Hz for the sleep stage corresponding to awake period with lights off and before falling asleep (W). Examples of the single frequency include, but are not limited to 0.6, 1.0, 5.0, 6.4, 10.0, 11.8, 19.6, 19.8, 37.8 and 39.6 Hz for the sleep stage corresponding to stage II sleep (S2). In some embodiments of the method, the single frequency may vary by any value within a 2 Hz window. In some embodiments of the method, the single frequency may vary by any value within a 0.2 Hz window. In an additional embodiment of the method, the single frequency may vary by any value within a 0.1 Hz window. Examples of the frequency band include, but are not limited to, any of 0.2, 0.6, 1.0, 2.8, 5.0, 6.4, 6.8, 7.6, 8.4, 10.0, 11.8, 12.8, 19.6, 19.8, 20.0, 20.4, 20.6, 37.8, 38.8, 39.6 and 45.6 Hz. In one embodiment of the method, examples of the frequency band include, but are not limited to, 0.2, 2.8, 6.8, 7.6, 8.4, 12.8, 19.6, 20.0, 20.4, 20.6, 38.8 and 45.6 Hz for the sleep stage corresponding to awake period with lights off and before falling asleep (W). In another embodiment of the method, examples of the frequency band include, but are not limited to, 0.6, 1.0, 5.0, 6.4, 10.0, 11.8, 19.6, 19.8, 37.8 and 39.6 Hz for the sleep stage corresponding to stage II sleep (S2). In some embodiments of the method, width of the frequency band is least a 0.1 Hz but less than 20 Hz. In some embodiments of the method, width of the frequency band is least a 0.2 Hz but less than 10 Hz. In some embodiments of the method, width of the frequency band includes any of 0.1 Hz, 0.2 Hz, 0.5 Hz, 1 Hz, 1.5 Hz, 2 Hz, 2.5 Hz, 3 Hz, 3.5 Hz, 4 Hz, 4.5 Hz, 5 Hz, 5.5 Hz, 6 Hz, 6.5 Hz, 7 Hz, 7.5 Hz, 8 Hz, 8.5 Hz, 9 Hz, 9.5 Hz and 10 Hz. In some embodiments of the method, width of the frequency band is 1 Hz. In another embodiment of the method, width of the frequency band is 1 Hz selected over the frequency between 0-52 Hz.

Examples of the frequency band include, but are not limited to, 0.2-1.2, 0.4-1.4, 0.6-1.6, 0.8-1.8, 1.0-2.0, 1.2-2.2, 2.4-3.4, 2.6-3.6, 2.8-3.8, 3.0-4.0, 3.2-4.2, 3.4-4.4, 3.6-4.6, 3.8-4.8, 4.0-5.0, 4.2-5.2, 4.4-5.4, 4.6-5.6, 4.8-5.8, 5.0-6.0, 5.2-6.2, 5.4-6.4, 5.6-6.6, 5.8-6.8, 6.0-7.0, 6.2-7.2, 6.4-7.4, 6.6-7.6, 6.8-7.8, 7.0-8.0, 7.2-8.2, 7.4-8.4, 7.6-8.6, 7.8-8.8, 8.0-9.0, 8.2-9.2, 8.4-9.4, 8.6-9.6, 8.8-9.8, 9.0-10.0, 9.2-10.2, 9.4-10.4, 9.6-10.6, 9.8-10.8, 10.0-11.0, 10.2-11.2, 10.4-11.4, 10.6-11.6, 10.8-11.8, 11.0-12.0, 11.2-12.2, 11.4-12.4, 11.6-12.6, 11.8-12.8, 12.0-13.0, 12.2-13.2, 12.4-13.4, 12.6-13.6, 12.8-13.8, 13.0-14.0, 13.2-14.2, 13.4-14.4, 13.6-14.6, 13.8-14.8, 14.0-15.0, 15.2-16.2, 15.4-16.4, 15.6-16.6, 15.8-16.8, 16.0-17.0, 16.2-17.2, 16.4-17.4, 16.6-17.6, 16.8-17.8, 17.0-18.0, 17.2-18.2, 17.4-18.4, 17.6-18.6, 17.8-18.8, 18.0-19.0, 18.2-19.2, 18.4-19.4, 18.6-19.6, 18.8-19.8, 19.0-20.0, 19.2-20.2, 19.4-20.4, 19.6-20.6, 19.8-20.8, 20.0-21.0, 20.2-21.2, 20.4-21.4, 20.6-21.6, 20.8-21.8, 21.0-22.0, 21.2-22.2, 21.4-22.4, 21.6-22.6, 21.8-22.8, 22.0-23.0, 22.2-23.2, 22.4-23.4, 22.6-23.6, 22.8-23.8, 23.0-24.0, 23.2-24.2, 23.4-24.4, 23.6-24.6, 23.8-24.8, 24.0-25.0, 24.2-25.2, 24.4-25.4, 24.6-25.6, 24.8-25.8, 25.0-26.0, 25.2-26.2, 25.4-26.4, 25.6-26.6, 25.8-26.8, 26.0-27.0, 26.2-27.2, 26.4-27.4, 26.6-27.6, 26.8-27.8, 27.0-28.0, 27.2-28.2, 27.4-28.4, 27.6-28.6, 27.8-28.8, 28.0-29.0, 28.2-29.2, 28.4-29.4, 28.6-29.6, 28.8-29.8, 29.0-30.0, 29.2-30.2, 29.4-30.4, 29.6-30.6, 29.8-30.8, 30.0-31.0, 30.2-31.2, 30.4-31.4, 30.6-31.6, 30.8-31.8, 31.0-32.0, 31.2-32.2, 31.4-32.4, 31.6-32.6, 31.8-32.8, 32.0-33.0, 32.2-33.2, 32.4-33.4, 32.6-33.6, 32.8-33.8, 33.0-34.0, 33.2-34.2, 33.4-34.4, 33.6-34.6, 33.8-34.8, 34.0-35.0, 34.2-35.2, 34.4-35.4, 34.6-35.6, 34.8-35.8, 35.0-36.0, 35.2-36.2, 35.4-36.4, 35.6-36.6, 35.8-36.8, 36.0-37.0, 36.2-37.2, 36.4-37.4, 36.6-37.6, 36.8-37.8, 37.0-38.0, 37.2-38.2, 37.4-38.4, 37.6-38.6, 37.8-38.8, 38.0-39.0, 38.2-39.2, 38.4-39.4, 38.6-39.6, 38.8-39.8, 39.0-40.0, 39.2-40.2, 39.4-40.4, 39.6-40.6, 39.8-40.8, 40.0-41.0, 40.2-41.2, 40.4-41.4, 40.6-41.6, 40.8-41.8, 41.0-42.0, 41.2-42.2, 41.4-42.4, 41.6-42.6, 41.8-42.8, 42.0-43.0, 42.2-43.2, 42.4-43.4, 42.6-43.6, 42.8-43.8, 43.0-44.0, 43.2-44.2, 43.4-44.4, 43.6-44.6, 43.8-44.8, 44.0-45.0, 44.2-45.2, 44.4-45.4, 44.6-45.6, 44.8-45.8, 45.0-46.0, 46.2-47.2, 46.4-47.4, 46.6-47.6, 46.8-47.8, 47.0-48.0, 47.2-48.2, 47.4-48.4, 47.6-48.6, 47.8-48.8, 48.0-49.0, 48.2-49.2, 48.4-49.4, 48.6-49.6, 48.8-49.8, 49.0-50.0, 49.2-50.2, 49.4-50.4, 49.6-50.6, 49.8-50.8, 50.0-51.0, 50.2-51.2, 50.4-51.4, 50.6-51.6, 50.8-51.8 and 51.0-52.0 Hz.

In another embodiment of the method, the frequency band may start anywhere within the frequency bands as provided above in the examples, but end outside of the examples above so long as a 1 Hz band width is maintained.

In another embodiment of the method, examples of the frequency band include, but are not limited to, 0.2-1.2, 0.6-1.6, 6.8-7.8, 7.4-8.4, 7.6-8.6, 8.4-9.4, 11.8-12.8, 12.6-13.6, 19.8-20.8, 20-21, 20.4-21.4, 29.4-30.4, 30.6-31.6, 37.8-38.8, 39.6-40.6, 40-41, 45.6-46.6, 48.2-49.2 and 49.8-50.8 Hz.

In another embodiment of the method, examples of the frequency band include, but are not limited to, 0.6-1.6, 6.8-7.8, 7.4-8.4, 7.6-8.6, 8.4-9.4, 12.6-13.6, 19.8-20.8, 20-21, 20.4-21.4, 40-41, 45.6-46.6, 49.8-50.8 Hz for the sleep stage corresponding to awake period with lights off and before falling asleep (W).

In another embodiment of the method, examples of the frequency band include, but are not limited to, 0.2-1.2, 11.8-12.8, 19.8-20.8, 29.4-30.4, 30.6-31.6, 37.8-38.8, 39.6-40.6 and 48.2-49.2 Hz for the sleep stage corresponding to stage II sleep (S2). In one embodiment of the method, the frequency band may start anywhere within 0.6-1.6, 2.6-3.6, 6.2-7.2, 6.8-7.8, 7.0-8.0, 7.4-8.4, 7.6-8.6, 8.6-9.6, 11.0-12.0, 11.2-12.2, 16.4-17.4, 16.6-17.6, 19.4-20.4 and 41.4-42.4 Hz., but end outside of this frequency band range to maintain a 1 Hz frequency band.

In one embodiment of the method, examples of the two scalp locations include, but are not limited to, O1-O2, C3-C4, F3-F4, O1-C3, O2-C4, C3-F3, C4-F4, O1-C4, O2-C3, C3-F4, C4-F3, O1-F3, O1-F4, O2-F3 and O2-F4. In another embodiment of the method, examples of the two scalp locations include, but are not limited to, O1-O2, C3-C4, F3-F4, O1-C3, C3-F3, O2-C3, C3-F4, C4-F3, O1-F3, O1-F4, O2-F3; and O2-F4. In another embodiment of the method, examples of the two scalp locations for the sleep stage corresponding to awake period with lights off and before falling asleep (W) include, but are not limited to, O1-O2, C3-C4, F3-F4, O1-C3, O2-C3, C3-F4, O1-F3, O2-F3 and O2-F4. In another embodiment of the method, examples of the two scalp locations for the sleep stage corresponding to stage II sleep (S2) include, but are not limited to, C3-F3, O2-C3, C3-F4, C4-F3, O1-F3, O1-F4; and O2-F3.

In one embodiment of the method, examples of the coherence values for the sleep stage corresponding to awake period with lights off and before falling asleep (W) and for single frequency of brain wave patterns recorded at two scalp locations include, but are not limited to, Coh. O1-C3 (@ 8.4 Hz), Coh. O2-F3 (@ 2.8 Hz), Coh. C4-F3 (@ 19.6 Hz), Coh. O2-F4 (@ 12.8 Hz), Coh. F3-F4 (@ 20.4 Hz), Coh. C3-F4 (@ 7.6 Hz), Coh. O1-C4 (@ 20 Hz), Coh. C3-F4 (@ 6.8 Hz), Coh. C3-F3 (@ 0.2 Hz), Coh. O1-F3 (@6.8 Hz), Coh. O1-C4 (@ 20.0 Hz), Coh. O1-F3 (@ 45.6 Hz), Coh. C3-C4 (@ 20.6 Hz), Coh. C3-F4 (@ 38.8 Hz), Coh. C3-C4 (@ 20.0 Hz); and Coh. O2-C3 (@ 6.8 Hz); and a combination thereof.

In one embodiment of the method, the combining the coherence value(s) or phase delay value(s) is a mathematical operation performed on two or more coherence value(s) or phase delay value(s), wherein the mathematical operation is selected from the group consisting of addition, subtraction, multiplication, division, factorial, sigma, n-th root, exponential, logarithm, mean, median, mode, standard deviation, coefficient of variation, geometric sequence, arithmetic sequence, normalization, binary, averaging, ratiometric, trigonometric function, linear function, exponential function, logarithmic function and function with input of coherence value or phase delay value as a dependent variable, regression function, linear regression, multiple linear regression, logistic regression, polynomial regression, nonlinear regression, nonparametric function and semiparametric function, and a combination thereof.

In a further embodiment of the method, the combining the coherence value(s) or phase delay value(s) is or comprises dividing one coherence value by a second coherence value or a combination of coherence values so as to obtain a coherence value ratio useful as a marker, dividing one phase delay value by a second phase delay value or a combination of phase delay values so as to obtain a phase delay value ratio useful as a marker, wherein a combination of coherence values or a combination of phase delay values used in the division as a denominator is obtained by performing a mathematical operation selected from the group consisting of addition, subtraction, multiplication, division, factorial, sigma, n-th root, exponential, logarithm, mean, median, mode, standard deviation, coefficient of variation, geometric sequence, arithmetic sequence, normalization, binary, averaging, ratiometric, trigonometric function, linear function, exponential function, logarithmic function, function with input of coherence value or phase delay value as a dependent variable, regression function, linear regression, multiple linear regression, logistic regression, polynomial regression, nonlinear regression, nonparametric function and semiparametric function, and a combination thereof, for a set of coherence values or phase delay values, respectively.

In another embodiment of the method, the combining the coherence value(s) or phase delay value(s) is or comprises dividing one coherence value by a second coherence value or a combination of coherence values so as to obtain a coherence value ratio useful as a marker, dividing one phase delay value by a second phase delay value or a combination of phase delay values so as to obtain a phase delay value ratio useful as a marker, wherein a combination of coherence values or a combination of phase delay values used in the division as a denominator comprises a sum of a set of coherence values or phase delay values, respectively.

In another embodiment of the method, the sum of a set of coherence values or phase delay values is normalized by dividing with number of values within the set.

In yet another embodiment of the method, the combining the coherence value(s) or phase delay value(s) is or comprises dividing one coherence value by a second coherence value so as to obtain a coherence value ratio useful as a marker, or dividing one phase delay value by a second phase delay value so as to obtain a phase delay value ratio useful as a marker.

In an embodiment of the method, the combining the coherence value(s) is or comprises dividing one coherence value by a second coherence value so as to obtain a coherence value ratio useful as a marker.

In another embodiment of the method, examples of the coherence value ratio useful as a marker for the sleep stage corresponding to awake period with lights off and before falling asleep (W) include, but are not limited to, Coh. O1-C3 (@ 8.4 Hz)/Coh. O2-F3 (@ 2.8 Hz); Coh. C4-F3 (@ 19.6 Hz)/Coh. O2-F4 (@ 12.8 Hz); Coh. F3-F4 (@20.4 Hz)/Coh. C3-F4 (@ 7.6 Hz); Coh. O1-C4 (@ 20 Hz)/Coh. C3-F4 (@ 6.8 Hz); Coh. C3-F3 (@ 0.2 Hz)/Coh. O1-F3 (@ 6.8 Hz); Coh. O1-C4 (@ 20.0 Hz)/Coh. O1-F3 (@ 45.6 Hz); Coh. C3-C4 (@ 20.6 Hz)/Coh. C3-F4 (@ 38.8 Hz); and Coh. C3-C4 (@ 20.0 Hz)/Coh. O2-C3 (@ 6.8 Hz); and a combination thereof.

In another embodiment of the method, examples of the coherence values for the sleep stage corresponding to awake period with lights off and before falling asleep (W) and for frequency band from brain wave patterns recorded at two scalp locations include, but are not limited to, Coh. O1-C3 (8.4-9.4 Hz), Coh. O2-F4 (12.6-13.6 Hz), Coh. O1-C3 (0.6-1.6 Hz), Coh. O1-F3 (6.8-7.8 Hz), Coh. O2-C3 (19.8-20.8 Hz), Coh. C3-F4 (7.4-8.4 Hz), Coh. F3-F4 (20.4-21.4 Hz), Coh. C3-F4 (7.6-8.6 Hz), Coh. O1-O2 (20-21 Hz), Coh. C3-F4 (49.8-50.8 Hz), Coh. C3-C4 (20-21 Hz), Coh. O1-F3 (45.6-46.6 Hz), Coh. C3-F4 (20.4-21.4 Hz); and Coh. O2-F3 (40-41 Hz); and a combination thereof.

In another embodiment of the method, examples of the coherence value ratio useful as a marker for the particular sleep stage corresponding to awake period with lights off and before falling asleep (W) for coherence values obtained from single frequency include, but are not limited to, Coh. O1-C3 (@ 8.4 Hz)/Coh. O2-F3 (@ 2.8 Hz), Coh. C4-F3 (@ 19.6 Hz)/Coh. O2-F4 (@ 12.8 Hz), Coh. F3-F4 (@ 20.4 Hz)/Coh. C3-F4 (@7.6 Hz), Coh. O1-C4 (@ 20 Hz)/Coh. C3-F4 (@ 6.8 Hz), Coh. C3-F3 (@ 0.2 Hz)/Coh. O1-F3 (@6.8 Hz), Coh. O1-C4 (@20.0 Hz)/Coh. O1-F3 (@ 45.6 Hz), Coh. C3-C4 (@ 20.6 Hz)/Coh. C3-F4 (@ 38.8 Hz); and Coh. C3-C4 (@ 20.0 Hz)/Coh. O2-C3 (@ 6.8 Hz), and a combination thereof.

In another embodiment of the method, examples of the coherence value ratio useful as a marker for the particular sleep stage corresponding to awake period with lights off and before falling asleep (W) for coherence values obtained from frequency band include, but are not limited to, Coh. O1-C3 (8.4-9.4 Hz)/Coh. O2-F4 (12.6-13.6 Hz), Coh. O1-C3 (0.6-1.6 Hz)/Coh. O1-F3 (6.8-7.8 Hz), Coh. O2-C3 (19.8-20.8 Hz)/Coh. C3-F4 (7.4-8.4 Hz), Coh. F3-F4 (20.4-21.4 Hz)/Coh. C3-F4 (7.6-8.6 Hz), Coh. O1-O2 (20-21 Hz)/Coh. C3-F4 (49.8-50.8 Hz), Coh. C3-C4 (20-21 Hz)/Coh. O1-F3 (45.6-46.6 Hz); and Coh. C3-F4 (20.4-21.4 Hz)/Coh. O2-F3 (40-41 Hz), and a combination thereof.

In another embodiment of the method, a neuromarker for PTSD symptom severity, based on single marker or single coherence value ratio for the particular sleep stage corresponding to awake period with lights off and before falling asleep (W) from single frequency, is Coh. C3-C4 (@ 20.0 Hz)/Coh. O2-C3 (@ 6.8 Hz).

In another embodiment of the method, examples of coherence values for the sleep stage corresponding to stage II sleep (S2) and for single frequency of brain wave patterns recorded at two scalp locations include, but are not limited to, Coh. O1-C3 (@ 0.6 Hz); Coh. O2-C4 (@ 1.0 Hz); Coh. C3-C4 (@ 19.8 Hz); Coh. F3-F4 (@ 0.6 Hz); Coh. O1-C4 (@ 5.0 Hz); Coh. O2-C3 (@ 6.4 Hz); Coh. O2-C3 (@ 19.8 Hz); Coh. O2-F3 (@ 11.8 Hz); Coh. C3-F4 (@ 37.8 Hz); Coh. C4-F3 (@ 39.6 Hz); Coh. C3-F4 (@ 20 Hz); and Coh. O2-F3 (@ 10 Hz); and a combination thereof.

In another embodiment of the method, examples of coherence values for the sleep stage corresponding to stage II sleep (S2) and for frequency band from brain wave patterns recorded at two scalp locations include, but are not limited to, Coh. C3-F3 (0.2-1.2 Hz); Coh. O1-F3 (48.2-49.2 Hz); Coh. O1-F4 (30.6-31.6 Hz); Coh. O2-F3 (29.4-30.4 Hz); Coh. O2-C3 (19.8-20.8 Hz); Coh. O2-F3 (11.8-12.8 Hz); Coh. C3-F4 (37.8-38.8 Hz); and Coh. C4-F3 (39.6-40.6 Hz); and a combination thereof.

In another embodiment of the method, examples of the coherence value ratio useful as a marker for the sleep stage corresponding to stage II sleep (S2) for coherence values obtained from single frequency include, but are not limited to, Coh. O1-C3 (@ 0.6 Hz)/Coh. O2-C4 (@ 1.0 Hz); Coh. C3-C4 (@ 19.8 Hz)/Coh. F3-F4 (@ 0.6 Hz); Coh. O1-C4 (@ 5.0 Hz)/Coh. O2-C3 (@ 6.4 Hz); Coh. O2-C3 (@ 19.8 Hz)/Coh. O2-F3 (@ 11.8 Hz); Coh. C3-F4 (@ 37.8 Hz)/Coh. C4-F3 (@ 39.6 Hz); and Coh. C3-F4 (@ 20 Hz)/Coh. O2-F3 (@ 10 Hz); and a combination thereof.

In another embodiment of the method, examples of the coherence value ratio useful as a marker for the sleep stage corresponding to stage II sleep (S2) for coherence values obtained from frequency band include, but are not limited to, Coh. C3-F3 (0.2-1.2 Hz)/Coh. O1-F3 (48.2-49.2 Hz); Coh. O1-F4 (30.6-31.6 Hz)/Coh. O2-F3 (29.4-30.4 Hz); Coh. O2-C3 (19.8-20.8 Hz)/Coh. O2-F3 (11.8-12.8 Hz); and Coh. C3-F4 (37.8-38.8 Hz)/Coh. C4-F3 (39.6-40.6 Hz); and a combination thereof.

In another embodiment of the method, a neuromarker for PTSD symptom severity, based on single marker or single coherence value ratio for the particular sleep stage corresponding to stage 2 sleep (S2) from single frequency is Coh. C3-F4 (@ 20 Hz)/Coh. O2-F3 (@ 10 Hz).

In one embodiment of the method, mathematical combination of two or more markers of steps a) dividing one coherence value by a second coherence value so as to obtain a coherence value ratio useful as a marker, which may serve as a neuromarker for PTSD symptom severity or may be combined with other markers to produce a neuromarker for PTSD symptom severity; b) repeating step (a) to obtain additional coherence value ratios, for a particular sleep stage is or comprises a combination of markers using multiple linear regression.

In another embodiment of the method, mathematical combination of two or more markers for the particular sleep stage corresponding to awake period with lights off and before falling asleep (W) at single frequency is or comprises multiple linear regression of two or more markers. Examples of the two or more markers include, but are not limited to, Coh. O1-C3 (@ 8.4 Hz)/Coh. O2-F3 (@ 2.8 Hz); Coh. C4-F3 (@ 19.6 Hz)/Coh. O2-F4 (@ 12.8 Hz); Coh. F3-F4 (@ 20.4 Hz)/Coh. C3-F4 (@ 7.6 Hz); Coh. O1-C4 (@20 Hz)/Coh. C3-F4 (@ 6.8 Hz); Coh. C3-F3 (@0.2 Hz)/Coh. O1-F3 (@ 6.8 Hz); Coh. O1-C4 (@ 20.0 Hz)/Coh. O1-F3 (@ 45.6 Hz); and Coh. C3-C4 (@ 20.6 Hz)/Coh. C3-F4 (@ 38.8 Hz).

In another embodiment of the method, the neuromarker for PTSD symptom severity obtained from mathematical combination of two or more markers for the particular sleep stage corresponding to awake period with lights off and before falling asleep (W) at single frequency by multiple linear regression of two or more markers is or comprises all the following seven markers: Coh. O1-C3 (@ 8.4 Hz)/Coh. O2-F3 (@ 2.8 Hz); Coh. C4-F3 (@ 19.6 Hz)/Coh. O2-F4 (@ 12.8 Hz); Coh. F3-F4 (@ 20.4 Hz)/Coh. C3-F4 (@7.6 Hz); Coh. O1-C4 (@ 20 Hz)/Coh. C3-F4 (@ 6.8 Hz); Coh. C3-F3 (@0.2 Hz)/Coh. O1-F3 (@ 6.8 Hz); Coh. O1-C4 (@20.0 Hz)/Coh. O1-F3 (@ 45.6 Hz); and Coh. C3-C4 (@20.6 Hz)/Coh. C3-F4 (@ 38.8 Hz).

In another embodiment of the method, the value of the neuromarker for PTSD symptom severity, designated PTSD_Symptom_Wake_1 neuromarker, is the sum of: a) 4.96×[Coh. O1-C3 (@ 8.4 Hz)/Coh. O2-F3 (@ 2.8 Hz)]; b)−6.97×[Coh. C4-F3 (@ 19.6 Hz)/Coh. O2-F4 (@ 12.8 Hz)]; c) 9.93×[Coh. F3-F4 (@ 20.4 Hz)/Coh. C3-F4 (@ 7.6 Hz)]; d) 5.08×[Coh. O1-C4 (@ 20 Hz)/Coh. C3-F4 (@ 6.8 Hz)]; e) 22.89×[Coh. C3-F3 (@ 0.2 Hz)/Coh. O1-F3 (@ 6.8 Hz)]; f)−10.65×[Coh. O1-C4 (@ 20.0 Hz)/Coh. O1-F3 (@ 45.6 Hz)]; and g) 32.96×[Coh. C3-C4 (@ 20.6 Hz)/Coh. C3-F4 (@ 38.8 Hz)]; and h)−6.62.

In a further embodiment of the method, a greater value of a neuromarker indicates a more severe PTSD symptom than a lesser value.

In another embodiment of the method, mathematical combination of two or more markers for the particular sleep stage corresponding to awake period with lights off and before falling asleep (W) from coherence value ratios obtained from frequency band is or comprises multiple linear regression of two or more markers. Examples of the two or more markers include, but are not limited to, Coh. O1-C3 (8.4-9.4 Hz)/Coh. O2-F4 (12.6-13.6 Hz), Coh. O1-C3 (0.6-1.6 Hz)/Coh. O1-F3 (6.8-7.8 Hz), Coh. O2-C3 (19.8-20.8 Hz)/Coh. C3-F4 (7.4-8.4 Hz), Coh. F3-F4 (20.4-21.4 Hz)/Coh. C3-F4 (7.6-8.6 Hz), Coh. O1-O2 (20-21 Hz)/Coh. C3-F4 (49.8-50.8 Hz), Coh. C3-C4 (20-21 Hz)/Coh. O1-F3 (45.6-46.6 Hz); and Coh. C3-F4 (20.4-21.4 Hz)/Coh. O2-F3 (40-41 Hz).

In another embodiment of the method, the neuromarker for PTSD symptom severity obtained from mathematical combination of two or more markers for the particular sleep stage corresponding to awake period with lights off and before falling asleep (W) at frequency band by multiple linear regression of two or more markers is or comprises all the following seven markers; Coh. O1-C3 (8.4-9.4 Hz)/Coh. O2-F4 (12.6-13.6 Hz), Coh. O1-C3 (0.6-1.6 Hz)/Coh. O1-F3 (6.8-7.8 Hz), Coh. O2-C3 (19.8-20.8 Hz)/Coh. C3-F4 (7.4-8.4 Hz), Coh. F3-F4 (20.4-21.4 Hz)/Coh. C3-F4 (7.6-8.6 Hz), Coh. O1-O2 (20-21 Hz)/Coh. C3-F4 (49.8-50.8 Hz), Coh. C3-C4 (20-21 Hz)/Coh. O1-F3 (45.6-46.6 Hz); and Coh. C3-F4 (20.4-21.4 Hz)/Coh. O2-F3 (40-41 Hz).

In another embodiment of the method, the value of the neuromarker for PTSD symptom severity, designated PTSD_Symptom_Wake_2 neuromarker, is the sum of: a) 9.18×[Coh. O1-C3 (8.4-9.4 Hz)/Coh. O2-F4 (12.6-13.6 Hz)]; b) 9.96×[Coh. O1-C3 (0.6-1.6 Hz)/Coh. O1-F3 (6.8-7.8 Hz)]; c) 7.67×[Coh. O2-C3 (19.8-20.8 Hz)/Coh. C3-F4 (7.4-8.4 Hz)]; d) 10.98×[Coh. F3-F4 (20.4-21.4 Hz)/Coh. C3-F4 (7.6-8.6 Hz)]; e)−21.75×[Coh. O1-O2 (20-21 Hz)/Coh. C3-F4 (49.8-50.8 Hz)]; f) 19.66×[Coh. C3-C4 (20-21 Hz)/Coh. O1-F3 (45.6-46.6 Hz)]; g) 16.27×[Coh. C3-F4 (20.4-21.4 Hz)/Coh. O2-F3 (40-41 Hz)]; and h) 1.04. In a further embodiment of the method, a greater value of a neuromarker indicates a more severe PTSD symptom than a lesser value.

In one embodiment of the method, mathematical combination of two or more markers for the particular sleep stage corresponding to stage II sleep (S2) at single frequency is or comprises multiple linear regression of two or more markers. Examples of the two or more markers include, but are not limited to, Coh. O1-C3 (@ 0.6 Hz)/Coh. O2-C4 (@ 1.0 Hz); Coh. C3-C4 (@ 19.8 Hz)/Coh. F3-F4 (@ 0.6 Hz); Coh. O1-C4 (@ 5.0 Hz)/Coh. O2-C3 (@ 6.4 Hz); Coh. O2-C3 (@ 19.8 Hz)/Coh. O2-F3 (@ 11.8 Hz); and Coh. C3-F4 (@ 37.8 Hz)/Coh. C4-F3 (@ 39.6 Hz).

In a further embodiment of the method, the neuromarker for PTSD symptom severity obtained from mathematical combination of two or more markers for the particular sleep stage corresponding to stage II sleep (S2) at single frequency by multiple linear regression of two or more markers is or comprises all of the following five markers: Coh. O1-C3 (@ 0.6 Hz)/Coh. O2-C4 (@ 1.0 Hz); Coh. C3-C4 (@ 19.8 Hz)/Coh. F3-F4 (@ 0.6 Hz); Coh. O1-C4 (@ 5.0 Hz)/Coh. O2-C3 (@ 6.4 Hz); Coh. O2-C3 (@19.8 Hz)/Coh. O2-F3 (@ 11.8 Hz); and Coh. C3-F4 (@37.8 Hz)/Coh. C4-F3 (@39.6 Hz).

In another embodiment of the method, the value of the neuromarker for PTSD symptom severity, designated PTSD_Symptom_Stage2_1 neuromarker, is the sum of: a) 43.40×[Coh. O1-C3 (@ 0.6 Hz)/Coh. O2-C4 (@ 1.0 Hz)]; b)−6.95×[Coh. C3-C4 (@ 19.8 Hz)/Coh. F3-F4 (@ 0.6 Hz)]; c) 42.77×[Coh. O1-C4 (@ 5.0 Hz)/Coh. O2-C3 (@ 6.4 Hz)]; d) 7.52×[Coh. O2-C3 (@ 19.8 Hz)/Coh. O2-F3 (@ 11.8 Hz)]; e) 82.32×[Coh. C3-F4 (@ 37.8 Hz)/Coh. C4-F3 (@ 39.6 Hz)]; and f)−125.09.

In another embodiment of the method, mathematical combination of two or more markers for the particular sleep stage corresponding to stage II sleep (S2) from coherence value ratios obtained from frequency band is or comprises multiple linear regression of two or more markers. Examples of the two or more markers include, but are not limited to, Coh. C3-F3 (0.2-1.2 Hz)/Coh. O1-F3 (48.2-49.2 Hz), Coh. O1-F4 (30.6-31.6 Hz)/Coh. O2-F3 (29.4-30.4 Hz), Coh. O2-C3 (19.8-20.8 Hz)/Coh. O2-F3 (11.8-12.8 Hz); and Coh. C3-F4 (37.8-38.8 Hz)/Coh. C4-F3 (39.6-40.6 Hz).

In a further embodiment of the method, the neuromarker for PTSD symptom severity obtained from mathematical combination of two or more markers for the particular sleep stage corresponding to stage II sleep (S2) at frequency band by multiple linear regression of two or more markers is or comprises all of the following four markers: Coh. C3-F3 (0.2-1.2 Hz)/Coh. O1-F3 (48.2-49.2 Hz), Coh. O1-F4 (30.6-31.6 Hz)/Coh. O2-F3 (29.4-30.4 Hz), Coh. O2-C3 (19.8-20.8 Hz)/Coh. O2-F3 (11.8-12.8 Hz); and Coh. C3-F4 (37.8-38.8 Hz)/Coh. C4-F3 (39.6-40.6 Hz).

In another embodiment of the method, the value of the neuromarker for PTSD symptom severity, designated PTSD_Symptom_Stage2_2 neuromarker, is the sum of: a) 11.45×[Coh. C3-F3 (0.2-1.2 Hz)/Coh. O1-F3 (48.2-49.2 Hz)]; b) 63.1×[Coh. O1-F4 (30.6-31.6 Hz)/Coh. O2-F3 (29.4-30.4 Hz)]; c) 5.89×[Coh. O2-C3 (19.8-20.8 Hz)/Coh. O2-F3 (11.8-12.8 Hz)]; d) 57.14×[Coh. C3-F4 (37.8-38.8 Hz)/Coh. C4-F3 (39.6-40.6 Hz)]; and e)−89.8.

In another embodiment of the method, mathematical combination of two or more markers of steps a) dividing one coherence value by a second coherence value so as to obtain a coherence value ratio useful as a marker, which may serve as a neuromarker for diagnosing PTSD or may be combined with other markers to produce a neuromarker for diagnosing PTSD; b) repeating step a) to obtain additional coherence value ratios, from two or more sleep stages comprises a combination of markers using multiple linear regression.

In a further embodiment of the method, the combination of markers using multiple linear regression comprises multiple linear regression of markers from one sleep stage.

In another embodiment of the method, the mathematical combination comprises or further comprises an arithmetic operation, wherein the arithmetic operation is selected from the group consisting of addition, subtraction, division and multiplication.

In another embodiment of the method, the mathematical combination comprises multiplication of value of multiple linear regression of markers from one sleep stage with value of multiple linear regression of markers from a different sleep stage.

In another embodiment of the method, the mathematical combination is multiplication of value of multiple linear regression of markers from one sleep stage with value of multiple linear regression of markers from a different sleep stage.

In another embodiment of the method, neuromarker from mathematical combination of two or more markers of steps a) dividing one coherence value by a second coherence value so as to obtain a coherence value ratio useful as a marker, which may serve as a neuromarker for PTSD symptom severity or may be combined with other markers to produce a neuromarker for PTSD symptom severity; b) repeating step (a) to obtain additional coherence value ratios, from two or more sleep stages is a combination comprising a marker or combination of markers from a sleep stage corresponding to awake period with lights off and before falling asleep (W) and a marker or combination of markers from a sleep stage corresponding to stage II sleep (S2).

In another embodiment of the method, neuromarker from mathematical combination of two or more markers of steps a) dividing one coherence value by a second coherence value so as to obtain a coherence value ratio useful as a marker, which may serve as a neuromarker for diagnosing PTSD or may be combined with other markers to produce a neuromarker for diagnosing PTSD; b) repeating step a) to obtain additional coherence value ratios, from two or more sleep stages is a combination of neuromarker from sleep stage corresponding to awake period with lights off and before falling asleep (W) and a neuromarker from a sleep stage corresponding to stage II sleep (S2).

In one embodiment of the method, the neuromarker is from a mathematical combination of two or more markers from two or more sleep stages is a combination of neuromarker from sleep stage corresponding to awake period with lights off and before falling asleep (W) and a neuromarker from a sleep stage corresponding to stage II sleep (S2).

In another embodiment of the method, the neuromarker is from a sleep stage corresponding to awake period with lights off and before falling asleep (W) is selected from the group consisting of PTSD_Symptom_Wake_1 neuromarker and PTSD_Symptom_Wake_2 neuromarker.

In an additional embodiment of the method, the neuromarker is from a sleep stage corresponding to stage II sleep (S2) is selected from the group consisting of PTSD_Symptom_Stage2_1 neuromarker of and PTSD_Symptom_Stage2_2 neuromarker.

In a yet a further embodiment of the method, the neuromarker is from a mathematical combination of two or more markers is a combination of PTSD_Symptom_Wake_1 neuromarker and PTSD_Symptom_Stage2_1 neuromarker.

Additionally in one embodiment, the neuromarker is designated PTSD_Symptom_S2×W_1 neuromarker, is a product of PTSD_Symptom_Wake_1 neuromarker and PTSD_Symptom_Stage2_1 neuromarker. Further, PTSD_Symptom_Wake_1 neuromarker including [4.96×[Coh. O1-C3 (@ 8.4 Hz)/Coh. O2-F3 (@ 2.8 Hz)]—6.97×[Coh. C4-F3 (@ 19.6 Hz)/Coh. O2-F4 (@ 12.8 Hz)]+9.93×[Coh. F3-F4 (@ 20.4 Hz)/Coh. C3-F4 (@ 7.6 Hz)]+5.08×[Coh. O1-C4 (@20 Hz)/Coh. C3-F4 (@ 6.8 Hz)]+22.89×[Coh. C3-F3 (@0.2 Hz)/Coh. O1-F3 (@ 6.8 Hz)]—10.65×[Coh. O1-C4 (@ 20.0 Hz)/Coh. O1-F3 (@ 45.6 Hz)]+32.96×[Coh. C3-C4 (@ 20.6 Hz)/Coh. C3-F4 (@ 38.8 Hz)]—6.62] and [43.40× [Coh. O1-C3 (@ 0.6 Hz)/Coh. O2-C4 (@ 1.0 Hz)]—6.95×[Coh. C3-C4 (@ 19.8 Hz)/Coh. F3-F4 (@ 0.6 Hz)]+42.77×[Coh. O1-C4 (@ 5.0 Hz)/Coh. O2-C3 (@6.4 Hz)]+7.52×[Coh. O2-C3 (@ 19.8 Hz)/Coh. O2-F3 (@ 11.8 Hz)]+82.32× [Coh. C3-F4 (@37.8 Hz)/Coh. C4-F3 (@ 39.6 Hz)]—125.09].

In still a further embodiment of the method, the neuromarker is from a mathematical combination of two or more markers is a combination of PTSD_Symptom_Wake_2 neuromarker and PTSD_Symptom_Stage2_2 neuromarker.

In a further embodiment of the method, the neuromarker, designated PTSD_Symptom_S2×W_2 neuromarker, is a product of PTSD_Symptom_Wake_2 neuromarker and PTSD_Symptom_Stage2_2 neuromarker. Including PTSD_Symptom_Wake_2 neuromarker are [9.18×[Coh. O1-C3 (8.4-9.4 Hz)/Coh. O2-F4 (12.6-13.6 Hz)]+9.96×[Coh. O1-C3 (0.6-1.6 Hz)/Coh. O1-F3 (6.8-7.8 Hz)]+7.67×[Coh. O2-C3 (19.8-20.8 Hz)/Coh. C3-F4 (7.4-8.4 Hz)]+10.98×[Coh. F3-F4 (20.4-21.4 Hz)/Coh. C3-F4 (7.6-8.6 Hz)]—21.75×[Coh. O1-O2 (20-21 Hz)/Coh. C3-F4 (49.8-50.8 Hz)]+19.66×[Coh. C3-C4 (20-21 Hz)/Coh. O1-F3 (45.6-46.6 Hz)]+16.27×[Coh. C3-F4 (20.4-21.4 Hz)/Coh. O2-F3 (40-41 Hz)]+1.04] and [11.45×[Coh. C3-F3 (0.2-1.2 Hz)/Coh. O1-F3 (48.2-49.2 Hz)]+63.1×[Coh. O1-F4 (30.6-31.6 Hz)/Coh. O2-F3 (29.4-30.4 Hz)]+5.89× [Coh. O2-C3 (19.8-20.8 Hz)/Coh. O2-F3 (11.8-12.8 Hz)]+57.14×[Coh. C3-F4 (37.8-38.8 Hz)/Coh. C4-F3 (39.6-40.6 Hz)]—89.8].

In an additional embodiment, the neuromarker for PTSD severity symptom provides a measure of severity of PTSD symptom.

In a further embodiment, the severity of PTSD symptom comprises a self-report of PTSD symptoms. In another further embodiment, the self-report comprises PTSD symptoms and a check list. In still yet one embodiment, the check list comprises a list of PTSD symptoms and a PTSD symptom scale. In addition another embodiment, the list of PTSD symptoms comprises at least 5 PTSD symptoms. In another embodiment the list of PTSD symptoms comprises 20 PTSD symptoms.

In an embodiment of the invention, the PTSD symptoms are any of PTSD symptoms and combination thereof, as outlined in the Diagnostic and Statistical Manual of Mental Disorders, 5^{th} Edition (DSM-V). For example, the PTSD symptoms may comprise 20 symptoms of PCL-5 self-report, e.g., the 20 symptoms which are outlined in the Diagnostic and Statistical Manual of Mental Disorders, 5^{th} Edition (DSM-V). In some embodiments, the self-report is or comprises a 20-item self-report PTSD checklist assessing 20 PTSD symptoms as outlined in the Diagnostic and Statistical Manual of Mental Disorders, 5^{th} Edition (DSM-V). In another embodiment, a measure of severity of PTSD symptoms comprises PCL-5 PTSD checklist. For example, the PCL-5 PTSD checklist may include a 20-item self-report which assesses 20 PTSD symptoms outlined in the Diagnostic and Statistical Manual of Mental Disorders, 5^{th} Edition (DSM-V). Merely by way of example, the PCL-5 PTSD checklist may produce a PCL-5 total symptom severity score. For example, the PCL-5 total symptom severity score may range from 0 to 80. In an embodiment, the PCL-5 total symptom severity score of 33 is estimated as a diagnostic threshold for PTSD. In still yet another embodiment, a PTSD subject may have a PCL-5 total symptom severity score of 33 or higher.

In accordance with the practice of the invention, the neuromarker for PTSD symptom severity is correlated to a measure of severity of PTSD symptoms. For example, the neuromarker for PTSD symptom severity may be correlated to a PCL-5 total symptom severity score. In one embodiment, the neuromarker positively correlates with a measure of severity of PTSD symptoms or severity of PTSD symptom as measured by PCL-5 total symptom severity score. For example, the correlation is performed by multiple linear regression analysis.

In one embodiment, the neuromarker for PTSD symptom severity shows a positive correlation with an R² value of at least 0.6 with a highly significant F-value for a neuromarker of one coherence value ratio derived from a sleep stage corresponding to awake period with lights off and before falling asleep (W). In another embodiment, the neuromarker for PTSD symptom severity shows a positive correlation with an R² value of at least 0.47 with a highly significant F-value for a neuromarker of one coherence value ratio derived from a sleep stage corresponding to stage II sleep (S2). In yet another embodiment, the neuromarker for PTSD symptom severity shows a positive correlation with an R² value of at least 0.79 with a highly significant F-value for a neuromarker comprising two or more coherence value ratios derived from a sleep stage corresponding to awake period with lights off and before falling asleep (W).

In a further embodiment, the neuromarker for PTSD symptom severity shows a positive correlation with an R² value between 0.75 and 0.90 with a highly significant F-value for a neuromarker comprising two or more coherence value ratios derived from a sleep stage corresponding to awake period with lights off and before falling asleep (W). In yet a further embodiment, the neuromarker for PTSD symptom severity shows a positive correlation with an R² value of at least 0.53 with a highly significant F-value for a neuromarker comprising two or more coherence value ratios derived from a sleep stage corresponding to stage II sleep (S2). In yet still another embodiment, the neuromarker for PTSD symptom severity shows a positive correlation with an R² value of at least 0.73 with a highly significant F-value for a neuromarker comprising two or more coherence value ratios derived from a sleep stage corresponding to stage II sleep (S2). In an additional embodiment, the neuromarker for PTSD symptom severity shows a positive correlation with an R² value of at least 0.72 with a highly significant F-value for a neuromarker comprising two or more coherence value ratios derived from two or more sleep stages, wherein the sleep stages are selected from the group consisting of awake period with lights off and before falling asleep (W), stage I sleep, stage II sleep (S2), delta-wave or stable III sleep or rapid-eye-movement (REM) sleep.

In a further embodiment, the neuromarker for PTSD symptom severity shows a positive correlation with an R² value of at least 0.72 with a highly significant F-value for a neuromarker comprising two or more coherence value ratios derived sleep stages correspond to awake period with lights off and before falling asleep (W) and stage II sleep (S2). In yet another further embodiment, the neuromarker for PTSD symptom severity shows a positive correlation with an R² value of at least 0.72 with a highly significant F-value for a neuromarker comprising two or more coherence value ratios derived from analysis of single frequencies in two or more brain wave patterns from two or more scalp locations. In still yet a further embodiment, the neuromarker for PTSD symptom severity shows a positive correlation with an R² value of at least 0.53 with a highly significant F-value for a neuromarker comprising two or more coherence value ratios derived from analysis of 1 Hz frequency bands in two or more brain wave patterns obtained from two or more scalp locations.

In some embodiments, the neuromarker for PTSD symptom severity shows a positive correlation with an R² value of at least 0.79 with a highly significant F-value for a neuromarker comprising two or more coherence value ratios derived from analysis of 1 Hz frequency bands in two or more brain wave patterns obtained from two or more scalp locations. In still yet embodiment, the neuromarker for diagnosis of PTSD obtained by a mathematical combination of two or more coherence value ratios in a multiple regression analysis has regression statistics with an R² of at least 0.79 with a highly significant F-value for a neuromarker comprising two or more coherence value ratios. In a further embodiment, the neuromarker for diagnosis of PTSD obtained by a mathematical combination of two or more coherence value ratios in a multiple regression analysis has regression statistics with an R² of at least 0.83 with a highly significant F-value for a neuromarker comprising two or more coherence value ratios.

In addition, in one embodiment, the neuromarker for diagnosis of PTSD obtained by a mathematical combination of two or more coherence value ratios in a multiple regression analysis has regression statistics with an R² of at least 0.93 with a highly significant F-value for a neuromarker comprising two or more coherence value ratios. In still a further embodiment, the neuromarker for diagnosis of PTSD obtained by a mathematical combination of two or more coherence value ratios in a multiple regression analysis has regression statistics with an R² of at least 0.96 with a highly significant F-value for a neuromarker comprising two or more coherence value ratios. In another embodiment, the neuromarker for diagnosis of PTSD obtained by a mathematical combination of two or more coherence value ratios in a multiple regression analysis has regression statistics with an R² between 0.79 and 0.96 with a highly significant F-value for a neuromarker comprising two or more coherence value ratios.

In some embodiments of the method, the highly significant F-value is at a significance level of p-value less than 0.01. In some embodiments of the method, the highly significant F-value is at a significance level of p-value less than 0.001. In some embodiments of the method, the highly significant F-value is at a significance level of p-value less than 0.0001. In some embodiments of the method, the highly significant F-value is at a significance level of p-value less than 0.00001. In some embodiments of the method, the highly significant F-value is at a significance level of p-value less than 0.000001.

In the present invention, in one embodiment, the method comprises a) obtaining two or more brain wave patterns from a subject; b) determining a value for one or more neuromarker selected from the group of PTSD_DiagWake_1 neuromarker, PTSD_Diag_Wake_2 neuromarker PTSD_Diag_Stage2_1 neuromarker, PTSD_Diag_Stage2_2 neuromarker, PTSD_Diag_S2×W_1 neuromarker and PTSD_Diag_S2×W_2 neuromarker from the brain wave patterns, wherein PTSD_Diag_Wake_1 neuromarker is [−0.11×[Coh. O1-O2 (@ 0.6 Hz)/Coh. O1-F4 (@ 2.4 Hz)]—0.27×[Coh. C3-C4 (@ 23.8 Hz)/Coh. O2-C3 (@ 37.0 Hz)]+0.72× [Coh. F3-F4 (@ 8.6 Hz)/Coh. C3-F3 (@ 6.8 Hz)]−0.07×[Coh. F3-F4 (@ 7.4 Hz)/Coh. C3-F4 (@ 7.6 Hz)]—0.11×[Coh. F3-F4 (@ 16.8 Hz)/Coh. O1-F4 (@7.4 Hz)]—0.31×[Coh. O1-F4 (@ 4.2 Hz)/Coh. O2-F3 (@ 13.0 Hz)]+1.92]; c) the PTSD_Diag_Wake_2 neuromarker is [0.92×[Coh. F3-F4 (8.6-9.6 Hz)/Coh. C3-F3 (@ 6.8-7.8 Hz)]—0.27×[Coh. F3-F4 (@ 7.6-8.6 Hz)/Coh. O2-F3 (@ 7.4-8.4 Hz)]+1.16]; d) the PTSD_Diag_Stage2_1 neuromarker is [− 0.16×[Coh. O1-O2 (@6.2 Hz)/Coh. C3-C4 (@ 7.0 Hz)]+0.17×[Coh. C3-C4 (@ 11.2 Hz)/Coh. O2-C4 (@ 16.6 Hz)]—0.17×[Coh. C3-C4 (@ 11.0 Hz)/Coh. O2-C3 (@ 16.4 Hz)]—0.07× [Coh. C3-C4 (@ 0.6 Hz)/Coh. C3-F4 (@ 2.6 Hz)]—0.09×[Coh. O1-C3 (@ 6.6 Hz)/Coh. O2-C3 (@ 0.6 Hz)]—0.08×[Coh. O1-C3 (@0.6 Hz)/Coh. O1-F3 (@6.8 Hz)]—0.05×[Coh. C3-F3 (@ 11.8 Hz)/Coh. O2-F3 (@ 6.8 Hz)]—0.09×[Coh. C4-F4 (@ 41.4 Hz)/Coh. C3-F4 (@2.6 Hz)]—0.10×[Coh. C4-F4 (@ 13.4 Hz)/Coh. O1-F3 (@ 15.8 Hz)]—0.07×[Coh. O1-C4 (@ 7.0 Hz)/Coh. C3-F4 (@3.2 Hz]+2.71]; e) the PTSD_Diag_Stage2_2 neuromarker is [− 0.17×[Coh. O1-O2 (6.2-7.2 Hz)/Coh. C3-C4 (7.0-8.0 Hz)]—0.02×[Coh. C3-C4 (7.0-8.0 Hz)/Coh. F3-F4 (8.6-9.6 Hz)]+0.2305×[Coh. C3-C4 (11.2-12.20 Hz)/Coh. O2-C4 (16.6-17.6 Hz)]—0.25×[Coh. C3-C4 (11.0-12.0 Hz)/Coh. O2-C3 (16.4-17.4 Hz)]—0.09×[Coh. C3-F3 (19.4-20.4 Hz)/Coh. O2-C3 (0.6-1.6 Hz)]—0.12×[Coh. C4-F4 (41.4-42.4 Hz)/Coh. C3-F4 (2.6-3.6 Hz)]+2.40]; f) the PTSD_Diag_S2×W_1 neuromarker (product of PTSD_Diag_Wake_1 neuromarker and PTSD_Diag_Stage2_1 neuromarker) is [− 0.11×[Coh. O1-O2 (@ 0.6 Hz)/Coh. O1-F4 (@ 2.4 Hz)]—0.27× [Coh. C3-C4 (@ 23.8 Hz)/Coh. O2-C3 (@ 37.0 Hz)]+0.72×[Coh. F3-F4 (@ 8.6 Hz)/Coh. C3-F3 (@ 6.8 Hz)]—0.07×[Coh. F3-F4 (@ 7.4 Hz)/Coh. C3-F4 (@ 7.6 Hz)]-0.11×[Coh. F3-F4 (@ 16.8 Hz)/Coh. O1-F4 (@ 7.4 Hz)]—0.31×[Coh. O1-F4 (@ 4.2 Hz)/Coh. O2-F3 (@ 13.0 Hz)]+1.92]×[− 0.16×[Coh. O1-O2 (@ 6.2 Hz)/Coh. C3-C4 (@ 7.0 Hz)]+0.17×[Coh. C3-C4 (@ 11.2 Hz)/Coh. O2-C4 (@16.6 Hz)]—0.17×[Coh. C3-C4 (@ 11.0 Hz)/Coh. O2-C3 (@ 16.4 Hz)]—0.07× [Coh. C3-C4 (@0.6 Hz)/Coh. C3-F4 (@ 2.6 Hz)]—0.09×[Coh. O1-C3 (@6.6 Hz)/Coh. O2-C3 (@ 0.6 Hz)]—0.08×[Coh. O1-C3 (@ 0.6 Hz)/Coh. O1-F3 (@6.8 Hz)]—0.05×[Coh. C3-F3 (@ 11.8 Hz)/Coh. O2-F3 (@ 6.8 Hz)]—0.09×[Coh. C4-F4 (@ 41.4 Hz)/Coh. C3-F4 (@2.6 Hz)]—0.10×[Coh. C4-F4 (@ 13.4 Hz)/Coh. O1-F3 (@ 15.8 Hz)]—0.07×[Coh. O1-C4 (@7.0 Hz)/Coh. C3-F4 (@3.2 Hz]+2.71]; g) and the PTSD_Diag_S2×W_2 neuromarker (product of PTSD_Diag_Wake_2 neuromarker and PTSD_Diag_Stage2_2 neuromarker) is [0.92× [Coh. F3-F4 (8.6-9.6 Hz)/Coh. C3-F3 (@ 6.8-7.8 Hz)]—0.27×[Coh. F3-F4 (@ 7.6-8.6 Hz)/Coh. O2-F3 (@ 7.4-8.4 Hz)]+1.16]×[− 0.17×[Coh. O1-O2 (6.2-7.2 Hz)/Coh. C3-C4 (7.0-8.0 Hz)]—0.02×[Coh. C3-C4 (7.0-8.0 Hz)/Coh. F3-F4 (8.6-9.6 Hz)]+0.2305×[Coh. C3-C4 (11.2-12.20 Hz)/Coh. O2-C4 (16.6-17.6 Hz)]—0.25×[Coh. C3-C4 (11.0-12.0 Hz)/Coh. O2-C3 (16.4-17.4 Hz)]—0.09× [Coh. C3-F3 (19.4-20.4 Hz)/Coh. O2-C3 (0.6-1.6 Hz)]—0.12×[Coh. C4-F4 (41.4-42.4 Hz)/Coh. C3-F4 (2.6-3.6 Hz)]+2.40]; h) detecting post-traumatic stress disorder in the subject by determining if the value of the one or more neuromarkers is above a designated threshold, or is increased or decreased relative to a control value.

In the present invention, in yet another embodiment, the method comprises a) obtaining two or more brain wave patterns from a subject; b) determining a value for one or more neuromarker selected from the group of PTSD_Symptom_Wake_1 neuromarker, PTSD_Symptom_Wake_2 neuromarker PTSD_Symptom_Stage2_1 neuromarker, PTSD_Symptom_Stage2_2 neuromarker, PTSD_Symptom_S2×W_1 neuromarker and PTSD_Symptom_S2×W_2 neuromarker from the brain wave patterns, wherein PTSD_Symptom_Wake_1 neuromarker is [4.96×[Coh. O1-C3 (@ 8.4 Hz)/Coh. O2-F3 (@ 2.8 Hz)]—6.97×[Coh. C4-F3 (@ 19.6 Hz)/Coh. O2-F4 (@ 12.8 Hz)]+9.93×[Coh. F3-F4 (@ 20.4 Hz)/Coh. C3-F4 (@ 7.6 Hz)]+5.08×[Coh. O1-C4 (@ 20 Hz)/Coh. C3-F4 (@ 6.8 Hz)]+22.89×[Coh. C3-F3 (@ 0.2 Hz)/Coh. O1-F3 (@6.8 Hz)]—10.65×[Coh. O1-C4 (@ 20.0 Hz)/Coh. O1-F3 (@ 45.6 Hz)]+32.96× [Coh. C3-C4 (@ 20.6 Hz)/Coh. C3-F4 (@ 38.8 Hz)]—6.62]; b) PTSD_Symptom_Wake_2 neuromarker is [9.18×[Coh. O1-C3 (8.4-9.4 Hz)/Coh. O2-F4 (12.6-13.6 Hz)]+9.96×[Coh. O1-C3 (0.6-1.6 Hz)/Coh. O1-F3 (6.8-7.8 Hz)]+7.67×[Coh. O2-C3 (19.8-20.8 Hz)/Coh. C3-F4 (7.4-8.4 Hz)]+10.98× [Coh. F3-F4 (20.4-21.4 Hz)/Coh. C3-F4 (7.6-8.6 Hz)]—21.75×[Coh. O1-O2 (20-21 Hz)/Coh. C3-F4 (49.8-50.8 Hz)]+19.66×[Coh. C3-C4 (20-21 Hz)/Coh. O1-F3 (45.6-46.6 Hz)]+16.27×[Coh. C3-F4 (20.4-21.4 Hz)/Coh. O2-F3 (40-41 Hz)]+1.04]; c) the PTSD_Symptom_Stage2_1 neuromarker is [43.40×[Coh. O1-C3 (@ 0.6 Hz)/Coh. O2-C4 (@ 1.0 Hz)]—6.95×[Coh. C3-C4 (@ 19.8 Hz)/Coh. F3-F4 (@ 0.6 Hz)]+42.77×[Coh. O1-C4 (@ 5.0 Hz)/Coh. O2-C3 (@ 6.4 Hz)]+7.52×[Coh. O2-C3 (@ 19.8 Hz)/Coh. O2-F3 (@ 11.8 Hz)]+82.32×[Coh. C3-F4 (@ 37.8 Hz)/Coh. C4-F3 (@ 39.6 Hz)]—125.09]; d) the PTSD_Symptom_Stage2_2 neuromarker is: [11.45×[Coh. C3-F3 (0.2-1.2 Hz)/Coh. O1-F3 (48.2-49.2 Hz)]+63.1×[Coh. O1-F4 (30.6-31.6 Hz)/Coh. O2-F3 (29.4-30.4 Hz)]+5.89×[Coh. O2-C3 (19.8-20.8 Hz)/Coh. O2-F3 (11.8-12.8 Hz)]+57.14×[Coh. C3-F4 (37.8-38.8 Hz)/Coh. C4-F3 (39.6-40.6 Hz)]—89.8]; e) the PTSD_Symptom_S2×W_1 neuromarker (product of PTSD_Symptom_Wake_1 neuromarker and PTSD_Symptom_Stage2_1 neuromarker) is [4.96×[Coh. O1-C3 (@ 8.4 Hz)/Coh. O2-F3 (@ 2.8 Hz)]—6.97×[Coh. C4-F3 (@ 19.6 Hz)/Coh. O2-F4 (@ 12.8 Hz)]+9.93×[Coh. F3-F4 (@ 20.4 Hz)/Coh. C3-F4 (@ 7.6 Hz)]+5.08×[Coh. O1-C4 (@ 20 Hz)/Coh. C3-F4 (@ 6.8 Hz)]+22.89×[Coh. C3-F3 (@ 0.2 Hz)/Coh. O1-F3 (@ 6.8 Hz)]—10.65×[Coh. O1-C4 (@ 20.0 Hz)/Coh. O1-F3 (@ 45.6 Hz)]+32.96×[Coh. C3-C4 (@ 20.6 Hz)/Coh. C3-F4 (@ 38.8 Hz)]—6.62]×[43.40× [Coh. O1-C3 (@ 0.6 Hz)/Coh. O2-C4 (@ 1.0 Hz)]—6.95×[Coh. C3-C4 (@ 19.8 Hz)/Coh. F3-F4 (@ 0.6 Hz)]+42.77×[Coh. O1-C4 (@ 5.0 Hz)/Coh. O2-C3 (@6.4 Hz)]+7.52×[Coh. O2-C3 (@ 19.8 Hz)/Coh. O2-F3 (@ 11.8 Hz)]+82.32× [Coh. C3-F4 (@ 37.8 Hz)/Coh. C4-F3 (@ 39.6 Hz)]—125.09]; f) and the PTSD_Symptom_S2×W_2 neuromarker (product of PTSD_Symptom_Wake_2 neuromarker and PTSD_Symptom_Stage2_2 neuromarker) is [9.18×[Coh. O1-C3 (8.4-9.4 Hz)/Coh. O2-F4 (12.6-13.6 Hz)]+9.96×[Coh. O1-C3 (0.6-1.6 Hz)/Coh. O1-F3 (6.8-7.8 Hz)]+7.67×[Coh. O2-C3 (19.8-20.8 Hz)/Coh. C3-F4 (7.4-8.4 Hz)]+10.98×[Coh. F3-F4 (20.4-21.4 Hz)/Coh. C3-F4 (7.6-8.6 Hz)]—21.75×[Coh. O1-O2 (20-21 Hz)/Coh. C3-F4 (49.8-50.8 Hz)]+19.66×[Coh. C3-C4 (20-21 Hz)/Coh. O1-F3 (45.6-46.6 Hz)]+16.27×[Coh. C3-F4 (20.4-21.4 Hz)/Coh. O2-F3 (40-41 Hz)]+1.04]×[11.45×[Coh. C3-F3 (0.2-1.2 Hz)/Coh. O1-F3 (48.2-49.2 Hz)]+63.1×[Coh. O1-F4 (30.6-31.6 Hz)/Coh. O2-F3 (29.4-30.4 Hz)]+5.89×[Coh. O2-C3 (19.8-20.8 Hz)/Coh. O2-F3 (11.8-12.8 Hz)]+57.14×[Coh. C3-F4 (37.8-38.8 Hz)/Coh. C4-F3 (39.6-40.6 Hz)]−89.8]; g) the value so obtained relates to severity of PTSD symptom, h) determining severity of PTSD symptoms from brain wave patterns of a PTSD patient. For example, the higher a value signifies greater the severity of PTSD symptom.

Merely by way of example, the value obtained relates or correlates to severity of PTSD symptoms of PCL-5 total symptom severity score system or a standard PSTD symptom score system. In one further embodiment of the method, the correlation is a positive correlation. In another further embodiment of the method, the correlation is a negative correlation.

In another further embodiment, the PCL-5 total symptom severity score system is based on or comprises 20-item self-report which assesses 20-item PTSD symptoms outlines in the Diagnostic and Statistical Manual of Mental Disorders, 5^{th} Edition (DSM-V).

In some of the embodiments of the method, value of at least one slope of the markers comprising the neuromarker vary by or within 10%. In some embodiments of the method, ratio of slopes of at least two markers of the neuromarker are maintained within +10%. In some embodiments of the method, intercept may vary so as to change PTSD symptom severity value by a constant or fixed value. In some embodiments of the method, mathematical expression is changed by multiplication, division, addition or subtraction by a positive or negative number.

In the present invention one aspect provides a method for determining changes in PTSD symptom severity in a PTSD subject. In one embodiment, the subject comprises a) measuring severity of PTSD symptom, so as to obtain a value for one or more PTSD neuromarker for PTSD symptom severity; b) measuring severity of PTSD symptom at a second time point; c) comparing value of (a) with value of (b) to determine if the two values are the same or different, wherein the same values indicate no change in PTSD symptom severity, increased in value of measurement in (b) indicates increased severity, decreased in value of measurement in (b) indicates decreased severity, and magnitude of difference indicates magnitude of change in PTSD symptom severity, thereby, determining changes in PTSD symptom severity in a PTSD subject.

In the present invention one aspect provides a method for diagnosing PTSD and assessing severity of PTSD symptoms in a subject. In one embodiment, the subject comprises a) detecting PTSD in a subject by a method of the invention; b) determining severity of PTSD symptoms in the subject so detected in step (a) by a method of the invention.

In the present invention, one aspect provides a method for determining efficacy of a therapy or drug in treating PTSD in a PTSD subject. In one embodiment the subject comprises a) administering the therapy or drug to a PTSD subject; b) detecting presence of PTSD in the subject by the method of the invention to determine if PTSD persists in the subject; and/or c) measuring severity of PTSD symptoms by the method of the invention, to determine if severity of PTSD symptoms is reduced, thereby, determining efficacy of a therapy or drug in treating PTSD in a PTSD subject.

In the present invention one aspect provides a method for determining efficacy of a therapy or drug in preventing PTSD in a subject. In one embodiment the subject comprises a) administering the therapy or drug to a subject without PTSD or prior history of PTSD; b) exposing the subject PTSD conditions; c) detecting presence of PTSD in the subject by the method of the invention to detect presence of PTSD in the subject with the finding of no PTSD indicative of a therapy or drug in preventing PTSD in a subject; thereby, determining efficacy of a therapy or drug in preventing PTSD in a PTSD subject.

In the present invention one aspect provides a method for identifying a therapy or drug in ameliorating symptoms of PTSD in a PTSD subject. In one embodiment the subject comprises a) administering the therapy or drug to a PTSD subject; b) measuring severity of PTSD symptoms by the method of the invention, to determine if severity of PTSD symptoms is reduced by the administration of the therapy or drug to the PTSD subject, thereby, identifying a therapy or drug in ameliorating symptoms of PTSD in a PTSD subject.

In the present invention one aspect provides a method for determining presence of PTSD and PTSD symptom severity in a subject. In one embodiment, a neuromarker value above or below a threshold value establishes presence of PTSD and severity of PTSD symptom correlates with neuromarker value above or below the threshold in the subject identified to have PTSD or likely to have PTSD. In another embodiment, a neuromarker value above or below a threshold value establishes presence of PTSD and severity of PTSD symptom correlates with neuromarker value above or below the threshold in the subject identified to have PTSD or likely to have PTSD. In another embodiment, a neuromarker value above about 33 establishes presence of PTSD and severity of PTSD symptom correlates with neuromarker value beyond about 33 in the subject identified to have PTSD or likely to have PTSD.

Association of PTSD with Sleep Abnormalities

As described herein, the inventors have developed PTSD neuromarkers that are more sensitive and more useful, based on quantitative EEG analysis. This was primarily done by focusing on innovative brain signal analysis and brain signal feature selection during sleep. The rationale for focusing on the sleep state for diagnostic neuromarkers is the prevalence of sleep disturbances in PTSD patients [7, 8, 13, 14]. As used herein, a “patient” or “subject” refers to an individual, particularly a human individual, for which a neuromarker as described herein may be employed. A patient or subject may refer to an individual having PTSD, or an individual not having PTSD, also referred to herein as a “normal.”

More than 70% of civilians and veterans with PTSD have reported persistent and severe nightmares and disturbed sleep, particularly of the insomnia type [9]. Insomnia is defined as difficulty in initiating or maintaining sleep, waking up too early, or having non-restorative sleep despite adequate opportunity for sleep, which can lead to poor daytime functioning due to fatigue or mood disorder [10].

Further evidence of strong association between PTSD and sleep disturbance was provided by Germaine et al., who studied 367 people with PTSD and found that the severity of PTSD was most closely associated with the severity of sleep disturbances [17]. More importantly, evidence suggests that sleep disturbances based on subjective reports of insomnia symptoms and polysomnography (PSG) appear before the onset of PTSD and therefore, disturbed sleep could be a risk factor for development of PTSD [18-20]. In fact, a growing body of evidence shows that disturbed sleep is more than a secondary symptom of PTSD but rather seems to be a core feature [21, 22]. This is further supported by evidence that sleep disturbances are a frequent residual complaint after successful PTSD treatment [23]. In contrast, both sleep disturbances and severity of PTSD symptoms are alleviated following treatments that focus only on sleep disorders [24].

Association of PTSD with abnormal sleep quality and quantity has also been shown in a number of studies using the macro structure of sleep measured via polysomnography (PSG). Excessive stage I sleep was seen in a meta-analysis of PSG studies comparing PTSD and control groups [25]. In addition to increased presence of stage I sleep, the meta-analysis showed diminished slow wave sleep and higher REM density in patients. Other studies implicate REM abnormalities in PTSD patients, particularly excessive REM-to-wake and REM-to-stage-I transitions, as the main sleep abnormality in PTSD patients, consistent with the increased amount of stage I sleep reported in the meta-analysis described above [26, 27]. Germaine [28] also reported that disturbed REM and non-REM sleep in PTSD patients evaluated by PSG contributes to maladaptive stress and may be a modifiable risk factor for poor psychiatric outcome.

Stages of Sleep

As used herein, a “sleep stage” refers to a period of time during sleep in which the subject's awareness and brain wave patterns change in a predictable pattern. In accordance with the invention, 5 sleep states are defined: (1) Awake, (2) Stage I Sleep, (3) Stage 2 Sleep, (4) Delta-wave or stage 3 sleep, and (5) rapid-eye-movement (REM) sleep. These stages progress cyclically from 1 through REM then begin again with stage 1. A complete sleep cycle may take an average of 90 to 110 minutes.

Awake refers to the time when a person is not asleep. Sleep stage I is characterized by light sleep in which the subject may drift in and out of sleep and can be awakened easily. In this stage, the eyes move slowly and muscle activity slows. During this stage, many subjects experience sudden muscle contractions preceded by a sensation of falling.

In sleep stage 2, eye movement stops and brain waves become slower with only an occasional burst of rapid brain waves. Stage 3 is characterized by extremely slow brain waves called delta waves, referred to as deep sleep or delta sleep. In this stage, there is no eye movement or muscle activity, and it is very difficult to wake someone.

REM sleep is characterized by more rapid irregular and shallow breathing, rapid jerking of the eyes, and temporary paralysis of limb muscles. Brain waves during this stage increase to levels experienced when a person is awake, heart rate increases, blood pressure rises, and the body loses some ability to regulate temperature. Most dreams occur in REM sleep. Most people experience three to five intervals of REM sleep each night. The first sleep cycles each night have relatively short REM sleeps and long periods of deep sleep but later in the night, REM periods lengthen and deep sleep time decreases.

For further discussion of sleep stages and sleep analysis, see Berry R B, Brooks R, Gamaldo C E, Harding S M, Marcus C L and Vaughn B V for the American Academy of Sleep Medicine. The AASM Manual for the Scoring of Sleep and Associated Events: Rules, Terminology and Technical Specifications, Version 2.2. aasm.org.

As used herein, a “fluctuation” or “transition” refers to the movement of a subject from one sleep stage to another. Transition among each sleep stage is marked by subtle changes in bodily function. Transitions can be monitored by the methods of the invention and using parameters set forth herein.

Analysis of Brain Wave Patterns

In accordance with the invention, a standard sleep analysis may be used in which electrodes are used to pick up brain wave signals (Nunez P L, Srinivasan R, Westdorp A F, Wijesinghe R S, Tucker D M, Silberstein R B, Cadusch P J (1997). EEG coherency. I. Statistics, reference electrode, volume conduction, Laplacians, cortical imaging, and interpretation at multiple e scales. Electroencephalogr Clin Neurophysiol; 103: 499-515). Electrodes may be placed at one or several locations on the scalp or body in order to detect EEG or brain wave signals. Locations for an electrode may include frontal, parietal, anterior, central and occipital (0).

The subject's brain waves or EEG signals may be recorded and analyzed during the test time period. As used herein, the “test time period” may be defined as the period of time in which the subject's brain waves signals are measured or recorded. For the purposes of the present invention, a standard sleep study may comprise any time period appropriate for obtaining sufficient data relating to brain wave patterns, for example about 5 hours or more, including 5 hours, 6 hours, 7 hours, 8 hours, 9 hours, 10 hours, 11 hours, 12 hours, or the like. In some specific embodiments, a minimum time period of 6 hours may be desired in order to obtain the desired data. Generally, a test time period may correspond to the time period in which the subject or patient is hooked up or connected to the electrodes for recording of brain wave signals, or to the time period necessary for a subject's brain wave signals to obtain a consistent pattern. The electrodes are connected to appropriate electronic machinery for recording and analysis of the brain waves.

During the testing of a subject for analysis of brain wave patterns, the subject's brain waves or EEG signals are collected and analyzed to obtain data as described herein for each sampling moment or time segment. The data may be collected through conventional recorders, analog signal processors, or other devices appropriate for use with the invention and analyzed after collection. Alternatively, the collection and analysis of the brain waves or EEG signals may be carried out concurrently or simultaneously using any means appropriate. In one embodiment of the present invention, a processor or computer may receive digitized signals based on analog signals from the sensor used to measure the subject's brain waves or EEG signals. The brain waves or EEG signals may then be filtered using, for example, a digital filter. Such methods may be followed by artifact detection and removal.

In some embodiments, the technology associated with either laboratory or home-based sleep monitoring has undergone a tremendous advancement in the past few years such that current cost-effective, miniaturized, and easy-to-wear/sleep monitors can be self-administered by the patients/care-givers in the home environment while yielding laboratory grade EEG data.

Applications useful in accordance with the invention may include, but are not limited to, use with alertness devices, sleep analysis devices, anesthesia monitors, psychiatric and hypnosis medication monitoring, and for looking at the effect of various other therapies. In any of these types of applications, a subject's brain waves or EEG signals may be collected and analyzed as described herein.

EEG frequency data in accordance with the invention may be any value from 0.1 to 128 Hz, either at discrete values, for example 0.1 Hz, 0.2 Hz, 0.3 Hz, 0.4 Hz, 0.5 Hz, 1 z, 1.5 Hz, 2 Hz, 2.5 Hz, 3 Hz, 4 Hz, 5 Hz, 6, Hz, 7 Hz, 8 Hz, 9 Hz, 10 Hz, 15 Hz, 20 Hz, 25 Hz, 30 Hz, 35 Hz, 40 Hz, 45 Hz, 50 Hz, 55 Hz, 60 Hz, 65 Hz, 70 Hz, 75 Hz, 80 Hz, 85 Hz, 90 Hz, 95 Hz, 100 Hz, 105 Hz, 110 Hz, 115 Hz, 120 Hz, 125 Hz, 126 Hz, 127 Hz, 128 Hz, 129 Hz, 130 Hz, or the like. In other embodiments, EEG frequency data may be a delta wave (1-4 Hz), a theta wave (4-7 Hz), an alpha wave (1-8 Hz), or the like. In further embodiments, such frequencies may be obtained or detected during wake or during any sleep stages, and not restricted to a particular sleep stage. Sleep studies in accordance with the invention may also be performed during the day, such as during a brief nap, and the neuromarkers can be just as easily computed during that time. Thus, measurement may be obtained even in a clinic or patient examination room in the hospital.

In some embodiments, brain wave data collected and monitored in accordance with the invention may be between about 8-12 Hz corresponding to sleep stage I alpha waves, between about 13-16 Hz corresponding to sleep stage 1 sigma waves, between about 16-25 Hz corresponding to sleep stage I beta waves, between about 35-45 Hz corresponding to sleep stage 1 gamma waves, between about 13-16 Hz corresponding to sleep stage 2 sigma waves, between about 35-45 Hz corresponding to sleep stage 2 gamma waves, or between about 13-16 Hz corresponding to REM sleep. As those skilled in the art will appreciate, the boundaries between these components are somewhat arbitrary, and thus, the foregoing delineations are intended to be exemplary and not limiting. Furthermore, use of other components, whether now known or later discovered, are within the scope of the invention.

As used herein, a “sleep study parameter” refers to a characteristic or trait for which a value or measurement may be observed or recorded and that provides diagnostic or reference value for PTSD. Sleep study parameters useful for the invention may include, but are not limited to measurements of electrical and muscular states such as EEG, electro-oculography (EOG), and surface electromyography (EMG), sleep latency and arousals, horizontal and vertical eye movements, presence or absence of atonia, airflow resistance m the airways, electrocardiography, pulse oximetery, respiratory effort (thoracic and abdominal), end tidal or transcutaneous C0₂, snoring, limb movement, core body temperature, incident light intensity, and/or esophageal pressure and pH. One of skill in the art will recognize that other parameters may be used to assess sleep or brainwave patterns as appropriate, or that some parameters may be added or eliminated according to the needs of the individual patient or study.

In other embodiments of the present invention, the methods described above may be used to prevent PTSD or treat a subject for PTSD. For example, the subject's brain wave signals may be quantitatively analyzed to determine if the subject has PTSD as described herein, or by some other method that henceforth becomes known to those skilled in the art. If the subject is found to have PTSD, a physician or technician may therapeutically treat the subject by providing psycho-therapy, including exposure-based therapy and extinction of fear memory training, or by administering or prescribing a medication to the subject in order to treat PTSD in the patient one or more symptoms of PTSD in the patient. Subsequent re-testing or re-analysis of the subject may be performed in order to estimate or determine the extent of improvement of PTSD in the subject after a reasonable period of time to allow for the therapy to have an ameliorative effect. Determination that the subject requires further treatment or assessment may be performed based on comparison of a data profile obtained for a particular subject after treatment or intervention with that of the same subject before treatment or intervention. Comparison may also be made of a particular subject with PTSD to a subject without PTSD. From this comparison, a decision may be made to increase, reduce, or eliminate a therapeutic treatment, or to add additional therapies to attain a data profile for a subject with PTSD similar to or equivalent to that of a normal subject. In certain specific embodiments, consideration may be made for subjects to be analyzed or treated as described herein based on medications the subject may be taking for related or unrelated conditions, for example medications that may alter the brain wave pattern of a subject.

In other embodiments, data obtained from a particular subject suspected to have PTSD but not exhibiting symptoms may be compared to an individual known to be without PTSD at different time points in order to obtain multiple points of data along a time line. In such a case, observation of an increase in one or more neuromarkers of the invention may serve as a signal of onset of PTSD. In this manner, early diagnosis or determination of PTSD in a subject may be obtained and treatment initiated such that onset of symptoms is delayed or prevented.

Development of Sleep-Related Neuromarkers

As described herein, the state of sleep and the quality/quantity thereof is highly correlated to the presence and severity of PTSD. Brain function during sleep was utilized as the platform for developing objective PTSD neuromarkers based on EEG micro-analysis, a novel approach compared to current methods. As used a “neuromarker” or “marker” refers to a characteristic that is useful for diagnosing the presence or absence of PTSD, or the severity of PTSD. A neuromarker in accordance with the invention may be a sleep study parameter described herein, or may be any other parameter that may be obtained using the methods of the invention.

Standard measurement of clinical sleep stages is based on classifying the macro-structure (30-second epoch) of EEG and other biosignals, for example electro-occulograms and chin electromyograms, into the conventional stages of sleep, such as ‘active rapid-eye-movement’ (REM) sleep and 3 stages of non-REM sleep, ‘Stage I’, ‘Stage 2’, and ‘Stage 3’, also known as slow-wave or delta sleep. Currently available methods for analysis of sleep patterns have focused on more gross or ‘macro’ measures, such as time spent in each of the sleep stages, the presence or absence of a given sleep stage, the frequency or sequence of occurrence of each of these sleep stages, the degree of sleep fragmentation, and fluctuation patterns across sleep stages. These measures have been used to compare these macro-structures of sleep in PTSD patients with those of normal individuals. The invention utilizes standard sleep staging known in the art for identifying the macro sleep architecture. In addition to this, the methods of the invention evaluate a number of informative brain signal features according to the micro-dynamics of an EEG (periods of 5 seconds or less) within each of the clinical sleep stages, described in detail herein. Thus, a novel neuromarker of the invention was developed and is based on both micro and macro sleep structures.

EEG micro-analysis consists of performing coherence computation between specific EEG pairs to derive a set of neuromarkers that reflect the degree of inter- and intra-hemispheric synchronization of EEG frequency bands over a specific time period within a specific sleep stage. Such an approach of combining micro-level quantitative EEG analysis (coherence) with macro-level sleep staging for analysis of brain function in human PTSD is novel over currently available methods known in the art, which either focus only on macro-sleep analysis [25-28], or employ EEG analysis during active awake periods as part of neuro-feedback treatment [29]. The present invention thus is novel over the art by providing quantitative and non-invasive neuromarkers that can be acquired and measured in a cost-effective and convenient manner.

Structure of Neuromarkers

As described herein, a novel class of neuromarkers is described that are based on the coherence and phase delays of pairs of scalp EEG electrodes. Coherences and phase delays have been used for assessment of brain activities of patients with traumatic brain injury [32, 33]. As used herein, “coherence” refers to a normalized quantity (index spans from 0 to 1) that reflects the degree of association or coupling of the power levels in a pair of EEG waveforms (from two scalp sites) and for a given frequency band (e.g., alpha band that covers EEG waveforms with frequencies from 8 to 12 cycles/second).

Formally, coherence is a generalization of correlation analysis and is computed as the magnitude of normalized cross-power spectrum [30] of a pair of simultaneously recorded EEGs from two separate scalp locations. Coherence reflects the degree of coupling and functional association between two brain regions [31] and can be computed for specific frequency bands of EEG pairs.

Associated with coherence level is phase delay, which essentially provides information about the time delay between the two EEG waveforms at a specific single frequency or a narrow-band frequency range. Phase delays essentially show the directed coherence and provide information about the time delay between the two EEG waveforms at a specific single frequency or a narrow-band frequency. The coherence and phase delays of the present invention may be computed on a micro-level, where the real and imaginary parts of the cross-spectra are utilized to compute the phase angles. Phase angles may be expressed herein as a fraction of (2*pi), where 2*pi corresponds to a whole cycle of a given frequency. For example, a phase angle of pi/2 is one-fourth of 2*pi, corresponding to 0.25 of a cycle. Thus, for a waveform with a frequency of 1 cycle/second, a pi/2 delay translates to one-fourth of a cycle or one-fourth of 1 second.

In accordance with the invention, a phase angle may thus be converted to a time measure (usually in units of + or − milliseconds, reflecting the lead or the lag time of the brain signal acquired from the first electrode compared with that of the second electrode, respectively.

As described in further detail in the Examples, the structure of the neuromarkers of the invention was originally designed based on coherence analysis as described above. However, rather than the 30-second epoch of standard sleep analyses, the coherence and phase delays for the present study were computed on a micro-level using a short duration of less than 5 seconds, guided by the underlying macro structure of the brain state belonging to one of the 5 sleep states: (1) Awake, (2) Stage I Sleep, (3) Stage II Sleep, (4) Delta-wave or stage III sleep, and (5) rapid-eye-movement (REM) sleep. The specific structure of the neuromarkers of the invention is based on a method of characterizing EEG time-frequency variations during awake and sleep state known in the art [34-39]. In preliminary studies, the coherences and phase analyses were applied to sleep records of PTSD patients and analysis was performed on pairs of EEG scalp positions from sleep studies of normal subjects and veterans with PTSD, and coherence and phase delays were calculated between specific intra-hemispheric pairs. Coherences and phase delays were computed for specific frequency bands of EEG pairs.

Systems for Detecting PTSD

In some embodiments, a system for detecting PTSD is provided. Such a system may comprise an EEG device for measuring brain wave function of a subject; a storage device; and a processor communicatively coupled to the storage device, wherein the processor executes application code instructions that are stored in the storage device and that cause the system to: obtain a brain wave pattern from the EEG device; determine a value for one or more of the neuromarkers described herein; and detect post-traumatic stress disorder in the subject by determining if the value of the one or more neuromarkers is above a designated threshold, or is increased or decreased relative to a control value.

A system as described herein may be used to obtain brain wave data or EEG data from a subject or patient during a sleep analysis, such as during a polysomnography study as described herein.

A system as described herein may incorporate all or part of the methods of the present invention. Such a system may further include an amplifier/transmitter unit, which may be a wireless unit, capable of measuring multiple channels of EEG in a highly dynamic environment and transmitting data to a commercial PC computer, and may further include at least one sensor. In some embodiments, an electrode wiring harness may be capable of handling multiple electrodes. In specific embodiments, such a system may employ the use of application codes and/or application code instructions that are stored in the storage device. Such components may further case the system to correlate the value of the one or more neuromarkers as described herein to data obtained from a diagnostic analysis tool. For example, an electro-oculogram and/or a chin electromyogram may be obtained and analyzed by a system as described herein.

In certain other embodiments, application code instructions stored in the storage device may further cause the system to correlate the value of the one or more neuromarkers to data obtained from a diagnostic criteria including, but not limited to, clinical history, mental status examination, duration of symptoms, clinician-administered symptom checklist, and patient self-report. In some embodiments, multiple such criteria may be used, or all of these. Data obtained from such criteria may be analyzed concurrently with data from a neuromarker of the invention. Such a system may incorporate data from multiple criteria or methods as described herein to calculate a score or numerical value as described herein, for diagnosing the presence or severity of PTSD in a subject or patient.

In still further embodiments, a system as described herein may have application codes or application code instructions that are stored in the storage device that further cause the system to correlate the value of the one or more neuromarkers as described herein to additional factors relating to the presence or absence of PTSD. For example, such a system may correlate data obtained from a neuromarker of the invention with, for example, data relating to persistent nightmares, severe nightmares, sleep disturbances, insomnia, poor daytime functioning, fatigue, mood disorders, and depression in a subject or patient.

In certain example embodiments, the components of the system may be incorporated into a single hand held or wearable device. The hand held or wearable device may further comprise communication hardware necessary to communicate the analysis obtained as described herein to a remote server or computer—such as at a clinic or doctor's office—using standard communication protocols such as cellular, wireless, and internet communication protocols.

Computer Program Product for Detecting PTSD

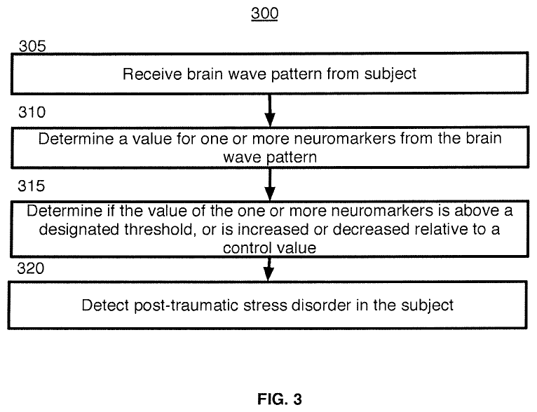

In certain embodiments, a computer program product is provided, comprising: a non-transitory computer-executable storage device having computer-readable instructions embodied thereon that when executed by a computer to detect PTSD in a subject or patient, the computer-executable program instructions comprising: computer-executable program instructions to receive a brain wave pattern; computer-executable program instructions to determine a value for one or more of the neuromarkers set forth herein from the brain wave pattern; and computer-executable programs instructions to detect post-traumatic stress disorder in the subject or patient by determining if the value of the one or more neuromarkers is above a designated threshold, or is increased or decreased relative to a control value. The steps employed by such a computer program product to detect PTSD in a subject are depicted in FIG. 3.

The example methods illustrated in FIG. 3 are described herein in further detail. These methods may also be performed with other systems and in other environments.

FIG. 3 is a block flow diagram depicting a method 300 to detect PTSD, in accordance with certain example embodiments.

Method 300 starts at block 305 where the computer-executable storage device receives a brain wave pattern. The brain wave pattern may be from a polysomnography (PSG) study as described herein, for example, and may be received remotely via electronic transmission.

At block 310, the computer-executable storage device analyzes the brain wave pattern received and determines a value for one or more neuromarkers according to the methods disclosed herein, and such as those set forth in Table 3, from the brain wave pattern. This brain wave pattern data and/or the value of the one or more neuromarkers may be stored within the computer-executable storage device.

At block 315, the computer-executable storage device determines whether the value of the one or more neuromarkers is above a designated threshold, or is increased or decreased relative to a control value. Such a determination may be performed by the computer-executable storage device by comparing the values for the neuromarkers from the test subject with control values. In accordance with the invention, a control value may be data from an individual or subject not having PTSD. A designated threshold may be determined relative to any individual control variable and may be specific for each subject. By executing the steps depicted as blocks 305-315, the computer-executable storage device detects PTSD in a subject.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing developments in computer programming, and the embodiments should not be construed as limited to any one set of computer program instructions. Further, a skilled programmer would be able to write such a computer program to implement an embodiment of the disclosed embodiments based on the present description. Therefore, disclosure of a particular set of program code instructions is not considered necessary for an adequate understanding of how to make and use embodiments of the invention. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

The example embodiments described herein can be used with computer hardware and software that perform the methods and processing functions described previously. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

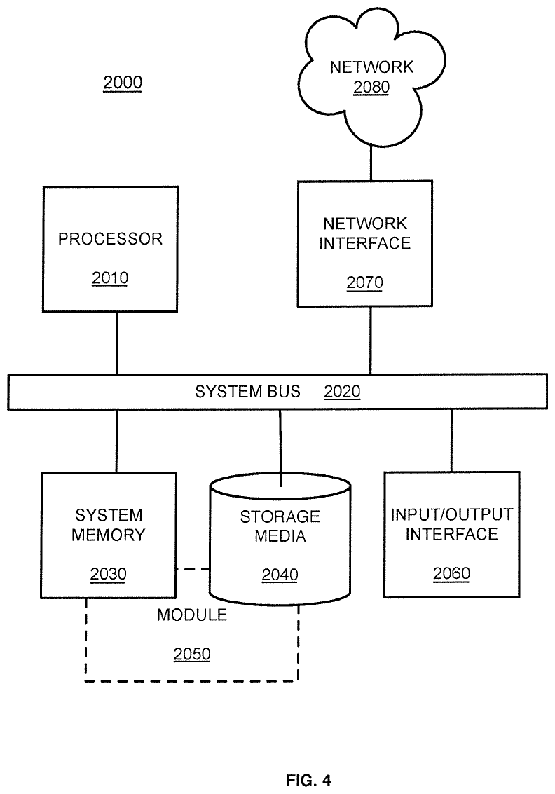

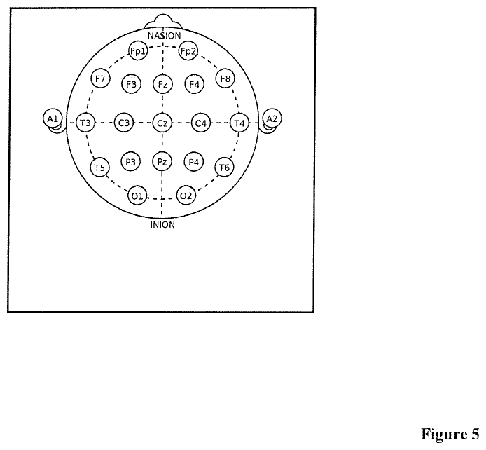

In example embodiments, the network computing devices and any other computing machines associated with the technology presented herein may be any type of computing machine such as, but not limited to, those discussed in more detail with respect to FIG. 4. Furthermore, any functions, applications, or modules associated with any of these computing machines, such as those described herein or any others (for example, scripts, web content, software, firmware, or hardware) associated with the technology presented herein may by any of the modules discussed in more detail with respect to FIG. 4. The computing machines discussed herein may communicate with one another, as well as with other computing machines or communication systems over one or more networks.

FIG. 4 depicts a computing machine 2000 and a module 2050 in accordance with certain example embodiments. The computing machine 2000 may correspond to any of the various computers, servers, mobile devices, embedded systems, or computing systems presented herein. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a wearable computer, a set-top box, a kiosk, a vehicular information system, one more processors associated with a television, a customized machine, any other hardware platform, or any combination or multiplicity thereof. The computing machine 2000 may be a distributed system configured to function using multiple computing machines interconnected via a data network or bus system.

The processor 2010 may be configured to execute code or instructions to perform the operations and functionality described herein, manage request flow and address mappings, and to perform calculations and generate commands. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 2010 may be a single processing unit, multiple processing units, a single processing core, multiple processing cores, special purpose processing cores, co-processors, or any combination thereof. According to certain embodiments, the processor 2010 along with other components of the computing machine 2000 may be a virtualized computing machine executing within one or more other computing machines.

The system memory 2030 may include non-volatile memories such as read-only memory (“ROM”), programmable read-only memory (“PROM”), erasable programmable read only memory (“EPROM”), flash memory, or any other device capable of storing program instructions or data with or without applied power. The system memory 2030 may also include volatile memories such as random access memory (“RAM”), static random access memory (“SRAM”), dynamic random access memory (“DRAM”), and synchronous dynamic random access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 may include, or operate in conjunction with, a non-volatile storage device such as the storage media 2040.

The storage media 2040 may include a hard disk, a floppy disk, a compact disc read-only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth.

The module 2050 may comprise one or more hardware or software elements configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for operably coupling the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCI”), PCI express (PCie), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including mice, touch-screens, scanners, electronic digitizers, sensors, receivers, touchpads, trackballs, cameras, microphones, keyboards, any other pointing devices, or any combinations thereof. The I/O interface 2060 may couple the computing machine 2000 to various output devices including video displays, speakers, printers, projectors, tactile feedback devices, automation control, robotic components, actuators, motors, fans, solenoids, valves, pumps, transmitters, signal emitters, lights, and so forth.

The computing machine 2000 may operate in a networked environment using logical connections through the network interface 2070 to one or more other systems or computing machines across the network 2080. The network 2080 may include wide area networks (WAN), local area networks (LAN), intranets, the Internet, wireless access networks, wired networks, mobile networks, telephone networks, optical networks, or combinations thereof. The network 2080 may be packet switched, circuit switched, of any topology, and may use any communication protocol. Communication links within the network 2080 may involve various digital or an analog communication media such as fiber optic cables, free-space optics, waveguides, electrical conductors, wireless links, antennas, radio-frequency communications, and so forth.

The processor 2010 may be connected to the other elements of the computing machine 2000 or the various peripherals discussed herein through the system bus 2020. It should be appreciated that the system bus 2020 may be within the processor 2010, outside the processor 2010, or both. According to some embodiments, any of the processor 2010, the other elements of the computing machine 2000, or the various peripherals discussed herein may be integrated into a single device such as a system on chip (“SOC”), system on package (“SOP”), or ASIC device.

In situations in which the systems discussed here collect personal information about users, or may make use of personal information, the users may be provided with an opportunity to control whether programs or features collect user information (e.g., information about a user's social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement an embodiment of the disclosed embodiments based on the appended flow charts and associated description in the application text. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

The example embodiments described herein can be used with computer hardware and software that perform the methods and processing functions described previously. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

The example systems, methods, and acts described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of various embodiments. Accordingly, such alternative embodiments are included in the inventions described herein.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

Although specific embodiments have been described above in detail, the description is merely for purposes of illustration. It should be appreciated, therefore, that many aspects described above are not intended as required or essential elements unless explicitly stated otherwise. For example, in alternative embodiments, certain acts may be performed in a different order, in parallel with one another, omitted entirely, and/or combined between different example embodiments, and/or certain additional acts can be performed, without departing from the scope and spirit of various embodiments. Accordingly, such alternative embodiments are included in the inventions described herein. Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

### Advantages of the Invention

The current practice for diagnosis and management of PTSD primarily relies on subjective assessments by the clinician in combination with patient self-report. An independent, objective and neurophysiology-based method for directly assessing brain function is desperately needed to improve diagnosis and management of PTSD. This need is highlighted by recommendations from the Institute of Medicine, National Academy of Science (IOM-NAS), which conducted a comprehensive assessment of the current PTSD diagnosis and treatment methods and identified potential shortcomings of the current diagnostic and treatment techniques (Treatment for Post-traumatic Stress Disorder in Military and Veteran Populations (2012), Institute of Medicine, National Academy of Science: ISBN 978-0-309-25421-2). A major recommendation by the IOM-NAS was to fill the urgent need for the development of methods for more precise and objective diagnosis of PTSD and its severity level, objective and faster evaluation of treatment efficacy, and ability to predict who might be at risk of relapse. The Neuromarkers described in this patent application addresses the critical need for developing objective methods for diagnosis of PTSD, determining its severity level, and potentially predicting treatment response early in therapy.

The invention is further described in the following examples, which do not limit the scope of the invention described in the claims.

### EXAMPLES

### Example 1—Structure of Neuromarkers

The structure of the neuromarkers used in the present study was originally designed based on coherence analysis as described above. However, coherence and phase delays were computed on a micro-level (short duration of <5 seconds) guided by the underlying macro structure of the brain state belonging to one of the 5 sleep states: (1) Awake, (2) Stage I Sleep, (3) Stage II Sleep, (4) Delta-wave or stage III sleep, and (5) rapid-eye-movement (REM) sleep.

In preliminary studies, these coherences and phase analyses were applied to sleep records of PTSD patients. The analysis was performed on the following pairs of EEG scalp positions from sleep studies of normal subjects and veterans with PTSD: Occipital Inter-hemispheric pair: 01 and 02, corresponding to the left and right hemisphere occipital lobe electrodes, respectively and according to the standard 10-20 EEG electrode placement guideline (Jasper, H. H. (1958). The ten twenty electrode system of the international federation. Electroenceph. and Clinical Neurophysiology, 10, 371-375). Central Inter-hemispheric: C3 and C4, corresponding to the central left and right hemisphere electrodes; Pre-frontal cortex inter-hemispheric pair: utilizing the eye-movement EOG electrodes that were placed directly above the left (1-EOG) and right eye (r-EOG), respectively. The coherence and phase delays were also computed between the following intra-hemispheric pairs: r EOG-C4; C4-O2; IEOG-C3; and C3-O1. The order of the pair was utilized, along with the sign of the corresponding phase angle, in order to determine the lead or lag times. For example, a positive phase angle between C4-02 corresponds to the right central site (C4) leading the right occipital region, while a negative phase delay points at the reverse (i.e., C4 lags 02 or, equivalently, 02 leads C4).

The coherences and phase delays were computed for specific frequency bands of EEG pairs. The analysis was performed on six distinct bands: Delta (8): 1-3.5 Hz; Theta (8): 4-7.5 Hz; Alpha (α): 8-12 Hz; Sigma (σ,): 13-16 Hz; Beta (β): 16.5-25 Hz; and Gamma (γ): 40 Hz. Furthermore, the dimension of analysis within each given sleep state and the awake state was also added, such that the coherences and phase angles were computed for 5 wake-sleep periods: Awake period with lights off and before falling asleep (W), 3 stages of non-REM sleep (Stages 1-3), and REM sleep.

### Example 2

The analysis was performed on the overnight PSG records of 2 groups: Norma, and PTSD. From each group, a total of 210 (7 EEG pairs×6 frequency bands×5 wake-sleep states) coherences and 210 phase angles were computed.

Normal Group—

The source of the data for this 7-subject cohort consisted of PSG records from a previous NIH supported grant (1R43HL076986-01A1, PI: Mo Modarres) where polysomnography (PSG) data (including EEG) were obtained from subjects with no sleep disorders, as well as patients with varying degrees of obstructive sleep apnea. The sleep evaluations were performed at night between 9:00 pm and 7:00 am according to standard clinical practices that included the attachment of 10 pairs of bio-potential and physiological surface electrodes/sensors such as EEG (electroencephalogram), EOG (electro-oculogram), EMG (electromyogram), ECG (electrocardiogram), as well as respiratory sensors (airflow) and pulse oximetry to the subject's body, face, and scalp. These studies were “attended” sleep studies, where a sleep technician continuously monitored the activity of the electrode signals in real time throughout the night (from an adjacent room to the patient's) to ensure signal integrity, and for the ability to reattach the sensors/electrodes in the event of loosening/detachment and/or the patient waking up to use the restroom. Following the conclusion of the study, standard sleep staging analysis was manually performed by a registered PSG technician, based on visual observation of the EEG, EOG, and chin EMG for each 30-second epoch duration, according to the standard clinical criteria originally developed by Rechtschaffen and Kales and updated by AASM [40, 41]. The PSG records and the corresponding sleep staging of the 7 normal subjects (with apnea-hypopnea index of <5 respiratory events/hour; considered clinically in the normal range) was considered normal.

PTSD Group—

The source of the data for the 7 normal subjects consisted of PSG records from veterans admitted to the James A Haley V A Poly-trauma Center in Tampa, Fla. These patients were referred to the hospital's sleep laboratory for a comprehensive sleep evaluation. Data acquisition and sleep staging of this group was based on standard clinical sleep medicine protocols, similar to the procedures described for the normal group above. IRB approval was obtained for the use of data from both groups for retrospective analysis of the PSG records. The two groups were age-matched such that the normal group had a mean age of 33.5 years (±8) and a range of 25-51 years old, and the PTSD group had a mean age of 33.5 (±9) and a range of 22-48 years of age.

### Example 3—Data Analysis and Results

For analysis of the macro-structure of sleep, the standard sleep architecture parameters that are typically reported in standard analysis for clinical/research sleep studies were computed. Table 15 shows a comparison of a number of sleep-architecture parameters obtained from the normal and PTSD groups. The two groups show virtually identical total sleep times and similar sleep efficiencies (% of time asleep while in bed). The total amount of time awake following sleep onset (WASO), as well as the fraction of sleep spent in stages 2 and 3, indicated in the table as “Stage 2(%)” and “Stage 3(%),” respectively, were also similar between the groups. In contrast, sleep latency (time from lights-out to the first episode of sleep), as well as the fraction of sleep spent in stages 1 and REM, each show statistical differences between the 2 groups. In particular, it appears that relative REM sleep duration is markedly decreased in PTSD, while the duration of Stage I is abnormally elevated, consistent with previous studies [25].

Similar to previous studies [26, 27], increased fragmentation in the sleep architecture was also observed. FIG. 1 shows examples of sleep architectures from a normal subject (top) and a PTSD patient (bottom). The vertical axis indicates the stage of sleep, where 1 corresponds to Stage 1, and 2 and 3 correspond to Stages 2 and 3, respectively. A value of 5 was chosen to represent REM sleep. From FIG. 1, sleep architecture in PTSD is more fragmented (i.e., less stable) than in normal sleep, and transition between sleep stages appears to be elongated for PTSD patients.

To further assess PTSD association with increased sleep variability, the average number of transitions (per hour of sleep) between all of the possible pairs of awake and sleep stages were computed. Thus, the transitions among 5 states of awake (W), Stage 1 (S 1), Stage 2 (S2), Stage 3 (S3), and REM produced a total of 20 paired transition types. T-test comparison was then performed for each of these transitions between the two groups.

TABLE 1
Comparison of PTSD and Normal Groups in Standard Sleep
Architecture Measures.

| | Normal (n = 7) | PTSD (n = 7) | |
|---|---|---|---|
| PSG Variable | Mean ± Std | Mean ± Std | P-value |
| Total Sleep Time (min) | 397.57 ± | 392.71 ± | 0.85 |
| Sleep Latency (min) | 16.02 ± 6.50 | 7.95 ± 3.78 | 0.015 |
| Wake after sleep onset | 33.14 ± 18.25 | 24.14 ± 24.97 | 0.46 |
| (WASO) (min) | | | |
| Sleep efficiency % | 88.86 ± 3.55 | 90.15 ± 7.03 | 0.67 |
| Stage 1(%) | 3 ± 2.16 | 10.61 ± 6.47 | 0.012 |
| Stage 2 (%) | 50.49 ± 6.26 | 52.73 ± 13.63 | 0.7 |
| Stage 3 (%) | 16.75 ± 5.78 | 17.22 ± 17.64 | 0.95 |
| REM (%) | 18.63 ± 3.4 | 10.68 ± 5.94 | 0.0097 |

Table 2 shows the results for the transition types that reached statistical significance, or were trending toward it. The PTSD and normal groups were comparable with the exception of the sleep latency, stage 1%, and REM % variables. Thus, from Table 16 it appears that PTSD is associated with excessive transitions between Stage 1 and Stage 2, as well as between REM and Stage 1. It can be noted that previous studies [26, 27] have reported similar increased transitions between REM and stage I in PTSD subjects. The present study, however, shows that there is also an increased transition between stages 1 and 2. The results of the standard macro-analysis of sleep structure is encouraging, in that it showed that the sleep architecture from the two study groups generally had characteristics and group differences that were consistent with previously reported studies, supporting the use of these data sets for computing and validating the novel neuromarkers described herein.

TABLE 2
Transitions between Sleep Stages for PTSD versus Normal Subjects.

| | Normal (n = 7) | PTSD (n = 7) | |
|---|---|---|---|
| | # of | # of | |
| Transition Type | transitions/hr | transitions/hr | P-value |
| S1 − + S2 | 1.38 ± 0.63 | 4.26 ± 2.93 | 0.03 |
| S1 − + REM | 0.17 ± 0.19 | 0.47 ± 0.40 | 0.09 |
| S2 − + S1 | 0.08 ± 0.11 | 2.93 ± 2.59 | 0.013 |
| REM − +S1 | 0.10 ± 0.13 | 0.59 ± 0.47 | 0.02 |

The next step of analysis was focused on computing the new neuromarkers based on the micro-analysis of EEG. The neuromarkers were inter- and intra-hemispheric coherences and phase delays computed from each subject's awake-sleep record: 14 Neuromarkers (7 Coherence and 7 Phase Delays)×5 states (Awake, Stages 1-3, REM)×6 frequency bands. Stepwise linear and logistic regressions were performed, using the neuromarkers as independent variables. Dependent variables were the two groups (normal and PTSD), as well as the standard clinical measure, the patient's PTSD checklist (PCL-C). PCL-C [42] is a 17-item self-report measure of the 17 symptoms of PTSD identified in the Diagnostic and Statistical Manual of Mental Disorders (DSM) [1]. The checklist evaluates the three-symptom clusters of PTSD: five re-experiencing symptoms, seven numbing/avoidance symptoms, and five hyper-arousal symptoms. PLC-C has a range of 17-85 (higher PCL-C values positively associated with more PTSD severity), where a value of above 50 is used as a cut-off for diagnosis of PTSD, and a minimum of a I O-point decrease is considered a clinically meaningful improvement in symptoms [43]. PCL reliability estimates range from 0.92 to 0.97, and the PCL has been validated in civilians and veterans [44-46].

Using a step-wise regress 10n analysis, 7 neuromarkers were identified that were significantly different in the PTSD group compared to the normal group, and that also exhibited a strong and significant association with PCL-C. This group of neuromarkers was all based on intra-hemispheric coherences and delays in the right hemisphere. The collective array of these seven identified neuromarkers is collectively referred to herein as PTSD neuromarkers.

Table 3 shows a comparison of the mean values of the PTSD neuromarkers between the normal and PTSD group. The right panel of Table 3 also shows the result of a Pearson's correlation analysis between each member of the PTSD neuromarkers group and the PCL-C severity rating for the PTSD group only. Thus, Table 3 reveals that PTSD neuromarkers are related to the intra-hemispheric coherence and phase delays on the right side of the brain, in alpha, beta, sigma, and gamma bands, and during stages 1, 2, and REM sleep. PTSD neuromarkers computed from the PTSD group show significant increases compared with normal subjects for the right intra-hemispheric coherences. Furthermore, PTSD neuromarkers appear to be highly and significantly correlated with PCL-C scores of PTSD patients, e.g., showing a very high correlation coefficient of 92% for right intra-hemispheric coherence of the gamma band during stage 2 sleep.

TABLE 3
Array of 7 PTSD Neuromarkers Showing Detection of Significant Differences
Between Normal and PTSD Groups, and Very Strong Correlations with PCL (PTSD group)

| | | | | Correlation Analysis | |
|---|---|---|---|---|---|
| | | | | between PTSD | |
| | Comparison | | | Neuromarkers and | |
| | between PTSD | | | PCL in | |
| | and Normal Group | | | PTSD Patients | |
| | Normal (n = 7) | PTSD | | Correlation | |
| PTSD Neuromarkers | Mean (Std) | (n = 7) | P-value | Coefficient | P-Value |
| Coherence: r EOG-C4 | 0.18 (0.05) | 0.72 (0.1) | 0.00001 | 0.78 | 0.035 |
| Stage 1; Alpha (8-12 Hz) | | | | | |
| Coherence: r EOG-C4 | 0.16 (0.07) | 0.69 (0.14) | 0.00001 | 0.79 | 0.034 |
| Stage 1; Sigma (13- | | | | | |
| Coherence: r EOG-C4 | 0.15 (0.05) | 0.62 (0.19) | 0.00001 | 0.83 | 0.022 |
| Stage 1; Beta (16- | | | | | |
| Coherence: r EOG-C4 | 0.16 (0.05) | 0.60 (0.19) | 0.003 | 0.90 | 0.005 |
| Stage 1; Gamma | | | | | |
| (35-45Hz) | | | | | |
| Coherence: r EOG-C4 | 0.20 (0.02) | 0.60 (0.19) | 0.0002 | 0.83 | 0.021 |
| Stage 2; Sigma (13- | | | | | |
| Coherence: r EOG-C4 | 0.13 (0.02) | 0.51 (0.29) | 0.005 | 0.92 | 0.004 |
| Stage 2; Gamma | | | | | |
| (35-45 Hz) | | | | | |
| Phase: C4-02 | −0.05 (0.22) | 0.25 (0.24) | 0.036 | 0.77 | 0.04 |
| REM; Sigma (13-16 Hz) | | | | | |

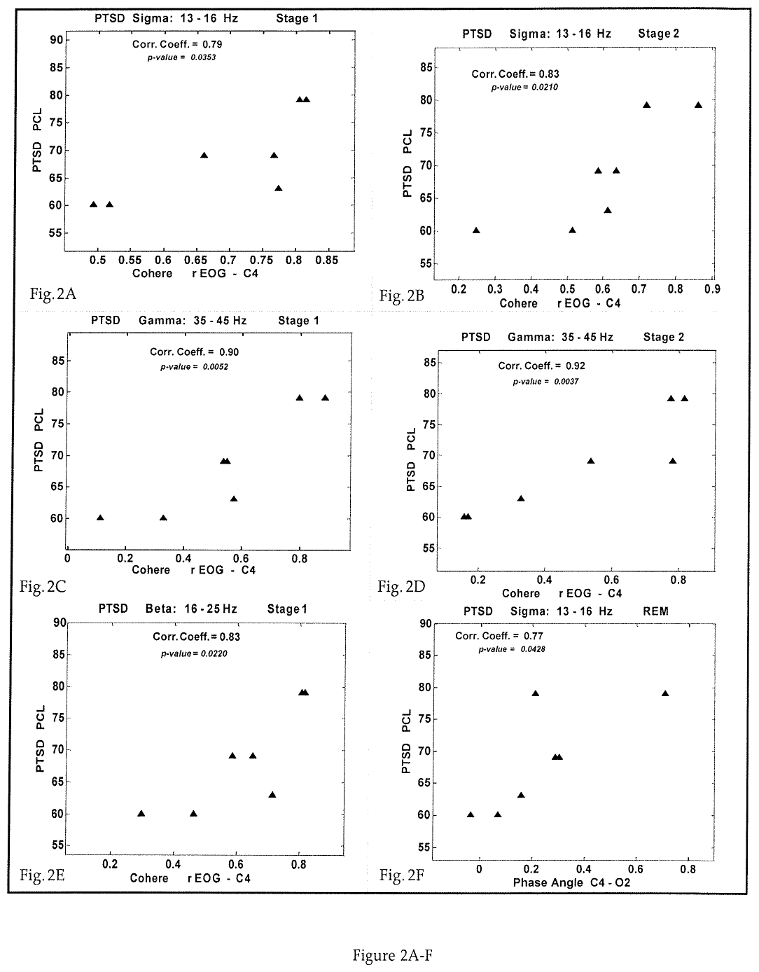

Finally, for a visualization of the degree of association and correlation of PTSD neuromarkers with PCL-C, FIG. 2 shows the correlation plots (Pearson correlation) of six PTSD neuromarkers (computed from the PTSD patient data). These results are very encouraging and strongly suggest the potential of the PTSD neuromarkers for diagnosis and quantification of the severity of PTSD, and objective assessment of treatment outcome.

Various modifications and variations of the described methods and systems of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

### Example 4

Materials and Method

PTSD neuromarkers are computed based on the levels of spectral power coherence and synchronicity between pairs of scalp EEG activity. Coherence is a normalized quantity (index spans from 0 to 1) that reflects the degree of association or coupling of the spectral power levels in a pair of EEG waveforms (from two scalp sites) and for a given frequency band. Formally, coherence is a generalization of correlation analysis and is computed as the magnitude of normalized cross-power spectrum of a pair of simultaneously recorded EEGs from two separate scalp locations.

$${C}_{xy} ( f ) = \frac{{\vert {G}_{xy} ( f ) \vert}^{2}}{{G}_{xx} ( f ) {G}_{yy} ( f )} \qquad Eq . 1$$

Denoting EEG signals recorded at two scalp locations as x and y, coherence between x and y is defined as Eq. 1, where G_{xy}(t) is the cross-spectral density between x and y at the frequency f, and Gₓₓ(f) and G_{yy}(f) are the auto-spectral density (at frequency f) of x and y, respectively. Such EEG coherences reflect the degree of coupling and functional association between two brain regions and can be computed for any specific frequency bands of EEG pairs.

Source of Data: sleep EEG records from a small cohort (n=38) of Veterans diagnosed with PTSD, and a control group of 38 Veterans without PTSD, who had all undergone clinical sleep studies at the VA Portland Sleep Laboratory. Standard measurement of PTSD symptoms using the PTSD checklist (PCL-5), were also obtained at the time of the subjects' sleep study. The PCL-5 [47] is a 20-item self-report which assesses the 20 PTSD symptoms outlined in the Diagnostic and Statistical Manual of Mental Disorders, 5^{th} Edition (DSM-V) [48]. PCL-5 total symptom severity score ranges from 0 (no reported symptoms) to a maximum of 80, and a value of 33 is estimated as a diagnostic threshold for PTSD. FIG. 5 shows the standard clinical sleep EEG locations according the International 10-20 EEG placement sites (Jasper, H. H. (1958). The ten twenty electrode system of the international federation. Electroenceph. and Clinical Neurophysiology, 10, 371-375). The EEG Montage for the sleep study consisted of the following leads: Frontal: F3 and F4; Central: C3 and C4, Occipital O1 and O2, all are measured with respect to the two reference signals measured at A1 and A2.

Data Analysis: 15 coherence values were computed for all of the electrode pairs: O1-O2; C3-C4; F3-F4; O1-C3; O2-C4; C3-F3; C4-F4; O1-C4; O2-C3; C3-F4; C4-F3; O1-F3; O1-F4; O2-F3; and O2-F4, where all of the EEG signals at each location are measured with respect to the two reference signals measured at A1 and A2.

The coherence values were computed for the entire sleep study period using 5-second sliding windows that were overlapped by 1 second for frequencies from 0.2 Hz to 50 Hz and 0.2 Hz resolution (i.e., frequencies were 0.2, 0.4, 0.6, 0.8, 1.0, 1.2, . . . 50 Hz). Independently, the sleep records were scored for various states of sleep according to standard clinical sleep staging. Thus, each 30-second epoch of data was scored as one of the following states: (1) Awake, (2) Stage I Sleep, (3) Stage II Sleep, (3) Delta-wave or stage III sleep, and (5) rapid-eye-movement (REM) sleep.

The structure of our neuromarkers was originally designed based on the coherence analysis. However, the coherence and phase delays were computed on a micro-level (short duration of <5 seconds) guided by the underlying macro structure of the brain state belonging to one of the 5 sleep states:

1. Awake period with lights off and before falling asleep (W); 2. Stage I Sleep; 3.Stage II Sleep; 4. Delta-wave or stage III sleep; 5. Rapid-eye-movement (REM) sleep.

Analysis and Discovery of PTSD Sensitive Neuromarkers

In the first stage of analysis, median coherence values for each of the 5 awake/sleep states were computed for all of the 15 electrode pairs and all frequencies (250 frequency values corresponding to 0.2, 0.4, . . . , 50 Hz).

In the second stage of the analysis, and for each sleep state, we computed all of the possible ratios of coherences among the 15 electrode pairs (105 ratios) pairs and for each of the 250 frequency values, referred to as markers. We then performed step-wise linear and logistic regressions, with the ratios as independent variables while belonging to groups (control or PTSD) were used as the dependent variables.

The candidate neuromarkers were then computed based on linear combination of the particular markers that produced the most significant discrimination among the two groups based on the R² and F statistics. In addition, a candidate neuromarker may be a single marker, taking into account that such single marker serving as a neuromarker may have a lower specificity with potentially greater false positive or negative in detecting PTSD, or alternatively such single marker may be less predictive of PTSD symptom severity, than neuromarkers obtained from combination of two or more markers.

Results

Our analysis showed that the neuromarkers computed during awake and stage 2 sleep produced the most significant separation between control and PTSD group.

Awake State:

1. Neuromarkers for Diagnosis of PTSD

We define “PTSD_Diag_Wake” as one Neuromarker that distinguished PTSD from control from data obtained during awake state. This particular Neuromarker is computed from a combination of the below 6 markers using multiple linear regression, shown in Table 4 below. Table 4 bottom contains the intercept and slope coefficients for each of the 6 markers used in the multiple linear regression:

TABLE 4

| Marker_1 | Coh. O1—O2 @ 0.6 Hz.)/Coh. O1—F4 @ 2.4 Hz) |
|---|---|
| Marker_2 | Coh. C3—C4 @ 23.8 Hz)/Coh. O2—C3 @ 37.0 Hz) |
| Marker_3 | Coh. F3—F4 @ 8.6 Hz)/Coh. C3—F3 @ 6.8 Hz) |
| Marker_4 | Coh. F3—F4 @ 7.4 Hz)/Coh. C3—F4 @ 7.6 Hz) |
| Marker_5 | Coh. F3—F4 @16.8 Hz)/Coh. O1—F4 @ 7.4 Hz) |
| Marker_6 | Coh. O1—F4 @ 4.2 Hz)/Coh. O2—F3 @ 13.0 Hz) |
| Intercept | 1.92 |
| Slope for Marker_1 | 0.11 |
| Slope for Marker_2 | −0.27 |
| Slope for Marker_3 | 0.72 |
| Slope for Marker_4 | 0.07 |
| Slope for Marker_5 | 0.11 |
| Slope for Marker_6 | 0.31 |

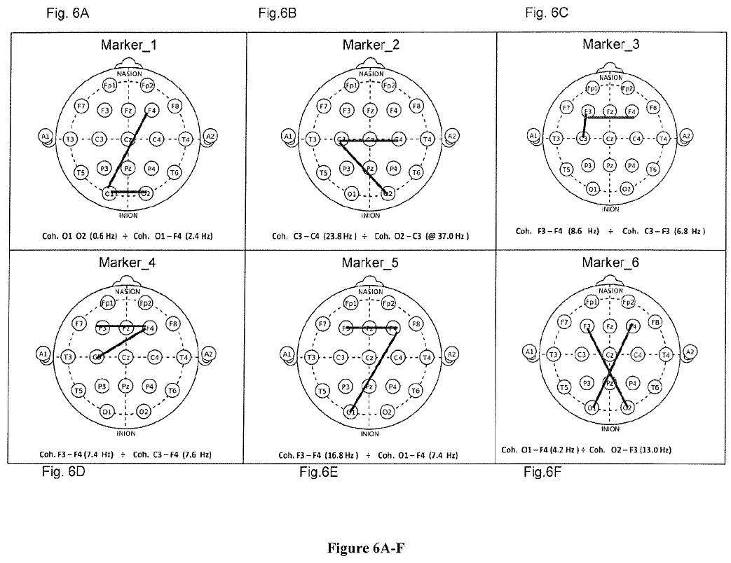

FIG. 6a-f shows graphically the 6 markers that are included in the PTSD_Diag_Wake Neuromarker.
Table 5 below shows the statistical comparison of PTSD_Diag_Wake between the two groups:

TABLE 5

| | Control | PTSD | Regression Statistics: | | |
|---|---|---|---|---|---|
| PTSD_Diag_Wake | (n = 38) | (n = 38) | R2 | F value | p value |
| Mean (Std) | 1.11 (0.18) | 1.90 (0.23) | 0.79 | 42.1 | 0.0000 |
| Median | 1.12 | 1.88 | | | |

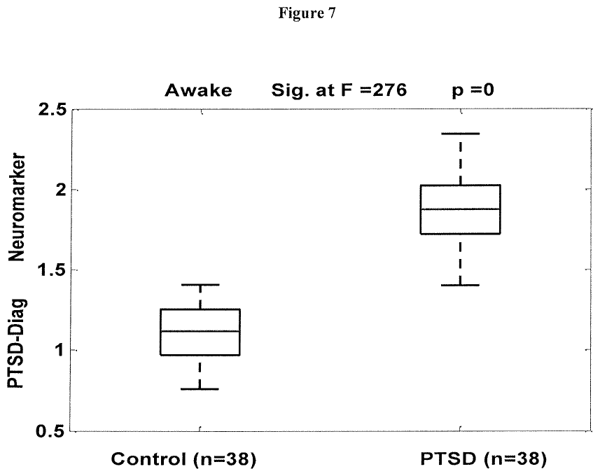

ANOVA analysis between the PTSD_Diag_Wake is shown herein. In FIG. 7, PTSD_Diag_Wake values of the two groups are shown in boxplot format, the top and bottom lines of boxes representing the 75^{th} and 25^{th} percentiles, median value shown as a red line, and the whiskers depict the range. Based on the regression and ANOVA analysis, values of 1.5 and above for PTSD_Diag_Wake Neuromarker appear to correspond to the presence of PTSD (FIG. 7).

1. Neuromarkers Associated with Severity of PTSD

PTSD_Symptom_Wake is a Neuromarker that is sensitive to severity of PTSD symptoms as determined by the PCL-5 scale (patient reported). It is computed as the ratio of
Coh. C3-C4 (@ 20.0 Hz.)/Coh. O2-C3 (@ 6.8 Hz)

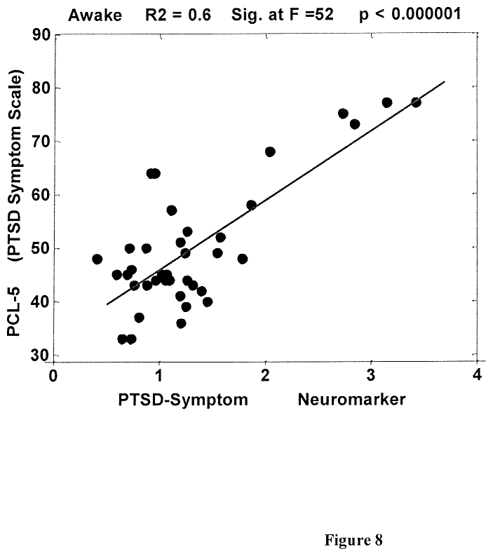

Computed from awake state before sleep initiation. FIG. 8 shows the scatter plot and regression line between PCL-5 and PTSD_Symptom_Wake Neuromarker.

Improved PTSD-Symptom-Wake Based on Combination of Markers

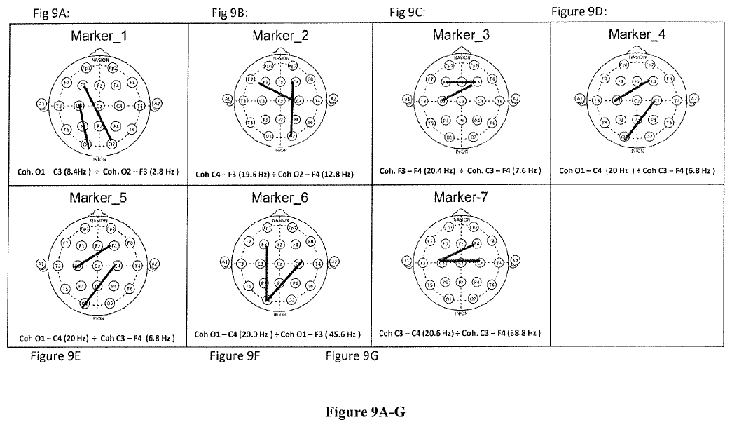

This particular Neuromarker is computed from a combination of the 7 markers (FIG. 9A-G) using multiple linear regression, shown in Table 6 below. Table 6 bottom contains the intercept and slope coefficients for each of the 7 markers used in the multiple linear regression. For these analysis, the dependent variable was PCL-5, and the independent variable was PTSD-Symptom-W based on combination of the most sensitive markers to PTSD:

TABLE 6

| Marker_1 | Coh. O1-C3 (@ 8.4 Hz)/Coh. O2-F3 (@ 2.8 Hz) |
|---|---|
| Marker_2 | Coh. C4-F3 (@ 19.6 Hz)/Coh. O2-F4 (@ 12.8 Hz) |
| Marker_3 | Coh. F3-F4 (@ 20.4 Hz)/Coh. C3-F4 (@ 7.6 Hz) |
| Marker_4 | Coh. O1-C4 (@ 20 Hz)/Coh. C3-F4 (@ 6.8 Hz) |
| Marker_5 | Coh. C3-F3 (@ 0.2 Hz)/Coh. O1-F3 (@ 6.8 Hz) |
| Marker_6 | Coh. O1-C4 (@ 20.0 Hz)/Coh. O1-F3 (@ 45.6 Hz) |
| Marker_7 | Coh. C3-C4 (@ 20.6 Hz)/Coh. C3-F4 (@ 38.8 Hz) |
| Intercept | −6.62 |
| Slope for Marker_1 | 4.96 |
| Slope for Marker_2 | 6.97 |
| Slope for Marker_3 | 9.93 |
| Slope for Marker_4 | 5.08 |
| Slope for Marker_5 | 22.89 |
| Slope for Marker_6 | −10.65 |
| Slope for Marker_7 | 32.96 |

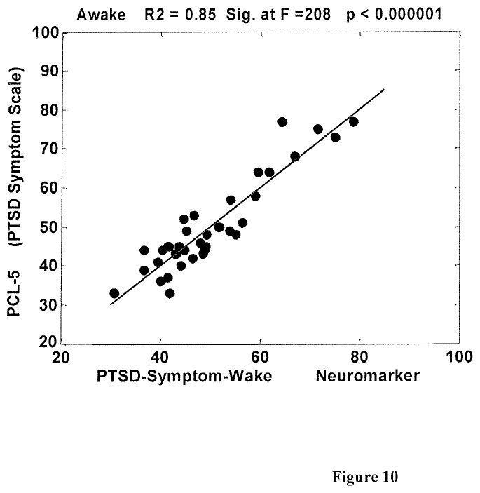

FIG. 9A-G show graphically the seven markers that are included in the PTSD_Symptom_Wake Neuromarker. For each marker, the figure shows the two pairs of EEG electrodes whose coherence were utilized to calculate the maker. For example, in FIG. 9-A, Marker_1 is computed from the coherence between O1-C3 pair at 8.4 Hz divided by the coherence between O2-F3 computed at 2.8 Hz. The scatter plot and regression line of PTSD_Symptom_Wake vs PCL-5 is shown in FIG. 10. The PTSD_Symptom_Wake was computed based on the combinations of the seven markers shown in FIG. 9. The relationship between the Neuromarker and PCL-5 was strong and significant with an R² of 0.85 (F=208, p<10⁻⁶).

### Repeat Awake Using a 1 Hz Frequency Bands

We use the single frequencies that we obtained from the analysis as a starting point to obtain additional makers based on frequency bands rather than single frequencies. Furthermore, we used new markers based on a frequency band (for example, 1 second window around a single frequency for a 1 Hz frequency band width) as an anchor and analyzed other markers based on a frequency band rather than a single frequency. We were able to obtain neuromarkers based on a range of frequencies rather than single frequencies.

Neuromarkers for Diagnosis of PTSD

In this example, we define “PTSD_Diag_Wake” as one Neuromarker that distinguished PTSD from control from data obtained during awake state. This particular Neuromarker is computed from a combination of the below 2 markers using multiple linear regression, shown in the Table 7 below. Table 7 bottom in the right contains the intercept and slope coefficients for each of the 2 markers used in the multiple linear regression:

TABLE 7

| Marker_1 | Coh. F3-F4 (8.6-9.6 Hz.)/Coh. C3-F3 (@ 6.8-7.8 Hz) |
|---|---|
| Marker_2 | Coh. F3-F4 (@ 7.6-8.6 Hz)/Coh. O2-F3 (@ 7.4-8.4 Hz) |
| Intercept | 1.16 |
| Slope for Marker_1 | 0.92 |
| Slope for Marker_2 | −0.27 |

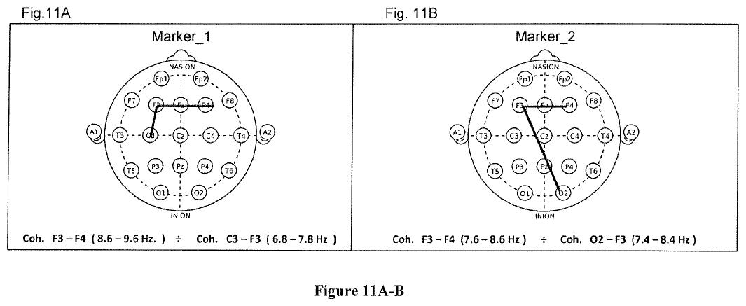

FIG. 11a-b shows herein graphically the 2 markers that are included in the PTSD_Diag_Wake Neuromarker.

Table 8 below shows the statistical comparison of PTSD_Diag_Wake between the two groups:

TABLE 8

| | Control | PTSD | Regression Statistics: | | |
|---|---|---|---|---|---|
| PTSD_Diag_Wake | (n = 38) | (n = 38) | R² | F value | p value |
| Mean (Std) | 1.08 (0.12) | 1.92 (0.24) | 0.83 | 181.5 | 0.0000 |
| Median | 1.07 | 1.83 | | | |

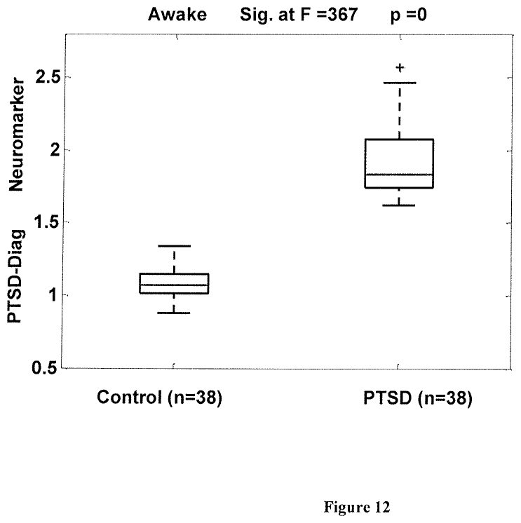

ANOVA analysis between the PTSD_Diag_Wake is shown herein. In FIG. 12 PTSD_Diag_Wake values of the two groups are shown in boxplot format, the top and bottom lines of boxes representing the 75^{th} and 25^{th} percentiles, median value shown as a red line, and the whiskers depict the range. Based on the regression and ANOVA analysis, values of 1.5 and above for PTSD_Diag_Wake Neuromarker appear to correspond to the presence of PTSD.

Neuromarkers Associated with Severity of PTSD Using 1 Hz Band

### PTSD-Symptom-Awake Based on Combination of Markers

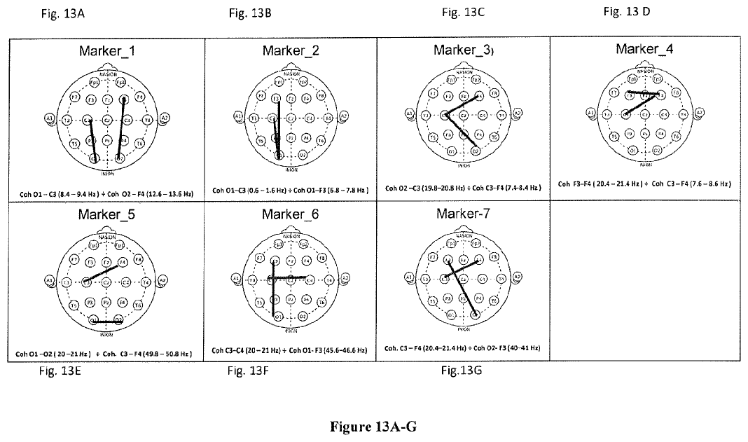

This particular Neuromarker is computed from a combination of the 7 markers (FIG. 13A-G) using multiple linear regression, shown in Table 9 below. Table 9 bottom contains the intercept and slope coefficients for each of the 7 markers used in the multiple linear regression. For these analysis, the dependent variable was PCL-5 (patient symptoms), and the independent variable was PTSD-Symptom-W based on combination of the most sensitive markers to PTSD obtained during awake period.

TABLE 9

| Marker_1 | Coh. O1-C3 (8.4-9.4 Hz)/Coh. O2-F4 (12.6-13.6 Hz) |
|---|---|
| Marker_2 | Coh. O1-C3 (0.6-1.6 Hz)/Coh. O1-F3 (6.8-7.8 Hz) |
| Marker_3 | Coh. O2-C3 (19.8-20.8 Hz)/Coh. C3-F4 (7.4-8.4 Hz) |
| Marker_4 | Coh. F3-F4 (20.4-21.4 Hz)/Coh. C3-F4 (7.6-8.6 Hz) |
| Marker_5 | Coh. O1-O2 (20-21 Hz)/Coh. C3-F4 (49.8-50.8 Hz) |
| Marker_6 | Coh. C3-C4 (20-21 Hz)/Coh. O1-F3 (45.6-46.6 Hz) |
| Marker_7 | Coh. C3-F4 (20.4-21.4 Hz)/Coh. O2-F3 (40-41 Hz) |
| Intercept | 1.04 |
| Slope for Marker_1 | 9.18 |
| Slope for Marker_2 | 9.96 |
| Slope for Marker_3 | 7.67 |
| Slope for Marker_4 | 10.98 |
| Slope for Marker_5 | −21.75 |
| Slope for Marker_6 | 19.66 |
| Slope for Marker_7 | 16.27 |

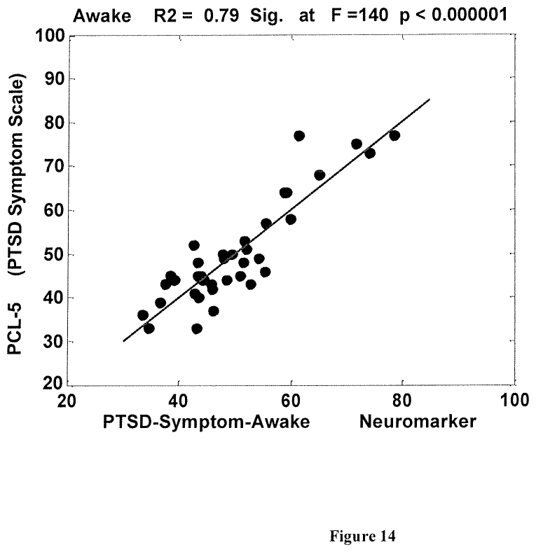

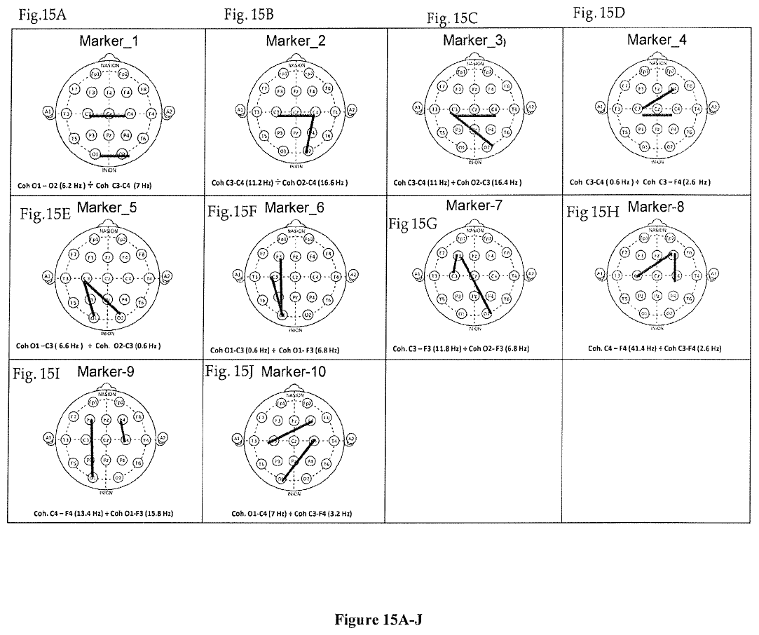

FIG. 13 A-G show graphically the seven markers that are included in the PTSD_Symptom_Wake Neuromarker based on coherence calculation using a 1-Hz frequency band-width. For each marker, the figure shows the two pairs of EEG electrodes whose coherence were utilized to calculate the maker. For example, in FIG. 13-A, Marker_1 is computed from the coherence between O1-C3 pair in the 8.4-9.4 Hz frequency band, divided by the coherence between O2-F4 computed in the 12.6-13.6 Hz band. A scatter plot and regression line of PTSD_Symptom_Wake (based on the seven markers using 1-Hz frequency band-width) vs PCL-5 is shown in FIG. 14. The relationship between the Neuromarker and PCL-5 was strong and significant with an R² of 0.79 (F=140, p<10⁻⁶).

### II. Stage 2 Sleep:

### (1) Neuromarkers for Diagnosis of PTSD

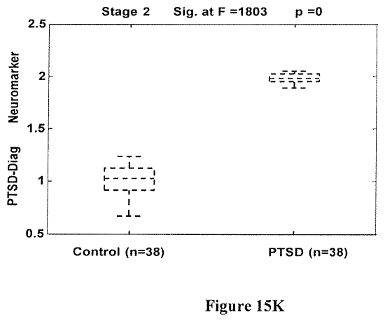

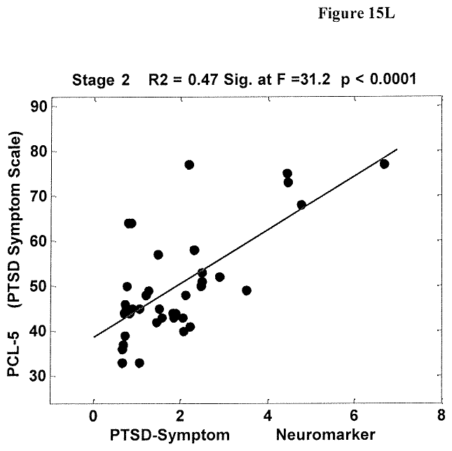

We define “PTSD_Diag_Stage2” as another Neuromarker that distinguished PTSD from control from data obtained during Stage 2 sleep. This particular Neuromarker is computed from a combination of the 10 markers (FIG. 15A-J) using multiple linear regression, shown in Table 10 below. Table 10 bottom contains the intercept and slope coefficients for each of the 10 markers used in the multiple linear regression.

TABLE 10

| Marker_1 | Coh. O1-O2 (@ 6.2 Hz.)/Coh. C3-C4 (@ 7.0 Hz) |
|---|---|
| Marker_2 | Coh. C3-C4 (@ 11.2 Hz)/Coh. O2-C4 (@ 16.6 Hz) |
| Marker_3 | Coh. C3-C4 (@ 11.0 Hz)/Coh. O2-C3 (@ 16.4 Hz) |
| Marker_4 | Coh. C3-C4 (@ 0.6 Hz)/Coh. C3-F4 (@ 2.6 Hz) |
| Marker_5 | Coh. O1-C3 (@ 6.6 Hz)/Coh. O2-C3 (@ 0.6 Hz) |
| Marker_6 | Coh. O1-C3 (@ 0.6 Hz)/Coh. O1-F3 (@ 6.8 Hz) |
| Marker_7 | Coh. C3-F3 (@ 11.8 Hz)/Coh. O2-F3 (@ 6.8 Hz) |
| Marker_8 | Coh. C4-F4 (@ 41.4 Hz)/Coh. C3-F4 (@ 2.6 Hz) |
| Marker_9 | Coh. C4-F4 (@ 13.4 Hz)/Coh. O1-F3 (@ 15.8 Hz) |
| Marker_10 | Coh. O1-C4 (@ 7.0 Hz)/Coh. C3-F4 (@ 3.2 Hz) |
| Intercept | 2.71 |
| Slope for Marker_1 | 0.16 |
| Slope for Marker_2 | 0.17 |
| Slope for Marker_3 | 0.17 |
| Slope for Marker_4 | 0.07 |
| Slope for Marker_5 | 0.09 |
| Slope for Marker_6 | 0.08 |
| Slope for Marker_7 | 0.05 |
| Slope for Marker_8 | 0.09 |
| Slope for Marker_9 | 0.10 |
| Slope for Marker_10 | 0.07 |

Table 11 below shows the statistical comparison of PTSD_Diag_Stage2 between the two groups:

| | Control | PTSD | Regression Statistics: | | |
|---|---|---|---|---|---|
| PTSD_Diag_Stage2 | (n = 38) | (n = 38) | R² | F value | P value |
| Mean (Std) | 1.02 (0.13) | 1.98 (0.05) | 0.96 | 158.38 | 0.00 |
| Median | 1.03 | 1.98 | | | |

ANOVA analysis between the PTSD_Diag_Stage2 is shown herein. In FIG. 15K, PTSD_Diag_Stage2 values of the two groups are shown in boxplot format, the top and bottom lines of boxes representing the 75^{th} and 25^{th} percentiles, median value shown as a red line, and the whiskers depict the range. Based on the regression and ANOVA analysis, values of 1.75 and above for PTSD_Diag_Stage2 Neuromarker appear to correspond to the presence of PTSD.
(1) Neuromarkers Associated with Severity of PTSD
PTSD_Symptom_Stage 2 is a Neuromarker that is sensitive to severity of PTSD symptoms as determined by the PCL-5 scale (patient reported). It is computed as the ratio of
Coh. C3-F4 (@ 19.6 Hz.)/Coh. O2-F3 (@ 10.0 Hz)
   Computed from stage 2 sleep. FIG. 15L shows the scatter plot and regression line between PCL-5 and PTSD_Symptom_Stage 2 Neuromarker.

### Improved PTSD-Symptom-S2 Based on Combination of Markers

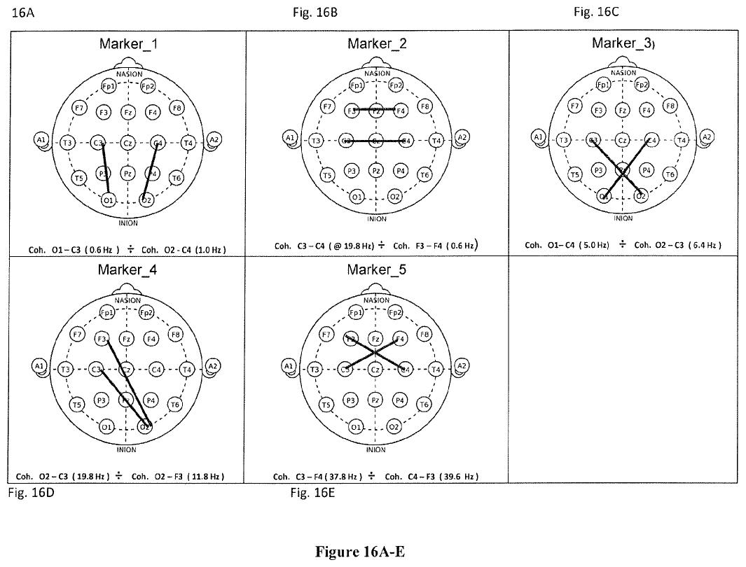

This particular Neuromarker is computed from a combination of the 5 markers (FIG. 16A-E) using multiple linear regression, shown in Table 12 below. Table 12 bottom contains the intercept and slope coefficients for each of the 5 markers used in the multiple linear regression. For these analysis, the dependent variable was PCL-5 (patient symptoms), and the independent variable was PTSD_Symptom-S2 based on combination of the most sensitive markers to PTSD obtained during stage II sleep period:

TABLE 12

| Marker_1 | Coh. O1-C3 (@ 0.6 Hz)/Coh. O2-C4 (@ 1.0 Hz) |
|---|---|
| Marker_2 | Coh. C3-C4 (@ 19.8 Hz)/Coh. F3-F4 (@ 0.6 Hz) |
| Marker_3 | Coh. O1-C4 (@ 5.0 Hz)/Coh. O2-C3 (@ 6.4 Hz) |
| Marker_4 | Coh. O2-C3 (@ 19.8 Hz)/Coh. O2-F3 (@ 11.8 Hz) |
| Marker_5 | Coh. C3-F4 (@ 37.8 Hz)/Coh. C4-F3 (@ 39.6 Hz) |
| Intercept | −125.09 |
| Slope for Marker_1 | 43.40 |
| Slope for Marker_2 | 6.95 |
| Slope for Marker_3 | 42.77 |
| Slope for Marker_4 | 7.52 |
| Slope for Marker_5 | 82.32 |

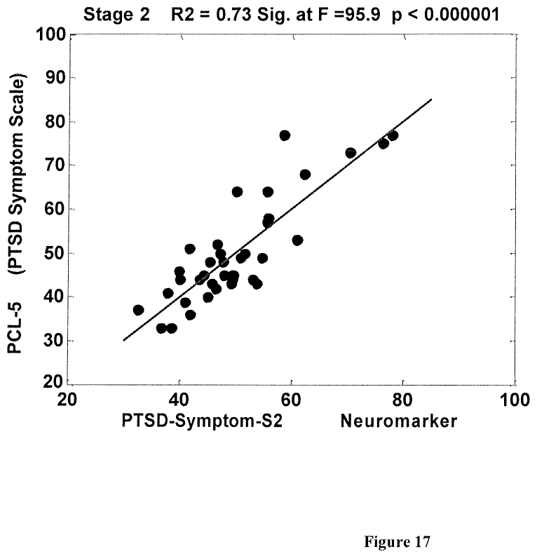

FIG. 16A-E show graphically the 5 markers that are included in the PTSD_Symptom_S2 Neuromarker computed during Stage 2 sleep. For each marker, the figure shows the two pair of EEG electrodes whose coherence were utilized to calculate the maker. For example, in FIG. 16-A, Marker_1 is computed from the coherence between 01-C3 pair at 0.6 Hz divided by the coherence between O2-C4 computed at 1.0 Hz. A scatter plot and regression line of PTSD_Symptom S2 vs PCL-5 are shown in FIG. 17. This PTSD_Symptom_S2 is computed based on the combinations of the five markers shown in FIG. 16. The relationship between the Neuromarker and PCL-5 is strong and significant with an R² of 0.73 (F=96 p<10⁻⁶).

### Repeat Stage 2 Using a 1 Hz Frequency Bands

We used the frequencies that we obtained from the analysis as a starting point to obtain additional makers based on frequency bands rather than single frequencies. Furthermore, we used new markers based on a frequency band (for example, 1 second window around a single frequency for a 1 Hz frequency band) as an anchor and analyzed other markers based on a frequency band rather than a single frequency. We were able to obtain neuromarkers based on a range of frequencies rather than single frequencies.

### Neuromarkers for Diagnosis of PTSD

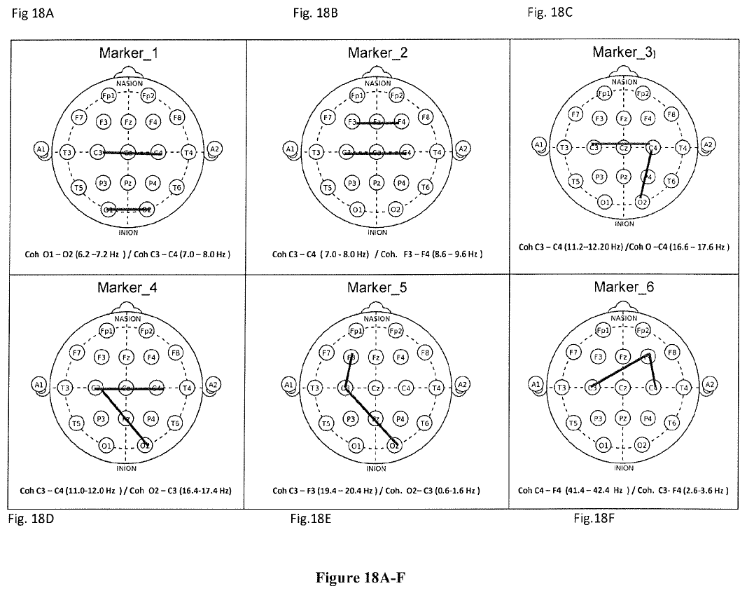

We define “PTSD_Diag_Stage2” as another Neuromarker that distinguished PTSD from control from data obtained during Stage 2 sleep. This particular Neuromarker is computed from a combination of the 6 markers (FIG. 18A-F) using multiple linear regression, shown in Table 13 below. Table 13 bottom contains the intercept and slope coefficients for each of the 6 markers used in the multiple linear regression.

TABLE 13

| Marker_1 | Coh. O1-O2 (6.2-7.2 Hz)/Coh. C3-C4 (7.0-8.0 Hz) |
|---|---|
| Marker_2 | Coh. C3-C4 (7.0-8.0 Hz)/Coh. F3-F4 (8.6-9.6 Hz) |
| Marker_3 | Coh. C3-C4 (11.2-12.20 Hz)/Coh. O2-C4 (16.6-17.6 Hz) |
| Marker_4 | Coh. C3-C4 (11.0-12.0 Hz)/Coh. O2-C3 (16.4-17.4 Hz) |
| Marker_5 | Coh. C3-F3 (19.4-20.4 Hz)/Coh. O2-C3 (0.6-1.6 Hz) |
| Marker_6 | Coh. C4-F4 (41.4-42.4 Hz)/Coh. C3-F4 (2.6-3.6 Hz) |
| Intercept | 2.40 |
| Slope for Marker_1 | 0.17 |
| Slope for Marker_2 | 0.02 |
| Slope for Marker_3 | 0.2305 |
| Slope for Marker_4 | 0.25 |
| Slope for Marker_5 | 0.09 |
| Slope for Marker_6 | 0.12 5 |

Table 14 below shows the statistical comparison of PTSD_Diag_Stage2 between the two groups:

TABLE 14

| | | | Regression Statistics: | | |
|---|---|---|---|---|---|
| | Control | PTSD | | F | p |
| PTSD_Diag_Stage2 | (n = 38) | (n = 38) | R² | value | value |
| Mean (Std) | 1.04 (0.19) | 1.96 (0.03) | 0.93 | 143.75 | 0.00 |
| Median | 1.02 | 1.96 | | | |

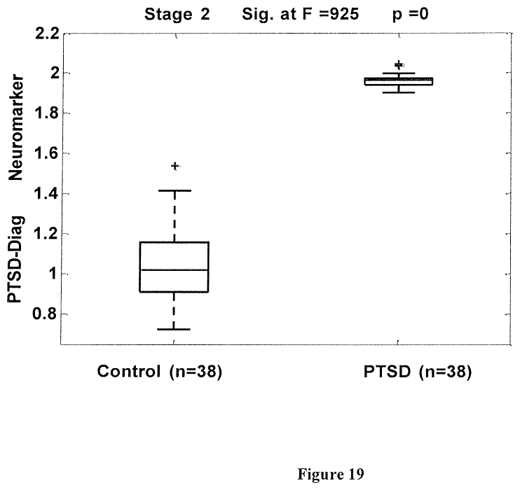

ANOVA analysis between the PTSD_Diag_Stage2 is shown herein. In FIG. 19 PTSD_Diag_Stage2 values of the two groups are shown in boxplot format, the top and bottom lines of boxes representing the 75^{th} and 25^{th} percentiles, median value shown as a red line, and the whiskers depict the range. Based on the regression and ANOVA analysis, values of 1.8 and above for PTSD_Diag_Stage2 Neuromarker appear to correspond to the presence of PTSD.

Neuromarkers Associated with Severity of PTSD Using 1 Hz Band

### PTSD-Symptom-S2 Based on Combination of Markers

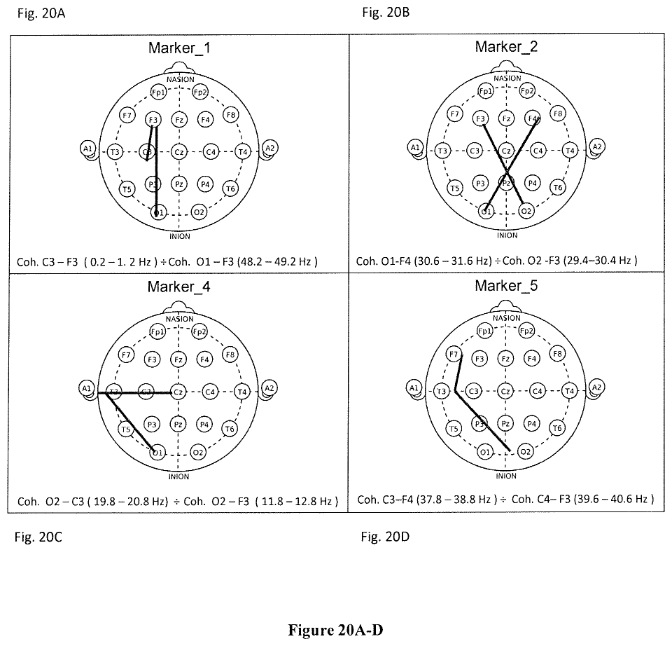

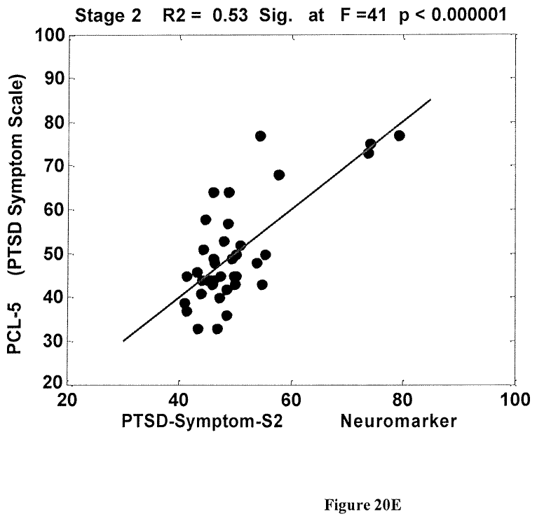

This particular Neuromarker is computed from a combination of the 4 markers (FIG. 20A-D) using multiple linear regression, shown in the Table 15 below. Table 15 bottom contains the intercept and slope coefficients for each of the 4 markers used in the multiple linear regression. For these analysis, the dependent variable was PCL-5 (patient symptoms), and the independent variable was PTSD-Symptom-S2 based on combination of the most sensitive markers to PTSD:

TABLE 15

| Marker_1 | Coh. C3-F3 (0.2-1.2 Hz)/Coh. O1-F3 (48.2-49.2 Hz) |
|---|---|
| Marker_2 | Coh. O1-F4 (30.6-31.6 Hz)/Coh. O2-F3 (29.4-30.4 Hz) |
| Marker_3 | Coh. O2-C3 (19.8-20.8 Hz)/Coh. O2-F3 (11.8-12.8 Hz) |
| Marker_4 | Coh. C3-F4 (37.8-38.8 Hz)/Coh. C4-F3 (39.6-40.6 Hz) |
| Intercept | −89.8 |
| Slope for Marker_1 | 11.45 |
| Slope for Marker_2 | 63.1 |
| Slope for Marker_3 | 5.89 |
| Slope for Marker_4 | 57.14 |

FIG. 20A-D shows graphically the 4 markers that are combined to produce symptom severity PTSD_Symptom_S2 computed during Stage 2 sleep and using 1 Hz bandwidth in the coherence analysis. A scatter plot and regression line of PTSD_Symptom_S2, computed from the markers of FIG. 20A-D vs. PCL-5, are shown in FIG. 20E. The relationship between the Neuromarker and PCL-5 was significant with R² of 0.53 (F=96 p<10⁻⁶).

### Combining Awake and Stage 2 Using Single Frequencies

A new PTSD diagnostic neuromarker was developed based on the product of S2 and Awake Neuromarkers:

### PTSD_Diag_S2×PTSD_Diag_Awake

Table 16 below shows the statistical comparison of PTSD_Diag: S2×W between the two groups:

TABLE 16

| PTSD Diag: S2 × W | Control (n = 38) | PTSD (n = 38) |
|---|---|---|
| Mean (Std) | 1.13 (0.26) | 3.75 (0.45) |
| Median | 1.16 | 3.69 |

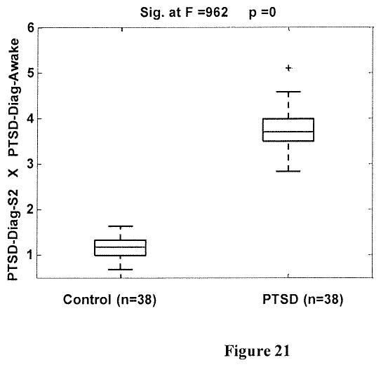

ANOVA analysis between the PTSD_Diag_S2×PTSD_Diag_Awake of the two groups is shown in FIG. 21, a box plot comparison of a combined awake and sleep PTSD diagnostic Neuromarker, computed from the product of PTSD_Diag_S2 and PTSD_Dia_Awake in each individual and based on single frequency coherence analysis, between the control and PTSD groups. On each box, the central mark is the median, the edges of the box are the 25th and 75th percentiles, the whiskers extend to the most extreme data-points the algorithm considers to be not outliers, and the outliers are plotted individually. ANOVA showed that the mean of the neuromarker of PTSD was significantly larger than the control group (sig. at F=962, p<10\hat{ }⁻⁸). Based on the ANOVA analysis, values of 2.3 and above appear to correspond to the presence of PTSD.

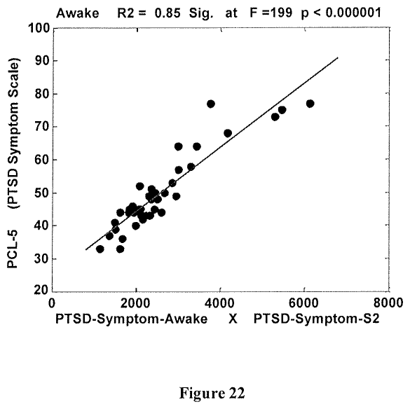

A new PTSD Symptom neuromarker was developed based on the product of S2 and Awake Neuromarkers:
PTSD_Symptom_S2×PTSD_Symptom_Awake.
   FIG. 22 shows the scatter plot and regression line of the combined awake and sleep neuromarker, PTSD_Symptom_S2×PTSD_Symptom_Awake (single frequency analysis) vs. PCL-5. The relationship between the Neuromarker and PCL-5 was strong and significant with R² of 0.85 (F=199 p<10⁻⁶).

### Combining Awake and Stage 2 Using 1 Hz Frequency Band

A new marker was developed based on the product of S2 and Awake Neuromarkers:

### PTSD_Diag_S2×PTSD_Diag_Awake

Table 17 below shows the statistical comparison of PTSD_Diag: S2×W between the two groups:

| PTSD_Diag: S2 × W | Control (n = 38) | PTSD (n = 38) |
|---|---|---|
| Mean (Std) | 1.12(0.22) | 3.76 (0.48) |
| Median | 1.12 | 3.56 |

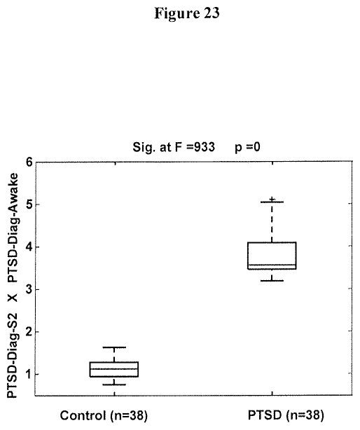

ANOVA analysis between the PTSD_Diag_S2×PTSD_Diag_Awake of the two groups is shown in FIG. 23. FIG. 23 is a box plot comparison of a combined awake and sleep PTSD diagnostic Neuromarker, computed from the product of PTSD_Diag_S2 and PTSD_Diag_Awake in each individual and based on 1-Hz frequency band-width coherence analysis, between the control and PTSD groups. On each box, the central mark is the median, the edges of the box are the 25th and 75th percentiles, the whiskers extend to the most extreme data-points the algorithm considers to be not outliers, and the outliers are plotted individually. ANOVA showed that the mean of the neuromarker of PTSD was significantly larger than the control group (sig. at F=933, p<10\hat{ }⁻⁸). Based on the ANOVA analysis, values of 2.5 and above appear to correspond to the presence of PTSD.

A new PTSD Symptom neuromarker was developed based on the product of S2 and Awake Neuromarkers.

### PTSD_Symptom S2×PTSD_Symptom Awake.

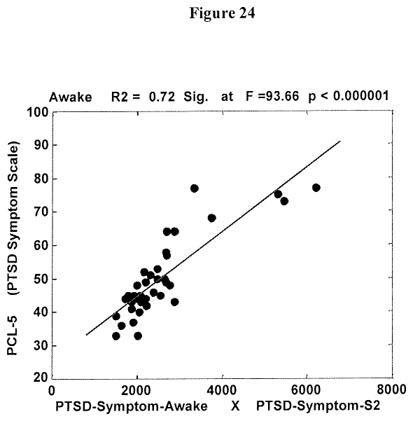

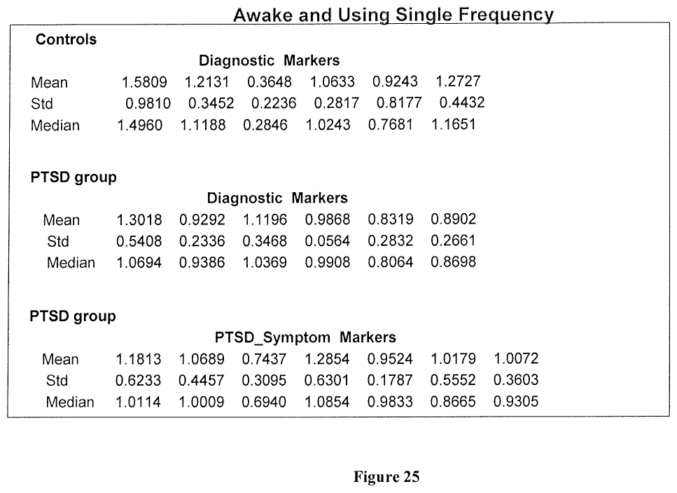

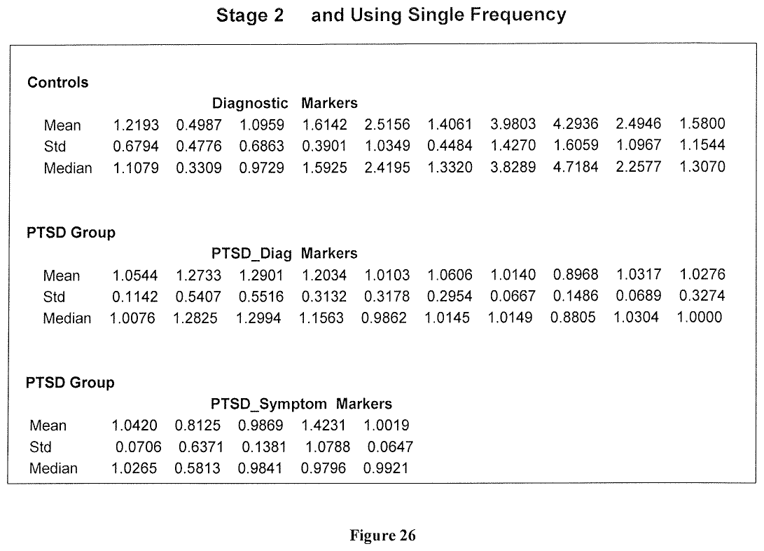

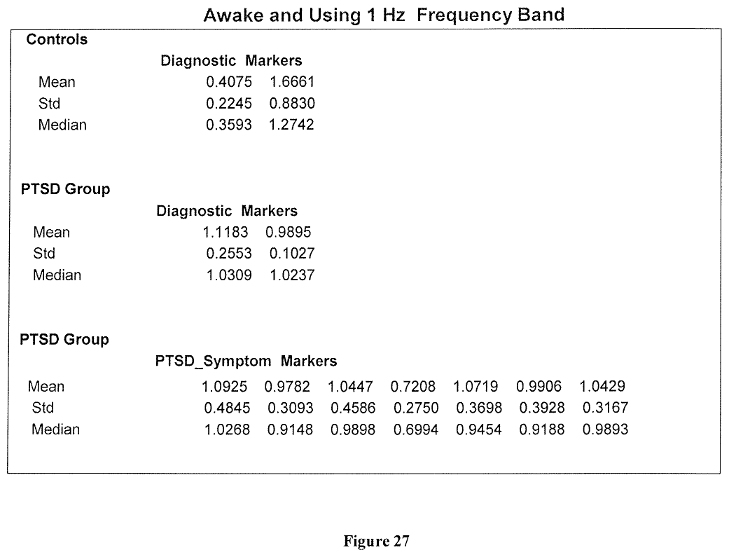

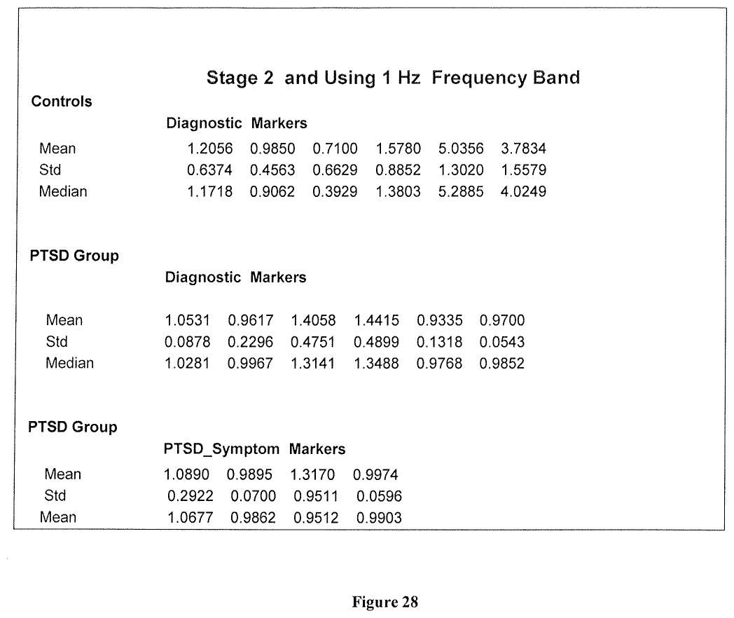

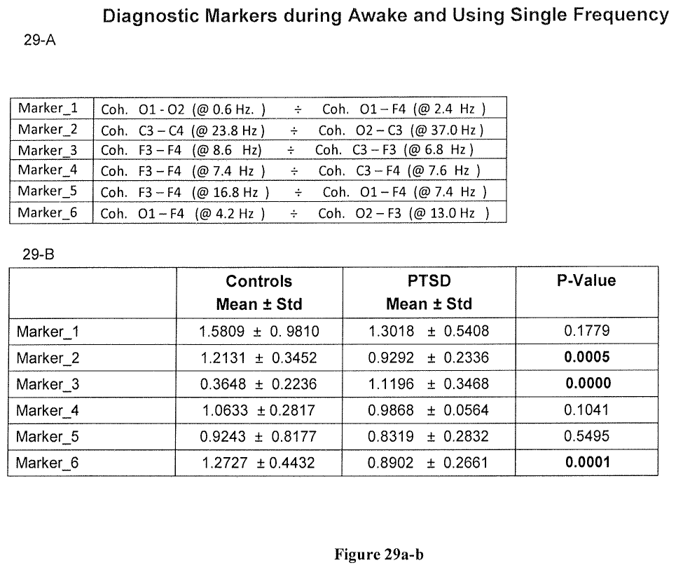

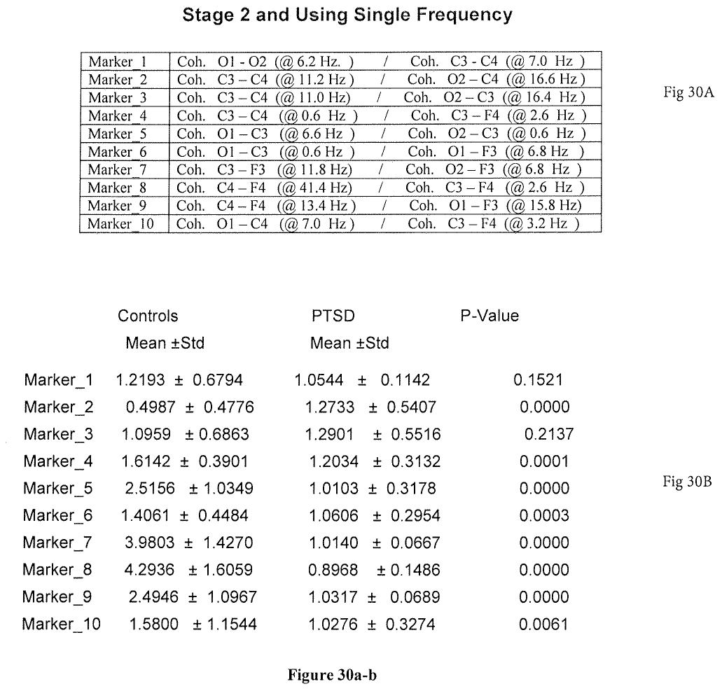

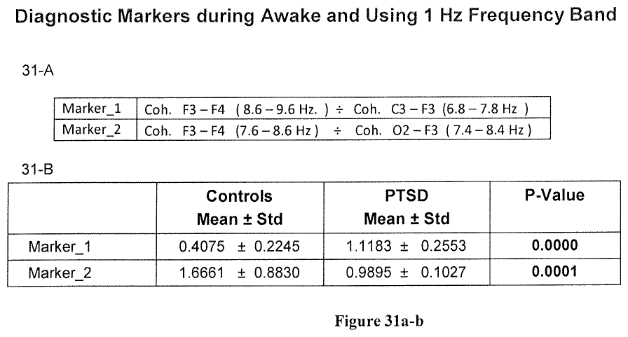

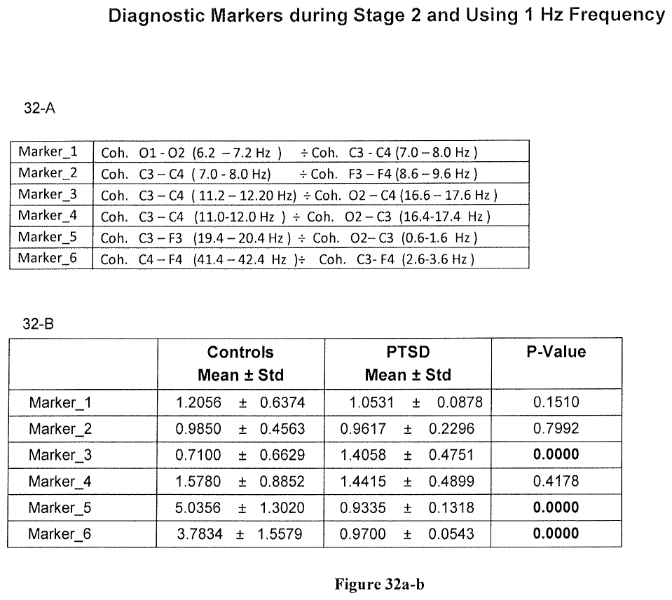

FIG. 24 shows the scatter plot and regression line of the combined awake and sleep neuromarker, PTSD_Symptom_S2×PTSD_Symptom_Awake (1 Hz band analysis) vs. PCL-5. The relationship between the Neuromarker and PCL-5 was strong and significant with R² of 0.72 (F=94 p<10⁻⁷).

### Example 5

Neuromarkers Associated with Diagnosis of PTSD Using REM Sleep
We define “PTSD_Diag_REM” as one Neuromarker that distinguished PTSD from control from data obtained during REM sleep. This particular Neuromarker is computed from a combination of the below 5 markers using multiple linear regression, shown in the Table 18 below. Table 18 bottom contains the intercept and slope coefficients for each of the 5 markers used in the multiple linear regression.

TABLE 18

| | |
|---|---|
| Marker_1 | Coh. C3-F3 (@ 17.8 Hz.)/Coh. C4-F4 (@ |
| | 18.4 Hz) |
| Marker_2 | Coh. C3-F3 (@ 19.4 Hz)/Coh. O2-C3 (@ |
| | 2.6 Hz) |
| Marker_3 | Coh. C3-F3 (@ 22.2 Hz)/Coh. O1-F3 (@ |
| | 15.2 Hz) |
| Marker_4 | Coh. C4- F4 (@ 8.0 Hz)/Coh. O1-F4 (@ 9.2 |
| | Hz) |
| Marker_5 | Coh. O1-C4 (@ 6.8 Hz)/Coh. O2-C3 (@ 2.6 |
| | Hz) |
| Intercept | 2.1 |
| Slope for | 0.26 |
| Marker _1 | |
| Slope for | −0.09 |
| Marker_2 | |
| Slope for | −0.08 |
| Marker_3 | |
| Slope for | −0.12 |
| Marker_4 | |
| Slope for | −0.15 |
| Marker_5 | |

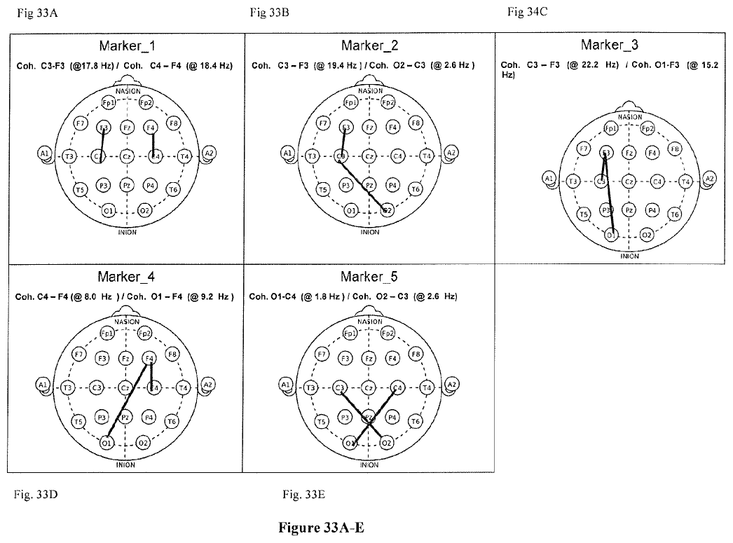

FIG. 33 shows graphically the 5 markers that are included in the PTSD_Diag_REM Neuromarker.
Table 19 below shows the statistical comparison of PTSD_Diag_REM between the two groups:

| | | | Regression Statistics: | | |
|---|---|---|---|---|---|
| | Control | PTSD | | F | p |
| PTSD_Diag_REM | (n = 31) | (n = 29) | R² | value | value |
| Mean (Std) | 1.05 (021) | 1.94 (0.0.07) | 0.89 | 86.6 | 0.0000 |
| Median | 1.08 | 1.94 | | | |

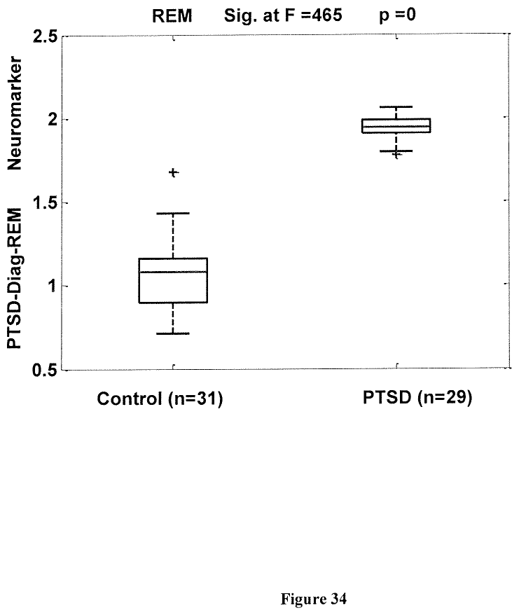

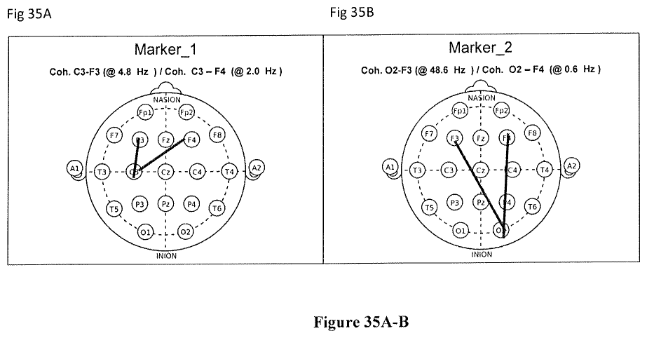

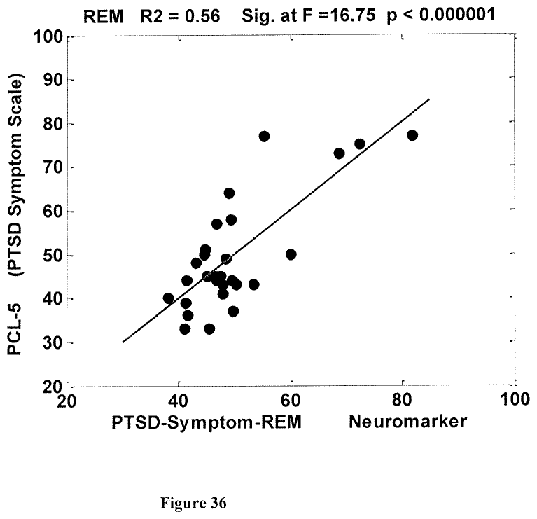

ANOVA analysis between the PTSD_Diag_REM shown below. In this figure PTSD_Diag_REM values of the two groups are shown in boxplot format, the top and bottom lines of boxes representing the 75^{th} and 25^{th} percentiles, median value shown as a red line, and the whiskers depict the range. Based on the regression and ANOVA analysis, values of 1.75 and above for PTSD_Diag_REM Neuromarker appear to correspond to the presence of PTSD (FIG. 34).
Neuromarkers Associated with Symptom Severity of PTSD Using REM Sleep
This particular Neuromarker is computed from a combination of the 2 markers using multiple linear regression, shown in Table 20 and FIGS. 35 and 36. Table 20, bottom, contains the intercept and slope coefficients for each of the 2 markers used in the multiple linear regression. For these analysis the dependent variable was PCL-5 (patient symptoms), and the independent variable was PTSD-Symptom-S2 based on combination of the most sensitive markers to PTSD.

TABLE 20

| | |
|---|---|
| Marker_1 | Coh. C3-F3 (@ 4.8 Hz)/Coh. C3-F4 (@ 2.0 Hz) |
| Marker_2 | Coh. O2-F3 (@ 48.6.8 Hz)/Coh. O2-F4 (@ |
| | 0.6 Hz) |
| Intercept | 8.6 |
| Slope for | 26.73 |
| Marker_1 | |
| Slope for | 9.77 |
| Marker_2 | |

### Application of Neuromarkers to Clinical Sleep Laboratory or Doctor's Office

The Neuromarkers of this method appear to be sensitive to the awake state with eyes closed before falling asleep, as well as during sleep (in particular stage 2). The results show that the Neuromarkers obtained during sleep are highly sensitive to the presence of PTSD, such as PTSD_Diag_S2 of FIG. 15K. With a threshold of 1.75 where the Neuromarkers above this level point at the presence of PTSD. With regards to PTSD severity quantification, the awake period before sleep seems to be a better period for severity quantification, such as FIG. 10 where PTSD_Symptom_Wake was very strongly correlated with PTSD symptoms (according to PCL-5) with an R² of 0.85 (corresponding to a correlation coefficient of 92%).

For the purpose of determination of the presence of PTSD, the method can be used at a clinical sleep laboratory, anywhere in the clinic or hospital unit with a bed, or at the patient's own home. For assessing the severity of the symptoms, as well as tracking the effect of therapy and recovery, the method can be applied in the same setting as above, but also anywhere that has a comfortable couch/bed so that the patient can close their eyes and take a 15-30 minute nap, such as within the setting of a doctor's office visit.

### REFERENCES

1. DSM, American Psychiatric Association: Diagnostic and Statistical Manual of Mental Disorders, 4th Edition, Text Revision (2000). Washington, D.C., American Psychiatric Publishing.
2. Kessler et al., (1995). Posttraumatic stress disorder in the National Comorbidity Survey. Arch Gen Psychiatry, 52(12): 1048-1060.
3. Resnick et al., (1993). Prevalence of civilian trauma and post-traumatic stress disorder in a representative national sample of women. J Consult Clin Psycho!, 61(6):984-991.
4. Dohrenwend et al., (2006). The psychological risks of Vietnam for U.S. veterans: A revisit with new data and methods. Science, 313(5789):979-982.
5. Milliken et al., (2007). Longitudinal assessment of mental health problems among active and reserve component soldiers returning from the Iraq war. J Am Med Assoc, 298(18):2141-2148.
6. Foa et al., (2007). Prolonged exposure therapy for PTSD: Emotional processing of traumatic experiences therapist guide. New York, N.Y.: Oxford University Press.
7. Powers et al., (2010). A meta-analytic review of prolonged exposure for posttraumatic stress disorder. Clinical Psychology Review, 30(6), 635-641.
8. Ballinger et al., (2004). Consensus statement update on posttraumatic stress disorder from the international consensus group on depression and anxiety. J Clinical Psychiatry, 65, 55-62.
9. Nemeroff et al., (2006). Posttraumatic stress disorder: A state-of-the-science review. Journal of Psychiatric Research, 40, 1-21.
10. Foa et al., (2009). Effective treatments for PTSD: Practice guidelines from the International Society for Traumatic Stress Studies (2nd ed.). New York: Guilford Press.
11. NICE. (2005). Post-traumatic stress disorder (PTSD): The management of PTSD in adults and children in primary and secondary care. London: National Institute for Clinical Excellence.
12. Treatment for Posttraumatic Stress Disorder in Military and Veteran Populations (2012), Institute of Medicine, National Academy of Science: ISBN 978-0-309-25421-2.
13. Ohayon et al., (2000). Sleep disturbances and psychiatric disorders associated with posttraumatic stress disorder in the general population. Comprehens Psychiatry, 41: 469-78.
14. Kilpatrick et al., (1998). Posttraumatic stress disorder field trial: evaluation of PTSD construct criteria A through E. In: Widiger T A, Frances A J, Pincus H A, Ross R, First M B, Davis W, et al., editors. DSM-IV sourcebook. Washington: American Psychiatric Press.
15. Harvey et al., (2003): Sleep and posttraumatic stress disorder: a review. Clin Psycho!Rev, 23:377-407.
16. American Academy of Sleep Medicine. (2005). The international classification of sleep disorders: Diagnostic and coding manual. 2nd ed. Westchester (IL): American Academy of Sleep Medicine.
17. Germaine et al., (2004): Clinical correlates of poor sleep quality in posttraumatic stress disorder. J Trauma Stress, 17:477-484.
18. Koren et al., (2002): Sleep complaints as early predictors of posttraumatic stress disorder: a I-year prospective study of injured survivors of motor vehicle accidents. Am J Psychiatry, 159:855-857.
19. Mellman et al., (2002): REM sleep and the early development of posttraumatic stress disorder. Am J Psychiatry, 159:1696-1701.
20. Mellman et al., (2004): Heart rate variability during sleep and the early development of posttraumatic stress disorder. Biol Psychiatry, 55:953-956.
21. Spoormaker et al., (2008). Disturbed Sleep in Post-Traumatic Stress Disorder: Secondary Symptom or Core Feature. Sleep Medicine Reviews, 12, 169-184.
22. Lydiard et al., (2009). Clinical Importance of Sleep Disturbance as a treatment target in PTSD. J Lifelong Learning in Psychiatry, 7(2): 176-183.
23. Zayfert et al., (2004). Residual insomnia following cognitive behavioral therapy for PTSD. J Traumat Stress, 17: 69-73.
24. Jacobs-Rebhun et al., (2000). Posttraumatic stress disorder and sleep difficulty. Am J Psychiatry, 157:1525-6.
25. Kobayashi et al., (2007). Polysomnographically measured sleep abnormalities in PTSD: A meta-analytic review. Psychophysiology, 44:660-9.
26. Breslau et al., (2004). Sleep in lifetime posttraumatic stress disorder: a community-based polysomnographic study. Arch Gen Psychiatry, 61:508-16.
27. Mellman et al., (1995). Sleep events among veterans with combat related posttraumatic stress disorder. Am J Psychiatry, 152:100-5.
28. Anne Germaine (2013). Sleep Disturbance as the Hallmark of PTSD: Where Are We know. Am J Psychiatry, 170: 372-382.
29. Othmer et al., (2011). Post Traumatic Stress Disorder—The Neurofeedback Remedy. Biofeedback, 37(1):24-31.
30. Bendat et al., (1980). Engineering Applications of Correlation and Spectral Analysis. Wiley, New York.
31. Nunez et al., (1997). EEG coherency. I. Statistics, reference electrode, volume conduction, Laplacians, cortical imaging, and interpretation at multiple e scales. Electroencephalogr Clin Neurophysiol, 103:499-515.
32. Thatcher et al., (1988) Biophysical Linkage between MRI and EEG Coherence in Closed Head Injury. Neuroimage 8, 307-326.
33. Suzuki (1974). Phase relationships of alpha rhythm in man. Jpn J Physiol, 24(6):569-586.
34. U.S. Pat. No. 6,993,380.
35. U.S. Pat. No. 7,860,561.
36. U.S. Pat. No. 8,335,561.
37. Modarres et al., (2004). “Novel Computerized EEG Algorithms for High Resolution Characterization of Wake-Sleep Transitions”, Sleep, 27(A369), Abstract Supplement, 2004.
38. Modarres (2005). A Neuro-behavioral Test and Algorithms for Quantification of Sleepiness and Characterization of Wake-Sleep Transitions. Proceedings of the 2005 IEEE Engineering in Medicine and Biology 27th Annual Conference. Shanghai, China, Sep. 1-4, 2005.
39. Agrawal et al., (2011), “NREM Sleep Staging Using WAVCNS Index.” J Clinical Monitoring and Computing, 25(2): 137-142.
40. Rechtschaffen A, Kales A, editors. (1968) Los Angeles: Brain Information Service/Brain Research Institute, University of California. A manual of standardized terminology, techniques and scoring system of sleep stages in human subjects.
41. Iber C, Ancoli-Israel S, Chesson A, Quan S F, editors. (2007) 1st ed. Westchester, Ill.: American Academy of Sleep Medicine. The AASM manual for the scoring of sleep and associated events: rules, terminology, and technical specification
42. Weathers et al., (1993). The PTSD Checklist (PCL): Reliability, validity, and diagnostic utility. Paper presented at the Ninth Annual Meeting of the International Society for Traumatic Stress Studies, San Antonio, Tex.
43. National Center for PTSD: Using the PTSD Checklist: http://www.ptsd.va.gov/professional/pages/assessments/assessment-pdf/pcl-handout. Pdf
44. Bliese, et al., (2008). Validating the primary care posttraumatic stress disorder screen and the posttraumatic stress disorder checklist with soldiers returning from combat. J Consulting and Clinical Psychology, 76, 272-281.
45. Walker et al., (2002). Validation of the PTSD checklist in an HMO sample of women. General Hospital Psychiatry, 24, 375-380.
46. Freedy et al., (2010). Post-traumatic stress disorder screening test performance in civilian primary care. Family Practice.
47. Weathers, Frank W., et. al (2014). PTSD Checklist for DSM-5 (PCL-5). VA National Center for PTSD Website
48. DSM-5 (2013): Diagnostic and Statistical Manual of Mental Disorders (Fifth ed.). American Psychiatric Association. Arlington, Va.: American Psychiatric Publishing.

What is claimed is:

1. A method for detecting post-traumatic stress disorder (PTSD) in a subject comprising the steps of:
a) obtaining two or more brain wave patterns from at least two locations selected on a head of a subject;
b) segmenting the brain wave patterns by sleep stage;
c) segmenting the brain wave patterns for one sleep stage so segmented from step (b) into defined time intervals, so as to permit auto and cross spectral analysis and/or coherence analysis;
d) calculating coherence value(s) and/or phase delay value(s) from two brain wave segments of step (c) for a single frequency or a frequency band; and
e) determining if the coherence value(s), phase delay value(s) and/or a combination thereof is above or below a designated threshold, so as to determine presence of PTSD in the subject;
thereby, detecting post-traumatic stress disorder in the subject.

2. (canceled)

3. The method of claim 1, wherein the brain wave patterns are obtained from analysis of the subject's brain function during sleep, awake-to-sleep and/or awake to sleep initiation.

4. The method of claim 3, wherein the brain function is assessed with the use of polysomnography which comprises electroencephalography (EEG).

5. The method of claim 4, wherein electroencephalography comprises placement of at least two EEG electrodes on at least two head or scalp locations.

6. The method of claim 5, wherein the head or scalp locations are selected from head or scalp electrode placement locations according to International 10-20 system.

7. The method of claim 6, wherein head or scalp electrode placement according to International 10-20 system consists of head or scalp locations, Fp1, Fp2, F3, F4, F7, F8, Fz, A1, A2, C3, C4, Cz, T3, T4, T5, T6, P3, P4, Pz, O1 and O2.

8. The method of claim 1, wherein head or scalp locations are selected from head or scalp locations F3, F4, C3, C4, O1 and O2.

9. The method of claim 1, wherein brain wave patterns are obtained from six scalp locations using EEG electrodes placed at scalp locations F3, F4, C3, C4, O1 and O2.

10. The method of claim 1, wherein the brain wave patterns are recorded simultaneously.

11. The method of claim 1, wherein the brain wave patterns in step (b) are segmented into sleep stage selected from the group consisting of awake period with lights off and before falling asleep (W), stage I sleep, stage II sleep (S2), delta-wave or stable III sleep and rapid-eye-movement (REM) sleep.

12. The method of claim 1, wherein the sleep stage in step (c) is selected from the group consisting of awake period with lights off and before falling asleep (W), stage I sleep, stage II sleep (S2), delta-wave or stable III sleep and rapid-eye-movement (REM) sleep.

13.-15. (canceled)

16. The method of claim 1, wherein the defined time interval in step (c) is greater than 2 seconds and less than 30 seconds.

17. (canceled)

18. The method of claim 1, wherein the single frequency is selected from any frequency between 0 Hz and 52 Hz.

19.-56. (canceled)

57. The method of claim 1, wherein the designated threshold is determined from comparison of set of values from control subjects and a second set of values from PTSD subjects and its value is set such as to permit discrimination between control and PTSD subjects.

58.-65. (canceled)

66. The method of claim 1, wherein the subject with coherence value(s), phase delay value(s) and/or a combination thereof above a designated threshold is considered to have PTSD for mean, median and/or mode of coherence values, phase delay values and/or a combination thereof of control subjects below PTSD subjects, or alternatively below a designated threshold is considered to have PTSD for mean, median and/or mode of coherence values, phase delay values of control subjects above PTSD subjects.

67.-80. (canceled)

81. The method of claim 1, wherein the combination thereof of coherence value(s) and/or phase delay values of step (e) is multiple linear regression of coherence value(s) and/or phase delay values or multiple linear regression of markers, respectively.

82.-126. (canceled)

127. The method of claim 1, wherein the designated threshold is a value or absolute value and wherein the value of the neuromarker is a value or absolute value.

128.-343. (canceled)

344. A method for detecting post-traumatic stress disorder (PTSD) in a subject comprising the steps of:
a. obtaining two or more brain wave patterns from at least two locations selected on a head of a subject;
b. segmenting the brain wave patterns by sleep stage;
c. segmenting the brain wave patterns for one sleep stage so segmented from step (b) into defined time intervals, so as to permit auto and cross spectral analysis and/or coherence analysis;
d. calculating coherence value(s) and/or phase delay value(s) from two brain wave segments of step (c) for a single frequency or a frequency band at a sleep stage; and
e. repeating step (d) to obtain more coherence values and/or phase delay values for the same sleep stage, at (i) other single frequency or frequency band obtained from the same two locations, and/or (ii) the same or other single frequency or frequency band obtained from two different locations or two locations in which one location is shared in common in step (d);
f. optionally, performing steps (d) and (e) for a different sleep stage or multiple sleep stages;
g. combining coherence value(s) or phase delay value(s) so as to be a marker or a combination of markers;
h. selecting a neuromarker from the markers or combination of markers of step (g), wherein the neuromarker is defined as: (i) a single coherence value ratio or phase delay value ratio; (ii) combination of two or more markers of step (g) for a sleep stage; and/or (iii) combination of two or more markers of step (g) from two or more sleep stages; and
i. determining if the value of the neuromarker for diagnosing PTSD is above or below a designated threshold, so as to determine presence of PTSD in the subject;
thereby, detecting post-traumatic stress disorder in the subject.

345. The method of claim 344, wherein the brain wave patterns in step (b) are segmented into sleep stage selected from the group consisting of awake period with lights off and before falling asleep (W), stage I sleep, stage II sleep (S2), delta-wave or stable III sleep and rapid-eye-movement (REM) sleep.

346. A system for detecting post-traumatic stress disorder (PTSD), comprising:
an electroencephalogram (EEG) device for measuring brain wave function of a subject;
a storage device; and
a processor communicatively coupled to the storage device, wherein the processor executes application code instructions that are stored in the storage device and that cause the system to:
a) obtain two or more brain wave patterns from at least two locations selected on a head of a subject;
b) segment the brain wave patterns by sleep stage;
c) segment the brain wave patterns for one sleep stage so segmented from step (b) into defined time intervals, so as to permit auto and cross spectral analysis and/or coherence analysis;
d) calculate coherence value(s) and/or phase delay value(s) from two brain wave segments of step (c) for a single frequency or a frequency band; and
e) determine if the coherence value(s), phase delay value(s) and/or a combination thereof is above or below a designated threshold, so as to determine presence of PTSD in the subject;
thereby, detecting PTSD in the subject.
